(19) United States
(12) Patent Application Publication
(10) Pub. No.: US 2025/0230434 A1
(43) Pub. Date: Jul. 17, 2025

(54) STEM CELL DERIVED LINEAGE BARCODING

(71) Applicant: Cairn Biosciences, Inc., San Francisco, CA (US)

(72) Inventors: M. Susana G. DE ABREU RIBEIRO, San Francisco, CA (US); Catherine I. LACAYO, Fremont, CA (US); Mary J. C. LUDLAM, San Francisco, CA (US); Salome Calado BOTELHO, San Francisco, CA (US)

(21) Appl. No.: 18/891,930

(22) Filed: Sep. 20, 2024

Related U.S. Application Data
(63) Continuation of application No. 17/269,223, filed on Feb. 17, 2021, now abandoned.
(60) Provisional application No. 62/765,016, filed on Aug. 18, 2018.

(51) Int. Cl. C12N 15/10; C12N 15/67

(52) U.S. Cl. CPC C12N 15/1086; C12N 15/67; C12N 2830/20; C12N 2840/20

(57) ABSTRACT

The present invention provides multicistronic reporter vectors, acceptor stem cells for receiving multicistronic reporter vectors, and multireporter cells for use in assays for profiling two or more polypeptides in live cells, wherein the vectors comprise a reporter polypeptide under the control of a lineage specific promoter to act as a barcode for a specific cell type. Methods of making multicistronic reporter vectors, acceptor cells for receiving multicistronic reporter vectors, and multireporter cells are provided. Libraries and kits comprising multicistronic reporter vectors, acceptor cells for receiving multicistronic reporter vectors, and multireporter cells are provided. Methods of profiling/assaying the multireporter cells and multireporter cell libraries are provided.

### CROSS-REFERENCE TO RELATED APPLICATIONS

This application claims the benefit of U.S. Provisional Patent Application No. 62/765,016 filed Aug. 18, 2018, the disclosure of which is hereby incorporated by reference in its entirety.

### STATEMENT REGARDING FEDERALLY SPONSORED RESEARCH OR DEVELOPMENT

This invention was made with government support under grant no. R44TR002572-03 awarded by the National Institutes of Health. The government has certain rights in the invention.

### TECHNICAL FIELD

The present invention relates to methods for making and using stem cell derived multicolor reporter cells lines containing a lineage barcode to discriminate distinct cell lineages in live co-cultures to monitor pathways, phenotypes, assays, compound mode of action and model disease; for example, in live cells in real time.

### BACKGROUND

Ascertaining cellular specific responses to internal or external stimuli necessitates visualization and monitoring of cell types, phenotypes and pathways and the dynamic interplay between them. Current methods of visualizing such responses are inefficient, costly and provide very limited insight into multiparametric, dynamic processes occurring at the cellular and molecular level. For example, drug discovery and/or toxicological evaluation of drug candidates is traditionally carried out using in vivo preclinical animal models that are low-throughput, costly, inadequately predictive of toxicity in humans and offer little insight into compound therapeutic mechanism of action or compound toxicity liabilities. Similarly routinely used cell-based assays such as Western blots provide endpoint readouts of processes that would be better characterized an understood using temporal profiling in live cells and are also extremely limited in throughput. The throughput of these traditional approaches is far outpaced by the rate at which new chemicals are generated, be they compounds developed for pharmaceutical, agricultural or other purposes such as nutrition, cosmetics, personal care, etc. The need for these new chemicals to be evaluated for efficacy or for the risks they may pose to human health mandates the development of new screening tools that can offer meaningful insights into the mechanisms associated with such compounds at a throughput compatible of evaluating hundreds of thousands of compounds per week. Although cell-based approaches such as immunofluoresence microscopy and viability assays have been demonstrated in high-throughput formats, these approaches are still limited to endpoint readouts that are assayed in dead cell systems, severely limiting their physiological relevance and greatly constraining their suitability for profiling complex multiparametric processes in disease relevant living cells. Ideally new screening tools would be capable of addressing the critic lack of tools to enable visualization and quantification of changes in specific cellular physiology in physiologically relevant cell-based models such as those enabled by stem cell-based as disease models. These needs can be addressed by an appropriately configured stem cell live-cell approach, which is the focus of the present invention.

The invention summarized provides the means to implement a lineage-specific tagging approach, termed lineage barcoding, to robustly discriminate distinct cell lineages in live stem cell cultures and co-cultures to establish an assay for compound profiling and drug target discovery, as a model for drug-discovery and toxicology screens requiring high-throughput technologies.

Multiplex assays are disclosed in PCT/US2018/032834, incorporated by reference herein in its entirety.

All references cited herein, including patent applications and publications, are incorporated by reference in their entirety.

### SUMMARY

In some aspects, the invention provides a multicistronic reporter vector comprising: a promoter operably linked to an open reading frame, wherein the promoter is a lineage-specific promoter, wherein the open reading frame comprises two or more cistrons, and wherein expression of the open reading frame in a cell yields separate component polypeptide products from each cistron; wherein each cistron comprises a multiple cloning site (MCS) and nucleic acid encoding a reporter vector, wherein each cistron encodes a different reporter polypeptide; and wherein expression of two or more nucleic acids encoding polypeptides inserted into the two or more multiple cloning sites and fused to the reporter polypeptides is essentially at about 1:1 stoichiometry.

In some aspects, the invention provides a multicistronic reporter vector comprising: first promoter linked to a transactivator polypeptide, wherein the first promoter is a lineage-specific promoter; a second promoter operably linked to an open reading frame, wherein the second promoter is inducible e transactivator polypeptide, wherein the open reading frame comprises two or more cistrons, and wherein expression of the open reading frame in a cell yields separate component polypeptide products from each cistron: wherein each cistron comprises a multiple cloning site (MCS) and nucleic acid encoding a reporter vector, wherein each cistron encodes a different reporter polypeptide; and wherein expression of two or more nucleic acids encoding polypeptides inserted into the two or more multiple cloning sites and fused to the reporter polypeptides is essentially at about 1:1 stoichiometry. In some embodiments, the transactivator polypeptide is a tetracycline transactivator polypeptide and the second promoter comprises a tetracycline responsive element. In some embodiments, the tetracycline responsive element is a Tet operator 2 (TetO2) inducible or repressor element.

In some aspects, the invention provides a multicistronic reporter vector comprising: a first promoter linked to a nucleic acid encoding an organelle-specific polypeptide, wherein the first promoter is a lineage-specific promoter; a second promoter operably linked to an open reading frame, wherein the second promoter is a constitutive promoter, wherein the open reading frame comprises two or more cistrons, and wherein expression of the open reading frame in a cell yields separate component polypeptide products from each cistron, wherein each cistron comprises a multiple cloning site (MCS) and nucleic acid encoding a reporter vector, wherein each cistron encodes a di reporter polypeptide; and wherein expression of two or more nucleic acids encoding polypeptides inserted into the two or more multiple cloning sites and fused to the reporter polypeptides is essentially at about 1:1 stoichiometry. In some embodiments, the organelle-specific polypeptide is H2B. In some embodiments, the constitutive promoter is a Cytomegalovirus a (CMV), a Thymidine Kinase (TK), an eF1-alpha, a Ubiquitin C (UbC), a Phosphoglycerate to Kinase (PGK), a CAG promoter, an SV40 promotor, or a human β-actin promoter. In some embodiments, the promoter comprises a tetracycline responsive element. In some embodiments, the tetracycline responsive element is a Tet operator 2 (TetO2) inducible or repressor element.

In some embodiments of the above aspects and embodiments, the first promoter and the second promoter are in different orientations. In some embodiments, the first promoter and the second promoter are separated by an insulator nucleic acid. In some embodiments, the cistrons are separated from one another by nucleic acid encoding one or more self-cleaving peptide and/or one or more internal ribo entry site (IRES). In some embodiments, the one or more self-cleaving tides is a viral self-cleaving peptide. In some embodiments, the one or more viral self-cleaving peptides is one or more 2A peptides. In some embodiments, one or more 2A peptides is a T2A peptide, an E2A peptide or a F2A peptide.

In some embodiments, the reporter polypeptide further comprises one or more nucleic acids encoding a peptide linker between one or more of the reporter polypeptides and one or more of the self-cleaving peptides. In some embodiments, the peptide linker comprises the sequence Gly-Ser-Gly.

In some embodiments, the reporter polypeptide is a fluorescent reporter polypeptide. In some embodiments, the reporter polypeptide for each cistron is selected from GFP, EGFP, Emerald, Citrine, Venus, mOrange, mCherry, TagBFP, mTurquoise, Cerulean, UnaG, dsRed, eqFP611, Dronpa, RFP, TagRFPs, TdTomato, KFP, EosFP, Dendra, IrisFP, iRFPs and smURFP.

In some embodiments, the open reading frame comprises a first cistron and a second cistron, where h cistron comprises 5′ to 3′ nucleic acid comprising a MCS, nucleic acid encoding a reporter polypeptide, and nucleic acid encoding a linker peptide; wherein the first cistron and the second cistron are separated by nucleic acid encoding a viral cleavage peptide. In some embodiments, the open reading frame comprises a first cistron, a second cistron and a third cistron, wherein each cistron comprises 5′ to 3′ nucleic acid comprising a MCS, nucleic acid encoding a reporter polypeptide, and nucleic acid encoding a linker peptide; wherein the first cistron and the second cistron are separated by nucleic acid encoding a first viral cleavage peptide and the second cistron and the third cistron are separated by nucleic acid encoding a second viral cleavage peptide. In some embodiments, the open reading frame comprises a first cistron, a second cistron, a third cistron and a fourth cistron, wherein each cistron comprises 5′ to 3′ nucleic acid comprising a MCS, nucleic acid encoding a reporter polypeptide nucleic acid encoding a linker peptide; wherein the first cistron and the second cistron are separated by nucleic acid encoding a first viral peptide, the second cistron and the third cistron are separated by nucleic acid encoding a second viral cleavage peptide the third cistron and the fourth cistron are separated by nucleic acid encoding a third viral cleavage peptide. In some embodiments, the vector comprises a promoter operably linked to an open reading frame, wherein the open reading frame comprises a first cistron, a second cistron, a third cistron and a fourth cistron, wherein each cistron comprises 5′ to 3′ nucleic acid comprising a MCS, nucleic acid encoding a reporter polypeptide, nucleic acid encoding a linker peptide; wherein the first cistron and the second cistron are separated by nucleic acid encoding a first viral cleavage peptide, the second cistron and the third cistron are separated by nucleic acid encoding a second viral cleavage peptide the third cistron and the fourth cistron are separated by nucleic acid encoding an IRES.

In some embodiments, the lineage-specific promoter is specific for cells of heart, blood, muscle, lung, liver, kidney, pancreas, brain, or skin lineage. In some embodiments, the lineage specific promoter is a sublineage-specific promoter. In some embodiments, lineage-specific promoter is a cardiac specific promoter. In some embodiments, the cardiac-specific promoter is a MCLV2v, a SLN, a SHOX2, a MYBPC3, a TNNI3 or an α-MHC promoter. In some embodiments, the lineage-specific promoter is a neural specific promoter. In some embodiments, neural-specific promoter is a vGAT, a TH, a GFAP, or a vGLUT1 promotor.

In some embodiments, the vector further comprises a site-specific recombinase sequence located 3′ to the open reading frame. In some embodiments, the vector further comprises nucleic acid encoding a selectable marker, wherein the nucleic acid encoding the selectable marker is not operably linked to the promotor when the site-specific recombinase sequence has not recombined and is operably linked to the promoter when the site-specific recombinase sequence recombines with its target-site specific recombinase sequence. In some embodiments, the site-specific recombinase sequence is a FRT nucleic acid sequence and/or an attP nucleic acid and/or a loxP nucleic acid sequence. In some embodiments, the selectable marker confers resistance to hygromyocin, Zeocin™, puromycin, neomycin or an analog of hygromyocin, Zeocin™, puromycin, blasticidin or neomycin. In some embodiments, nucleic acid encoding one or more polypeptides is inserted in-frame into the one or more MCS. In some embodiments, at least one cistron comprises nucleic acid encoding a housekeeping gene. In some embodiments, the housekeeping gene is H2B. In some embodiments, at least one cistron comprises nucleic acid encoding an organelle marker. In some embodiments, the organelle marker comprises H2B, α-actinin 2 or a mitochondrial targeting signal fused to the reporter polypeptide.

In some embodiments, the one or more polypeptides comprise polypeptides that can be used to profile or distinguish a single or multiple biological pathways, cross-talk between two or more biological pathways, synthetic lethality, cellular homeostasis, organelle homeostasis, other cellular or subcellular phenotypes, cell-cell interactions or a toxicity response.

In some aspects, the invention provides a multireporter stem cell, wherein the multireporter stem cell comprises a multicistronic reporter construct, the multicistronic reporter construct a promoter operably linked to an open reading frame, wherein the promoter is a lineage-specific promoter, wherein the open reading frame comprises two or more cistrons, and wherein expression of the open reading frame in a cell yields separate component polypeptide products from each cistron; wherein n comprises a multiple cloning site (MCS) and nucleic acid encoding a reporter vector, wherein h cistron encodes a different reporter polypeptide; wherein expression of two or more nucleic acids encoding polypeptides inserted into the two or more multiple cloning sites and fused to the reporter polypeptides is essentially at about 1:1 stoichiometry; and wherein the stem cell is a pluripotent stem cell, a multipotent stem cell or an induced pluripotent stem (IPS) cell.

In some aspects, the invention provides a multireporter stem cell, wherein the multireporter stem cell comprises a multicistronic reporter construct, wherein the multicistronic reporter construct comprises a first promoter linked to a transactivator polypeptide, wherein the first promoter is a lineage-specific promoter; a second promoter operably linked to an open reading frame, wherein second promoter is inducible by the transactivator polypeptide, wherein the open reading frame comprises two or more cistrons, and wherein expression of the open reading frame in a cell yields separate component polypeptide products from each cistron; wherein each cistron comprises a multiple cloning site (MCS) and nucleic acid encoding a reporter vector, wherein each cistron encodes a different reporter polypeptide; wherein expression of two or more nucleic acids encoding polypeptides inserted the two or more multiple cloning sites and fused to e reporter polypeptides is essentially at about 1:1 stoichiometry; and wherein the stem cell is a pluripotent stem cell, a multipotent stem cell or an induced pluripotent stem (iPS) cell. In some embodiments, the transactivator polypeptide is a tetracycline transactivator polypeptide and the second promoter comprises a tetracycline responsive element. In some embodiments, the tetracycline responsive element is a Tet operator 2 (TetO2) inducible or repressor element.

In some aspects, the invention provides a multireporter stem cell, wherein the multireporter stem cell comprises a multicistronic reporter construct, wherein the multicistronic reporter construct comprises a first promoter linked to a nucleic acid encoding a housekeeping polypeptide, wherein the first promoter is a line-specific promoter; second promoter operably linked to an open reading frame, wherein the second promoter is a constitutive promoter, wherein the open reading frame comprises two or more cistrons, and wherein expression of pen reading frame in a cell yields separate component polypeptide products from each cistron; wherein each cistron comprises a multiple cloning site (MCS) and nucleic acid encoding a reporter vector, wherein each cistron encodes a different reporter polypeptide; wherein expression of two or more nucleic acids encoding polypeptides inserted into the two or more multiple cloning sites and fused to the reporter polypeptides is essentially at about 1:1 stoichiometry; and wherein the stem cell is a pluripotent stem cell, a multipotent stem cell or an induced pluripotent stem (IPS) cell. In some embodiments, the housekeeping polypeptide is H2B.

In some embodiments, the constitutive promoter is a Cytomegalovirus a (CMV), a Thymidine Kinase (TK), an of eF1-alpha, a Ubiquitin C (UbC), a Phosphoglycerate Kinase (PGK), a CAG promoter, an SV40 promoter, or a human β-actin promoter. In some embodiments, the first promoter and the second promoter are in different orientations. In some embodiments, the first promoter and the second promoter are separated by an insulator nucleic acid.

In some embodiments, the promoter comprises a tetracycline responsive element. In some embodiments, the tetracycline responsive element is a Tet operator 2 (TetO2) inducible or repressor element.

In some embodiments, the cistrons are separated from one another by nucleic acid encoding one or more self-cleaving peptide and/or one or more internal ribosome entry site (IRES). In some embodiments, self-cleaving peptides is a viral self-cleaving peptide. In some embodiments, the one or more viral self-cleaving peptides is one or more 2A peptides. In some embodiments, the one or more 2A peptides is a T2A peptide, a P2A peptide, an E2A peptide or a F2A peptide. In some embodiments, the reporter polypeptide further comprises one or more nucleic acids encoding a peptide linker between one or more of the porter and one or more of the self-cleaving peptides. In some embodiments, the peptide linker c is e sequence Gly-Ser-Gly.

In some embodiments, the reporter polypeptide is a fluorescent reporter polypeptide. In some embodiments, the reporter polypeptide for each cistron is selected from GFP, EGFP, Emerald, Citrine, Venus, mOrange, mCherry, TagBFP, mTurquoise, Cerulean, UnaG, dsRed, eqFP611, Dronpa, RFP, TagRFPs, TdTomato, KFP, EosFP, Dendra, IrisFP, IRFPs and smURFP.

In some embodiments, the open reading frame comprises a first cistron and a second cistron, where ach cistron comprises 5′ to 3′ nucleic acid comprising a MCS, nucleic acid encoding a reporter polypeptide, and nucleic acid encoding a linker peptide; wherein the first cistron and the separated by nucleic acid encoding a viral cleavage peptide. In some embodiments, the open reading frame comprises a first cistron, a second cistron and a third cistron, wherein each cistron comprises 5′ to 3′ nucleic acid comprising a MCS, nucleic acid encoding a reporter polypeptide, and nucleic acid coding a linker peptide; wherein the first cistron and the second cistron are separated by nucleic acid encoding a first viral cleavage peptide and the second cistron and the third cistron are separated by nucleic acid encoding a second viral cleavage peptide. In some embodiments, the open reading frame comprises a first cistron, a second cistron, a third cistron and a fourth cistron, wherein each cistron comprises 5′ to 3′ nucleic acid comprising a MCS, nucleic acid encoding a reporter polypeptide nucleic acid encoding a linker peptide; wherein the first cistron and the second cistron are separated by nucleic acid encoding a first viral cleavage peptide, the second cistron and the third cistron are separated by nucleic acid encoding a second viral cleavage peptide the third cistron and the fourth cistron are separated by nucleic acid encoding a third viral cleavage peptide. In some embodiments, the vector comprises a promoter operably linked to an open reading fame, wherein the open reading frame comprises a first cistron, a second cistron, a third cistron and a fourth cistron, wherein each cistron comprises 5′ to 3′ nucleic acid comprising a MCS, nucleic acid encoding a reporter polypeptide, nucleic acid encoding a linker peptide; wherein the first cistron and the second cistron are separated by nucleic acid encoding a first viral cleavage peptide, the second cistron and the third cistron are separated by nucleic acid encoding a second viral cleavage peptide the third cistron and the fourth cistron are separated by nucleic acid encoding an IRES.

In some embodiments, wherein the lineage-specific promoter is specific for cells of heart, blood, muscle, lung, liver, kidney, pancreas, brain, or skin lineage. In some embodiments, the lineage specific promoter is a sublineage-specific promoter. In some embodiments, the lineage-specific promoter is a cardiac specific promoter. In some embodiments, the cardiac-specific promoter is a MCLV2v, a SLN, a SHOX2, a MYBPC3, a TNNI3 or an α-MHC promoter. In some embodiments, the lineage-specific promoter is a neural specific promoter. In some embodiments, the neural-specific promoter is a vGAT, a TH, a GFAP, or a vGLUT1 promoter.

In some embodiments, nucleic acid encoding one or more poly ides is inserted in frame into the one or more MCS. In some embodiments, at least one cistron comprises nucleic acid encoding an organelle specific polypeptide. In some embodiments, the organelle-specific polypeptide is H2B. In some embodiments, at least one cistron comprises nucleic acid encoding an organelle marker. In some embodiments, the organelle marker comprises H2B, α-actinin 2 or a mitochondrial targeting signal fused to the reporter polypeptide.

In some embodiments, the invention provides a multireporter stem cell as described herein, wherein the one or more polypeptides comprise polypeptides that can be used to profile or distinguish a single or multiple biological pathways, cross-talk between two or more biological pathways, synthetic lethality, cellular homeostasis, organelle homeostasis, cell-cell interactions, a toxicity response or other cellular or subcellular phenotypes after differentiation of the stem cell. In some embodiments, wherein the profile is performed on a single cell. In some embodiments, wherein the reporter polypeptide can be visualized by microscopy, high throughput microscopy, fluoresence-activated cell sorting (FACS), luminescence, of using a plate reader. In some embodiments, the reporter polypeptide is analyzed before, during or after differentiation of the stem cell.

In some embodiments, the multicistronic reporter construct is integrated at a first specific cite in the genome of the multireporter stem cell. In some embodiments, the multireporter stem cell of the invention further comprises a nucleic acid integrated at a second specific cite in the genome of the multireporter stem cell. In some embodiments, the nucleic acid integrated at the second specific cite in the genome of the multireporter stem cell encodes a polypeptide, a reporter polypeptide, a cytotoxic polypeptide, a selectable polypeptide, a constitutive Cas9 expression vector or inducible Cas9 expression vector.

In some aspects, the invention provides a library of multireporter vectors, wherein the library comprises two or more multicistronic reporter vectors as described herein, wherein the two or more multicistronic reporter vectors comprise different transgenes fused to reporter polypeptides, wherein two or more of the different transgenes on each vector are expressed at essentially 1:1 stoichiometry when introduced to cells. In some aspects, the invention provides a library of multireporter vectors, wherein the library comprises two or more multicistronic reporter vectors as described herein, wherein the two or more multicistronic reporter vectors comprise different lineage-specific promoters operably linked to transgenes fused to different reporter polypeptides such that expression of the reporter polypeptides can distinguish the cell type based on the lineage specific promoter. In some embodiments, the same transgene is operably linked to the different lineages specific promoters and different reporter polypeptides. In some embodiments, the transgene encodes a housekeeping polypeptide and/or an organelle-specific polypeptide. In some embodiments, the transgene encodes H2B, α-actinin 2 or a mitochondrial targeting signal.

In some embodiments, the invention provide a library or multireporter vectors as described herein, wherein the reporter vectors encode one or more transgenes that can be used to profile or distinguish a single or multiple biological pathways, cross-talk between two or more biological pathways, synthetic lethality, cellular homeostasis, organelle homeostasis, cell-cell interactions, a toxicity response or other cellular or subcellular phenotypes after differentiation of the cell.

In some embodiments, the biological pathway or phenotype is a pathway or phenotype associated with a disease. In some embodiments, the disease is cancer, a cardiovascular disease, a neurodegenerative or neurological disease or an autoimmune disease. In some embodiments, the biological pathway or phenotype is a pathway or phenotype associated with toxic response mechanism within the cell. In some embodiments, the biological pathway or phenotype is a pathway or phenotype associated with aging. In some embodiments, the biological pathway is a pathway associated with cell proliferation, cell differentiation, cell survival cell death, apoptosis, autophagy, DNA damage and repair, oxidative stress, chromatin/epigenetics, MAPK signaling, PI3K/Akt signaling, protein synthesis, translational control, protein degradation, cell cycle and checkpoint control, cellular metabolism, development and differentiation signaling, immunology and inflammation signaling, tyrosine kinase signaling, vesicle trafficking, cytoskeletal regulation, ubiquitin pathway.

In some embodiments, the invention provides a library of multireporter cells, wherein each cell in the library comprises a multicistronic reporter vector as described herein, wherein cells in the library comprise different multicistronic reporter vectors. In some embodiments, each multicistronic reporter vector comprises a common transgene fused to a common reporter polypeptide operably linked to a common lineage specific promoter. In some embodiments, each multicistronic reporter vector comprises a common transgene fused to a different reporter polypeptide and operably linked to a different lineage specific promoter.

In some embodiments, the invention provides a library of multireporter cells comprising two or more multireporter cells as described herein, wherein two or more multireporter cells in the library comprise different multicistronic reporter vectors.

In some embodiments, the library comprises pluripotent, multipotent and/or progenitor cells. In some embodiments, the library comprises different pluripotent, multipotent and/or progenitor cells. In some embodiments, the pluripotent or multipotent cells include one or more of an induced pluripotent stem cell, a multipotent cell, a hematopoietic cell, an endothelial progenitor acceptor cell, a mesenchymal progenitor cell, a neural progenitor cell, an osteochondral progenitor cell, a lymphoid progenitor cell or a pancreatic progenitor cell. In some embodiments, the pluripotent or multipotent cells multireporter cells are differentiated after introduction of the multicistronic reporter vector. In some embodiments, different multicistronic reporter vectors were introduced to isogenic acceptor cells.

In some embodiments, the invention provides a library of multireporter cells as described herein, wherein the pluripotent or multipotent cells are differentiated after introduction of the multicistronic reporter vector. In some embodiments, different multicistronic reporter vectors were introduced to isogenic pluripotent or multipotent acceptor cells. In some embodiments, the multicistronic reporter vectors encode one or move transgenes that can be used to profile or distinguish a single or multiple biological pathways, cross-talk between two or more biological pathways, synthetic lethality, cellular homeostasis, organelle homeostasis, cell-cell interactions, a toxicity response or other cellular or subcellular phenotypes and wherein expression of the transgene operably linked to the lineage-specific promoter is used to identify the cell type or the stage of differentiation. In some embodiments, the biological pathway or phenotype is a pathway or phenotype associated with a disease. In some embodiments, the disease is cancer, a cardiovascular disease, a neurodegenerative or neurological disease or an autoimmune disease. In some embodiments, the biological pathway or phenotype is a pathway or phenotype associated with toxic response mechanism within the cell. In some embodiments, the biological pathway or phenotype is a pathway or phenotype associated with aging. In some embodiments, the biological pathway is a pathway associated with cell proliferation, cell differentiation, cell survival, cell death, apoptosis, autophagy, DNA damage and repair, oxidative stress, chromatin/epigenetics, MAPK signaling, PI3K/Akt signaling, protein synthesis, translational control, protein degradation, cell cycle and checkpoint control, cellular metabolism, development and differentiation signaling, immunology and inflammation signaling, tyrosine kinase signaling, vesicle trafficking, cytoskeletal regulation or ubiquitin pathway.

In some embodiment, the library of the invention comprises cells of two or more different lineages. In some embodiments, the cells of different lineages comprise lineage-specific reporter polypeptides.

In some aspects, the invention provides a kit comprising one or more multicistronic reporter vectors as described herein. In some embodiments, the invention provides a kit comprising one or more multireporter stem cells as described herein. In some embodiments, the kit comprises a library of multicistronic reporter stem cells arrayed in a multiwell plate. In some embodiments, the stem cells in the multiwell plate are cryopreserved.

In some aspects, the invention provides a method of profiling two or more polypeptides in a live cell, the method comprising determining the expression and/or location of the two or more of the transgenes of a multireporter stem cell as described herein. In some embodiments, the profiling is performed before, during or after differentiation of the stem cell. In some embodiments, the method is used to profile or distinguish a single or multiple biological pathways, cross-talk between two or more biological pathways, synthetic lethality, cellular homeostasis, organelle homeostasis, cell-cell interactions, a toxicity response or other cellular or subcellular phenotypes.

In some embodiments, the expression and/or location of the two or more of the transgenes is determined at one or more time points. In some embodiments, the expression and/or location of the two or more of the transgenes is determined at one or more of 1 minute, 5 minutes, 10 minutes, 15 minutes, 30 minutes, 45 minutes, 1 hour, 2 hours, 3 hours, 4 hours, 5 hours, 6 hours, 12 hours, 24 hours, 2 days, 4 days, 7 days, 14 days, 21 days, 30 days, 1 month, 3 month, 6 month, 9 month, 1 year, or more than 1 year.

In some embodiments, the invention provides a method of measuring the effects of an agent on the profile of two or more polypeptides in a live cell, the method comprising subjecting a multireporter stem cell as described herein to the agent and determining the expression and/or location of the two or more transgenes in the cell in response to the agent. In some embodiments, the profiling is performed before, during or after differentiation of the stem cell. In some embodiments, the agent is a drug or drug candidate. In some embodiments, the agent is a cancer drug or cancer drug agent. In some embodiments, the method is a toxicology screen. In some embodiments, determining the expression and/or location of the two or more transgenes is performed in a library of multireporter cells. In some embodiments, the lineage of cells in the library is determined by expression of the reporter polypeptide under the control of the lineage-specific reporter. In some embodiments, the profile is obtained using a single cell. In some embodiments, the lineage of the single cell is determined by expression of the reporter polypeptide under the control of the lineage-specific reporter.

In some embodiments, the invention provides a method of measuring the effects of an agent on the profile of two or mon polypeptides in a pool of live cells of different lineages, the method comprising subjecting a pool of multireporter stem cell as described herein to the agent and determining the expression and/or location of the two or more transgenes in the cell in response to the agent. In some embodiments, the profiling is performed before, during or after differentiation of the stem cells. In some embodiments, the agent is a drug or drug candidate. In some embodiments, the agent is a cancer drug or cancer drug agent. In some embodiments, the method is a toxicology screen. In some embodiments, determining the expression and/or location of the two or more transgenes is performed in a library of multireporter cells. In some embodiments, the lineage of cells in the library is determined by expression of the reporter polypeptide under the control of the lineage-specific reporter. In some embodiments, the profile is obtained using a single cell. In some embodiments, the lineage of cells is determined by expression of the reporter polypeptide under the control of the lineage-specific reporter.

In some embodiments, the expression and/or location of the two or more transgenes is measured by microscopy, high throughput microscopy, fluorescence-activated cell sorting (FACS), luminescence, using a plate reader, mass spectrometry, or deep sequencing.

In some aspects, the invention an acceptor cell for receiving a multicistronic reporter vector, wherein the acceptor cell comprises a recombinant nucleic acid integrated into a specific site in a host cell genome, wherein the recombinant nucleic acid comprises a first promoter operably linked to nucleic acid encoding a fusion polypeptide, wherein the fusion polypeptide comprises a reporter domain and a selectable marker domain, and wherein the nucleic acid comprises two site-specific recombinase nucleic acid sequence located at the 5′ end of the nucleic acid encoding the fusion polypeptide. In some embodiments, the nucleic acid comprises two ATG sequences located 5′ to the two specific recombinase nucleic acid sequences. In some embodiments, the promoter is a constitutive promoter. In some embodiments, the constitutive promoter is a CMV promoter, a TK promoter, an cFI-alpha promoter, a UbC promoter, a PGK promoter, a CAG promoter, an SV40 promoter, or a human β-actin promoter. In some embodiments, the site-specific recombinase sequence is a FRT nucleic acid sequence and/or an attP nucleic acid sequence and/or a loxP nucleic acid sequence. In some embodiments, the site-specific recombinase sequences comprise a PhiC31 attP nucleic acid sequence and a Bxb1 attP nucleic acid sequence. In some embodiments, the reporter domain of the fusion polypeptide is a fluorescent reporter domain. In some embodiments, the fluorescent reporter domain is selected from GFP, EGFP, Emerald, Citrine, Venus, mOrange, mCherry, TagBFP, mTurquoise, Cerulean, UnaG, dsRed, eqFP611, Dronpa, RFP, TagRFPs, TdTomato, KFP, EosFP, Dendra, IrisFP, iRFPs and smURFP. In some embodiments, the reporter domain of the fusion polypeptide is an mCherry reporter domain. In some embodiments, the selectable marker domain of the fusion polypeptide confers resistance to hygromycin, Zeocin®, puromycin, blasticidin, neomycin or an analog of hygromycin, Zeocin®, puromycin, blasticidin, neomycin. In some embodiments, the promoter is a human β-actin promoter or a CAG promoter. In some embodiments, the recombinant nucleic acid is integrated in an adeno-associated virus S1 (AAVS1) locus, a chemokine (CC motif) receptor 5 (CCR5) locus, a human ortholog of the mouse ROSA26 locus, a hip11 (H11) locus or the citrate lyase beta like gene locus (CLYBL). In some embodiments, the cell is a pluripotent cell, an induced pluripotent stem cell, or a multipotent cell. In some embodiments, the induced pluripotent stem cell is a WTC-11 cell or a NCRM5 cell. In some embodiments, the cell is a primary cell. In some embodiments, the cell is an immortalized cell. In some embodiments, the immortalized cell is a HEK293T cell, an A549 cell, an U2OS cell, an RPE cell, an NPC1 cell, a MCF7 cell, a HepG2 cell, a HaCat cell, a TK6 cell, an A375 cell or a HeLa cell.

In some embodiments of the inventions, the acceptor cell comprises a first recombinant nucleic acid for receiving a first multicistronic reporter vector and a second recombinant nucleic acid for receiving a second expression construct, wherein the first recombinant nucleic acid is integrated into a first specific site in a host cell genome and the second recombinant nucleic acid is integrated into a second specific site in a host cell genome. In some embodiments, the second recombinant nucleic acid encodes a polypeptide, a reporter polypeptide, a cytotoxic polypeptide, a selectable polypeptide, a constitutive Cas expression vector, an inducible Cas expression vector, a constitutive Cas9 expression vector or inducible Cas9 expression vector. In some embodiments, a reporter cell prepared from the acceptor cell, wherein a multicistronic reporter vector is integrated into the first specific site and a constitutive or inducible Cas expression vector (e.g., (Cas9 expression vector) is integrated into a second specific site. In some embodiments, the invention provides a method wherein a reporter cell as described herein is arrayed in a multiwell plate and used as the basis for a screen using single or oligo pool sgRNAs.

In some aspects, the invention provides a method for generating an acceptor cell for receiving a multicistronic reporter vector, the method comprising introducing a recombinant nucleic acid to a cell wherein the recombinant nucleic acid comprising 5′ to 3′ a first nucleic acid for targeting homologous recombination to a specific site in the cell, a first promoter, two ATG sequences, two site-specific recombinase nucleic acid, nucleic acid encoding a first reporter polypeptide and a selectable marker, a second nucleic acid for targeting homologous recombination to a specific site in the cell, a second promoter and nucleic acid encoding a second reporter polypeptide or a cytotoxic polypeptide, wherein expression of the first reporter polypeptide without expression of the second reporter polypeptide or cytotoxic polypeptide indicates targeting integration of the recombinant nucleic acid to the specific site in the cellular genome and expression of the first and second reporter polypeptides or a cytotoxic polypeptide indicates random integration in the cellular genome. In some embodiments, the recombinant nucleic acid is integrated into the genome of the cell using: a) an RNA guided recombination system comprising a nuclease and a guide RNA, b) a TALEN endonuclease, or c) a ZFN endonuclease. In some embodiments, cells expressing the first reporter polypeptide but not expressing the second reporter polypeptide or a cytotoxic polypeptide are selected. In some embodiments, the site-specific recombinase nucleic acids comprise a FRT nucleic acid sequence and/or an attP nucleic acid sequence and/or a loxP nucleic acid sequence. In some embodiments, the first reporter polypeptide is fluorescent polypeptide and the second reporter polypeptide is a different fluorescent polypeptide. In some embodiments, the first and second reporter polypeptide is selected from GFP, EGFP, Emerald, Citrine, Venus, mOrange, mCherry, TagBFP, mTurquoise, Cerulean, UnaG, dsRed, eqFP611, Dronpa, RFP, TagRFPs, TdTomato, KFP, EosFP, Dendra, IrisFP, iRFPs and smURFP. In some embodiments, the first reporter polypeptide is an mCherry reporter and the second reporter polypeptide is GFP. In some embodiments, the first reporter polypeptide is fluorescent polypeptide and the second reporter polypeptide is a cytotoxic polypeptide. In some embodiments, the first is selected from GFP. EGFP, Emerald, Citrine, Venus, mOrange, mCherry, TagBFP, mTurquoise, Cerulean, UnaG, dsRed, eqFP611, Dronpa, RFP, TagRFPs, TdTomato, KFP, EosFP, Dendra, IrisFP, iRFPs and smURFP and the cytotoxic polypeptide is selected from thymidine kinase (TK, e.g., HSV TK) or diphtheria toxin A (DTA). In some embodiments, the first reporter polypeptide is an mCherry reporter and the cytotoxic polypeptide is HSV TK or DTA. In some embodiments, the selectable marker confers resistance to hygromycin, Zeocin®, puromycin, blasticidin, neomycin or an analog of hygromycin, Zeocin®, puromycin, blasticidin, neomycin. In some embodiments, the first promoter is a CMV promoter, a TK promoter, an eF1-alpha promoter, a UbC promoter, a PGK promoter, a CAG promoter, an SV40 promoter, or a human β-actin promoter and the second promoter is a CMV promoter, a TK promoter, an eF1-alpha promoter, a UbC promoter, a PGK promoter, a CAG promoter, an SV40 promoter, or a human β-actin promoter. In some embodiments, the first nucleic acid for targeting homologous recombination and the second nucleic acid for targeting homologous recombination target recombination to an AAVS1 locus, a CCR5 locus, a human ortholog of the mouse ROSA26 locus, a H11 locus or a CLYBL locus. In some embodiments, the cell is an immortalized cell. In some embodiments, the immortalized cell is a HEK293T cell, an A549 cell, an U2OS cell, an RPE cell, an NPC1 cell, a MCF7 cell, a HepG2 cell, a HaCat cell, a TK6 cell, an A375 cell or a HeLa cell. In some embodiments, the cell is a pluripotent cell, an induced pluripotent stem cell, or a multipotent cell. In some embodiments, the induced pluripotent stem cell is a WTC-11 cell or a NCRM5 cell. In some embodiments, the cell is a primary cell.

In some embodiments, the method of preparing an acceptor cell further comprises introducing a second recombinant nucleic acid to a cell for receiving a second multicistronic reporter vector wherein the second recombinant nucleic acid comprises 5′ to 3′ a third nucleic acid for targeting homologous recombination to a specific site in the cell, a third promoter, two ATG sequences, two site-specific recombinase nucleic acid, nucleic acid encoding a third reporter polypeptide and a selectable marker, a fourth nucleic acid for targeting homologous recombination to a specific site in the cell, a fourth promoter and nucleic acid encoding a fourth reporter polypeptide or cytotoxic (e.g., killer) polypeptide, wherein expression of the third reporter polypeptide without expression of the fourth reporter or cytotoxic polypeptide indicates targeting integration of the recombinant nucleic acid to the specific site in the cellular genome and expression of the third and fourth reporter or cytotoxic polypeptides indicates random integration in the cellular genome.

### BRIEF DESCRIPTION OF THE DRAWINGS

FIG. 1A shows a multicistronic vector that can express up to 4 transcripts from a single promoter. The platform contains an attB Bxb1-specific site for integration of the reporters in the acceptor site shown in A. Our platform is configured to enable plug-and-play insertion/swapping of promoters, resistance markers, fluorescent labels and proteins of interest. FIG. 1B shows transient expression of MTS-mVenus (mitochondria) and TagBFP-H2B (DNA/nucleus) in WTC hiPSC lines using the multicistronic platform demonstrates correct and separate localization of these reporters. FIG. 1C shows hiPSC WTC acceptor cells stably express a TagBFP control reporter recombined at the acceptor site. Images show WTC^{A} clone 18 after recombination of the control TagBFP reporter and antibiotic selection. Loss of cytoplasmic mCherry fluorescence in cells expressing TagBFP confirms stable recombination of the control reporter at the acceptor site (example labeled with arrow). Cells that do not express TagBFP retain cytoplasmic mCherry expression (example labeled with arrowhead). FIG. 1D depicts the new acceptor site design for AAVS1 targeting in hiPSCs which contains: a CAG constitutive promoter, 2 alternative ATGs in different frames each set to drive expression of the fluorophore fused to the resistant marker after recombination with the multicistronic vectors, an attP site to access recombination by PhiC31, an attP site to access recombination by BxB1, mCherry fluorescence marker fused to puromycin gene, and CMV-GFP located after the AAVS1-Right homology arm (AAVS1-R). GFP allows for differentiation between random and targeted integrations. Cells with random integration fluoresce green due to GFP expression, while cells with targeted integration do not fluoresce green due to loss of CMV-GFP since it's outside the acceptor site region integrated into cells (between AAVS1-L and AAVS1-R). FIG. 1E shows representative examples of iPSC WTC acceptor cell after recombination of the optimized acceptor site containing PhiC31 only (Top mw) or PhiC31Bxb1 sites (Bottom row). Images show WTC after integration of the AAVS1 acceptor site and antibiotic selection. Cells express mCherry revealing that integration of the acceptor site occurred and cells do not express GFP meaning that no random integration occurred.

FIG. 2A represents the iPSC acceptor cell line and 3 cardio lineage specific reporter cell lines derived from it Once the cells are differentiated they will express different labelled markers depending which lineage they are: ventricular, atrial or nodal. FIG. 2B depicts multicistronic constructs for cardiomyocyte lineage specific expression. CM-Functional depicts multicistronic constructs to achieve lineage-specific nuclear barcoding in ventricular, atrial and nodal hiPSC-CMs to monitor functional alterations in CMs cells Three different strategies to monitor CM structural alteration were assembled: the constructs carry lineage-specific nuclear barcoding with simultaneous expression of 3 labeled cellular structures (nuclei/DNA, H2B: mitochondria, MTS; and sarcomeres, ACTN) (FP—fluorescent protein) CM-Structural-1—minimal vector size configuration; CM-Structural-2—increased promoter activity configuration; CM-Structural-3—increased expression configuration. Cardiomyocyte lineage-specific barcoding constnucts and 2 control constricts for general CM expression and for expression in undifferentiated hiPSCs.

FIGS. 3A and 3B show iPSC-derived cardiomyocytes transiently transfected with CM-Structural-1 carrying fluorescently tagged MTS (mitochondria). ACTN2 (Actinin) and H2B (nucleus) under the control of the constitutive promoter CAGGS (FIG. 3A) or 3 cardiomyocyte lineage specific promoters (MLC2v, SHOX2 and SLN) FIG. 3D in WTC-11 hiPSC-derived cardiomyocytes demonstrating expression of these reporters. Scale bar 10 μm.

FIGS. 4A and 4B depict the increased expression of the CM-Tox2 system relatively to CM-Tox1 to drive expression of a single reporter when using CM-lineage specific promoters Representative images of iPSC-derived cardiomyocytes transiently transfected with CM-Structural 1 or 2 visualized 4 days after transfection. FIG. 4A iPSC-derived CMs transfected with CM-Tox1 (left) and CM-Tox2 (right) driven by constitutive promoter CAGGS do not show any significative different in terms of number of transfected cells or levels of expression. FIG. 4B iPSC-derived CMs transfected with CM-Tox1 (left) and CM-Tox2 (right) driven by nodal lineage specific promoter, SHOX2 in this case there is a clear increase in both number of transfected cells and levels of expression of H2B-Venus. Scale bar 10 μm.

FIG. 5 shows iPSC-derived cardiomyocytes transiently transfected with CM-Structural-2, which uses the tTA-TRE system to drive expression of tagged MTS (mitochondria), ACTN2 (Actinin) and H28 (nucleus) under the control 3 cardiomyocyte lineage specific promoters (MLC2v, SHOX2 and SLN) in WTC-11 hiPSC-derived cardiomyocytes demonstrating correct localization of these reporters. Scale bar 10 μm.

FIG. 6A depicts immunolabeling of cultured iPSC-derived cardiomyocytes for 2 cardiac markers. Day-15 cardiomyocytes derived from WTC iPSCs were labeled with antibodies against the cardiac markers α-actinin and cardiac Troponin T and examined by microscopy. Representative images show a pattern characteristic of sarcomere organization (arrowheads). FIG. 6B Flow cytometry of iPSC-derived cardiomyocytes. Cardiomyocytes derived from WTC (day−26) and NCRM-5 (day−16) iPSCs were labeled with an antibody against the cardiac marker cardiac Troponin T and examined by flow cytometry (grey histogram). Control samples were labeled with the same isotype primary antibody (black histogram). Populations and percentages of cells positive for cTnT are denoted by the brackets (labeled “anti-cTnT+”).

FIG. 7A represents the iPSC acceptor cell line and 4 neuro lineage specific reporter cell lines derived from it. Once the cells are differentiated they will express different labelled markers depending which lineage they are: GABAergic, Dopaminergic, Glutamatergic and astrocytes. FIG. 7B depicts multicistronic constructs for neural lineage specific expression (A) Constructs for lineage specific to achieve lineage-specific nuclear barcoding in GABAergic, Dopaminergic, Glutamatergic and astrocytes hiPSC. Three different strategies to assemble constructs to achieve lineage-specific nuclear barcoding with simultaneous expression of 3 labeled cellular structures (nuclei/DNA, H2B; mitochondria, MTS; and membrane, palm) (FP—fluorescent protein). NP-Tox1—increased promoter activity configuration; NP-Tox2—minimal vector size configuration; NP-Tox3—increased expression configuration. Neural lineage-specific barcoding constructs and control construct for general expression in undifferentiated hiPSCs.

FIG. 8 depicts the plug-and-play acceptor site design. The customizable design (indicated with dashed and non-dashed lines) allows for choice of 1) locus of integration; 2) integrase to use for recombination of reporter construct; 3) fluorophore and selection marker and 4) negative or fluorescence random integration marker. The acceptor site design contains: a CAG constitutive promoter, 2 alternative ATGs in different frames, an attP site to access recombination by PhiC31, an attP site to access recombination by BxB1, mCherry fluorescence marker fused to puromycin gene (or TagBFP fused to Zeocin resistance gene (Zeocin)) or blasticidin), and option of a negative selection marker (diphtheria toxin-A (DT-A) or herpes simplex virus thymidine kinase (HSV-TK)) or a random integration GFP marker located after the AAVS1-Right homology arm (AAVS1-R). HSV-TK allows for selective suicidal effect of cells with random plasmid integration after exposure of cells to ganciclovir (GCV), a guanosine analog. HSV-TK phosphorylates GCV to GCV-monophosphate which is further converted to GCV-diphosphate and GCV-triphosphate by host kinases GCV-triphosphate causes premature DNA chain termination and apoptosis. On the other hand, DTA is a fragment of the diphtheria toxin that once expressed in cells inhibits protein synthesis leading to cell death. GFP allows for differentiation between random and targeted integrations. Cells with random integration fluoresce green due to GFP expression, while cells with targeted integration do not fluoresce green due to loss of CMV-GFP since it's outside the acceptor site region integrated into cells (between AAVS1-L and AAVS1-R or between H11-L and H11-R indicated with black line). These random integration markers can be driven by CMV, CAG or eiFalpha constitutive promoters. Dashed and non-dashed line boxes indicate regions that can be permutated, grey shaded box indicate regions to be present in each plasmid to be integrated in different genomic loci.

### DETAILED DESCRIPTION

The present invention provides stem cell lineage-specific labeling that can profile individual cell lineages in heterogeneous populations of stem cell-derived differentiated live cells (e.g., in single live cells). In some aspects, the invention provides a multicistronic reporter vector comprising: a promoter operably linked to an open reading frame, wherein the promoter is a lineage-specific promoter, wherein the open reading frame comprises two or more cistrons, and wherein expression of the open reading frame in a cell yields separate component polypeptide products from each cistron; wherein each cistron comprises a multiple cloning site (MCS) and nucleic acid encoding a reporter vector, wherein each cistron encodes a different reporter polypeptide; and wherein expression of two or more nucleic acids encoding polypeptides inserted into the Iwo or more multiple cloning sites and fused to the reporter polypeptides is essentially at about 1:1 stoichiometry.

In some aspects, the invention provides a multicistronic reporter vector comprising: a first promoter linked to a transactivator polypeptide, wherein the first promoter is a lineage-specific promoter, a second promoter operably linked to an open rending frame, wherein the second promoter is inducible by the transactivator polypeptide, wherein the open reading frame comprises two or more cistrons, and wherein expression of the open reading frame in a cell yields separate component polypeptide products from each cistron; wherein each cistron comprises a multiple cloning site (MCS) and nucleic acid encoding a reporter vector, wherein each cistron encodes a different reporter polypeptide; and wherein expression of two or more nucleic acids encoding polypeptides inserted into the two or more multiple cloning sites and fused to the reporter polypeptides is essentially at about 1:1 stoichiometry. In some embodiments, the transactivator polypeptide is a tetracycline transactivator polypeptide and the second promoter comprises a tetracycline responsive element. In some embodiments, the tetracycline responsive element is a Tet operator 2 (TetO2) inducible or repressor element.

In some aspects, the invention provides a multicistronic reporter vector comprising a first promoter linked to a nucleic acid encoding a nuclear polypeptide, wherein the First promoter is a lineage-specific promoter; a second promoter operably linked to an open reading frame, wherein the second promoter is a constitutive promoter, wherein the open reading frame comprises two or more cistrons, and wherein expression of the open reading frame in a cell yields separate component polypeptide products front each cistron; wherein each cistron comprises a multiple cloning site (MCS) and nucleic acid encoding a reporter vector, wherein each cistron encodes a different reporter polypeptide, and wherein expression of two or more nucleic acids encoding polypeptides inserted into the two or more multiple cloning sites and fused to the reporter polypeptides is essentially at about 1:1 stoichiometry. In some embodiments, the housekeeping polypeptide is H2B.

In some embodiments, the invention provides a multicistronic reporter vector comprising: a promoter operably linked to an open reading frame, wherein the open reading frame comprises two or more cistrons, and wherein expression of the open reading frame in a cell yields separate component polypeptide products from each cistron: wherein each cistron comprises a multiple cloning site (MCS) and nucleic acid encoding a reporter vector, wherein each cistron encodes a different reporter polypeptide; and wherein expression of two or more nucleic acids encoding polypeptides inserted into the two or more multiple cloning sites and fused to the reporter polypeptides is essentially 1:1 stoichiometric. In some aspects, the invention provides acceptor cells for receiving the multicistronic reporter vectors. In yet other aspects, the invention provides multireporter cells for multiplex high content assays wherein the multireporter cells comprise any of the multicistronic reporter vectors described herein. The multireporter cells described herein may be used in live cell assays to profile the expression and activity of multiple polypeptides in live cells (e.g., single live cells) as a means to profile or distinguish aspects of cell behavior including, but not limited to, individual or multiple biological pathways, cross-talk between two or more biological pathways, synthetic lethality, cellular homeostasis, organelle homeostasis, other cellular or subcellular phenotypes, cell-cell interactions aspects of cell behavior including, but not limited to, biological pathways, cross-talk between biological pathways, cellular homeostasis, organelle homeostasis and toxicity and perturbations to these behaviors that may be induced by a candidate therapeutic or other chemical compound or other stimuli or combinations thereof.

High-throughput live cell microscopy-based screening offers the opportunity to screen compounds in cellular systems that recapitulate the dynamic nature of signal transduction and cellular phenotypes that is not captured by end point assays. Stem cells or human induced pluripotent stem cells (iPSCs) have great potential as cellular models used in live cell screening by providing physiological relevance and high reproducibility in a format scalable to high-throughput applications.

Described herein are novel methods, stem cells with barcoded lineages and multiplexed high-throughput assays that provide mechanistic and phenotypic readouts of cellular stress, homeostasis, and related events in stem cells, which have the potential of being differentiated into a variety of cell types in vitro. A wide variety of chemicals are known to perturb homeostasis and cause cellular stress, and it is thus an important aspect of cellular physiology to monitor in the context of understanding mechanisms of activity or toxicity mechanisms of candidate therapeutics or other chemical or genetic perturbations. The methods described herein may be used to interrogate the mechanism of action and any potential collateral cytotoxicity of therapeutic agents as well as potential toxicity of other chemicals such as industrial and environmental chemicals and the ability to profile biological effects of genetic manipulation.

In some embodiments, the methods, cells and multiplexed high-throughput assays are used to profile cardiotoxicity. Cardiotoxicity liabilities result in the failure of approximately one third of therapeutic compounds entering Phase I clinical trials across therapeutic indications. Thus, improved methods to profile cardiotoxicity signals early in the drug discovery process will serve to improve the efficiency and success rates of compounds entering clinical trials.

Cardiotoxicity represents a detrimental side effect of cancer treatment, resulting in considerable morbidity and mortality. Cytotoxic agents and targeted therapies used to treat cancer, including classic chemotherapeutic agents, antibodies and small molecule tyrosine kinase Inhibitors, and chemoprevention agents all affect the cardiovascular system and may result in severe effects such as heart failure, ventricular dysfunction, and myocardial ischemia. The rise in cancer therapy-induced cardiomyopathies suggests that the risks of cardiotoxicity must be carefully weighed during the evaluation and development of any anti-cancer drug.

The molecular mechanisms linking cancer therapies to cardiomyopathies, including the specific contribution of stress-induced transcription factors to cell survival or death, are not well understood due to the lack of a system for real-time monitoring of this aspect of toxicology in biologically-relevant cardiac cells. The assays described herein address this need through provision of multiplexed fluorescent reporter systems that provide readout of cellular stress and organelle homeostasis using human stem cell-derived reporter cardiomyocytes. The effects of molecules and potential therapeutic agents for cancer and other diseases can also be assessed for neuro toxicity, developmental toxicity, hepatic toxicity, or any other type of tissue toxicity by adapting this approach to other IPS cell-derived lineages and sublineages.

Furthermore, the molecular mechanisms that underpin the high failure rates due to cardiotoxicity in Phase I clinical trials ae also poorly understood, and the assays described herein address this through provision of multiplexed fluorescent reporter systems that provide readout of cellular stress and organelle homeostasis using human stem cell-derived reporter cardiomyocytes.

The major limitations of using primary cardiomyocytes, primary neurons or other primary cell types, are the technical difficulties associated with obtaining and maintaining these cells. For example while immortalized cardiac cells are convenient because they can readily proliferate, beat, and in some cases, stably maintain a cardiac phenotype, their metabolism and morphology may be different from cardiomyocytes so their use has been limited in toxicology studies. Cardiomyocytes derived from stem cells or iPSCs overcome these disadvantages and provide a tool to not only assess the effect of molecules on the terminally differentiated cells, but also to study development or the effect of various molecules through different stages of differentiation. This is particularly important since reliable tests on progenitor and differentiating cells is valuable but sparse. Furthermore, iPSCs can be generated from human subjects to examine a variety of diseased and normal phenotypes.

In some embodiments, the methods, stem cell and multiplexed high-throughput assays are used to profile neurotoxicity. Neurotoxicity and developmental neurotoxicity are important adverse health effects of hundreds of environmental contaminants and occupational chemicals, natural toxins and pharmaceutical drugs, leading to, for example neurological and developmental defects in children and neurological changes such as addiction in adults.

In vivo testing guidelines for neurotoxicity and developmental neurotoxicity have been developed, implemented and validated. However, such n vivo tests are time-consuming, expensive and require the use of a substantial number of animals. Neural cells derived from stem cells or iPSCs overcome these disadvantages and provide a tool to not only assess the effect of molecules on the terminally differentiated cells, but also to study development or the effect of various molecules though different stages of differentiation. This is particularly important since reliable tests on progenitor and differentiating cells is essential to profile developmental neurotoxicity. Furthermore, iPSCs can be generated from human subjects to examine a variety of diseased and normal phenotypes.

There is a need for new approaches to visualize and quantify the spatiotemporal modulation of intercellular and subcellular interactions in live stem cell-derived neuronal cells at scale to dissect the mechanisms of central nervous system (CNS) disease progression, empower the discovery of therapies for these disorders and pinpoint the neurotoxicity liabilities of chemical entities.

In some embodiments, the methods, cells and multiplexed high-throughput assays are used in toxicity profiling. For example, the methods, stem cells with barcoded lineages and multiplexed high-throughput assays are used in drug discovery to evaluate cardiotoxicity.

In some embodiments, the methods, stem cells with barcoded lineages and multiplexed high-throughput assay are used in a pooled assay Combining lineage specific labeling approach with multiple (e.g., 2, 3, 4, 5, etc.) structural fluorescent reporters that are permuted differently for each cell lineage enables development of an assay with pooled populations of different CM lineages that can be identified and monitored through their unique fluorescein barcode. This enables the development of more physiologically relevant assays with co-culture of mixed cell lineages, in contrast to assays based on purified populations of a single cell lineages which are known to exhibit altered phenotypes when isolated from other lineages. Furthermore, the pooled cardiotoxicity assays enabled by our fluorescent barcoding approach enable parallel assessment of three or four cell lineages, contrasting with the need to run separate assays for purified cell populations.

In some embodiments, the methods, stem cells with barcoded lineages and multiplexed high-throughput assay are used to identify and monitor cell lineages in the context of more advanced stem cell-derived cellular models. The use of stem cells in the development of advanced models such as 3D cultures, microfluidic-enabled ‘organ-on-a-chip’, and 3D bioprinted models is a rapidly growing area with applications in drug discovery toxicity testing and basic research. Interestingly, in spite of the increased physiological relevance and sophistication of these approaches, the tools available to characterize these models are somewhat limited and broadly comprise staining of live cultures with live cell compatible dyes and sectioning and immunofluorescence staining of fluid samples. For the full power of these sophisticated new models to be realized, new approaches are required that will enable reliable, real-time monitoring of functional, structural and mechanistic parameters.

In some embodiments, the methods, stem cells with barcoded lineages and multiplexed high-throughput assay a used to integrate structural and functional readouts with several parameter readouts. An integrated assay that measures a combined several parameters encompassing structural and functional readouts.

In some embodiments, the methods, stem cells with barcoded lineages and multiplexed high-throughput assay are used to examine developmental neurotoxicity, which has been recognized to be the cause of developmental disorders such as autism, attention deficit disorder, mental retardation or cerebral palsy by implementing a high-content screening assay that assesses the toxicity of compounds in specific neural lineages during early developmental stages.

In some embodiments, the methods, stem cells with barcoded lineages and multiplexed high-throughput assays are used in drug discovery. For example, the methods, cells and multiplexed high-throughput assays are used in drug discovery to treat neurodegeneration. In some embodiments, the invention provides the use of stem cell-derived cells in drug development to treat neurodegenerative diseases.

In some embodiments, the methods, stem cells with barcoded lineages and multiplexed high-throughput assays are used in drug discovery. For example, the methods, cells and multiplexed high-throughput assays are used in drug discovery to treat addiction. In some embodiments, the invention provides the use of stem cell-derived cells in drug development to treat addiction.

### Definitions

A “vector.” as used herein, refers to a recombinant plasmid or virus that comprises a nucleic acid to be delivered into a host cell, either in vitro or in vivo.

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the nucleic acid can comprise sugars and phosphate groups (as may typically be found in RNA or DNA), or modified or substituted sugar or phosphate groups. Alternatively, the backbone of the nucleic acid can comprise a polymer of synthetic subunits such as phosphoramidates and thus can be an oligodeoxynucleoside phosphoramidate (P—NH₂) or a mixed phosphoramidate-phosphodiester oligomer. In addition, a double-stranded nucleic acid can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-translational modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

A “biosensor” as used herein, refers to reporter compounds that are attached to an additional protein sequence that make it sensitive to small biomolecules or other physiological intracellular processes. In nonlimiting examples, the biosensor is a fluorescent biosensor including a genetically encoded fluorescent polypeptide. Biosensors are introduced into cells, tissues or organisms to allow for detection (e.g., by fluorescence microscopy) as a difference in FRET efficiency, translocation of the fluorescent protein or modulation of the reporter properties of a single reporter protein Many biosensors allow for long-term imaging and can be designed to specifically target cellular compartments or organelles. Another advantage of biosensors is that they permit investigation of a signaling pathway or measurement of a biomolecule while largely preserving spatial and temporal cellular processes.

An “acceptor cell” is a cell which has been engineered to harbor an acceptor construct in its genome.

An “acceptor construct” is sequence of nucleotide which comprises a sequence of nucleic acid which can harbor a reporter nucleic acid.

The term “transgene” refers to a nucleic acid that is introduced into a cell and is capable of being transcribed into RNA and optionally, translated and/or expressed under appropriate conditions.

As used herein “stem cell”, unless defined further, refers to any non-somatic cell. Any cell that is not a terminally differentiated or terminally committed cell may be referred to as a stem cell. This includes embryonic stem cells, induced pluripotent stem cells, hematopoietic stem cells, progenitor cells, and partially differentiated progenitor cells. Stem cells may be totipotent, pluripotent, or multipotent stem cells. Any cell which has the potential to differentiate into two different types of cells is considered a stem cell for the purpose of this application.

An “iPS” cell as used herein refers to any pluripotent cell obtained by re-programing a non-pluripotent cell. The reprogrammed cell mal have been generated by reprogramming a progenitor cell, a partially-differentiated cell, or a fully differentiated cell of any embryonic or extraembryonic tissue lineage.

“Reprogramming” as used herein refers to the process of de-differentiating a cell which is at least partially differentiated into a pluripotent state.

As used herein “immune privileged cell” refers to a cell which elicits a diminished immune response when introduced into a foreign host organism.

As used herein “cistron” refers to a segment of nucleic that is equivalent to agene and that encodes a single functional unit e.g., a single polypeptide or a fusion polypeptide comprising a transgene product and a reporter domain). As used herein, a multicistronic vector is a nucleic acid that comprises two or more cistrons. In some embodiments, the multicistronic vector comprises two or more cistrons in a single open reading frame. In some embodiments, the single open reading frame, when translated, generates two or more polypeptides that are dissociated from one another.

As used herein, the term “essentially 1:1 stoichiometric expression” with regard to expression of two or more reporter polypeptides refers to the expression of two or more reporter polypeptides wherein the expression level of the two or more reporter polypeptides are about the same. In some embodiments, the expression of the two or mom reporter polypeptides is equal or varies by no more than any of about 5%, 10%, 15%, 20% or 25% of each other.

As used herein, a “site-specific recombinase sequence” refers to a target sequence of site-specific recombination system. Site-specific recombination systems include, but are not limited to, Tyr recombinases, Ser integrases, Cre recombinases with loxP target sequences, FLP recombinase with FRT target sequence. Site-specific recombination nucleic acid sequences for Tyr recombinases and Ser integrase % (e.g., PhiC31) integrases include but are not limited to attB, and attP. Site-specific recombination nucleic acid sequences for CRE recombinase include but is not limited loxP. Site-specific recombination nucleic acid sequences for FLP recombinase include but is not limited FRT.

As used herein, a “lineage specific promoter” refers to a region of DNA that initiates transcription of a particular gene in a conditional fashion, i.e., only in a specific cell lineage. A lineage specific promoter is used to restrict expression of reporter genes and transgenes to a lineage in which the promoter is active. Some lineage specific promoters are also sublineage specific promoters.

As used herein, a “sublineage specific promoter” refers to a region of DNA that initiates transcription of a particular gene in a conditional fashion. i.e., in a specific cell sublineage. A sublineage specific promoter is used to restrict expression of reporter genes and transgenes to a sublineage in which the promoter is active.

As used herein, a “lineage barcode” refers to one or more fluorescent proteins, each operably linked to a cellular marker, under the control of a lineage or sublineage specific promoter. In some examples, the cellular marker is an organelle marker. A lineage barcode allows a cell in which the lineage or sublineage specific promoter is active to be identified based on a distinct fluorescent signature. A lineage barcode can be used in methods whereby different fluorescent proteins operably linked to a cellular marker are inserted in the genome of a cell under the control of a lineage specific or sublineage specific promoter. In some aspects, the purpose of the lineage barcode is to identify an unknown lineage in terms of a preexisting classification. Each cell lineage in a mixed population will be identifiable based on a distinct fluorescent signature.

Reference to “about” a value or parameter herein includes land describes) embodiments that are directed to that value or parameter per se. For example, description referring to “about X” includes description of “X.”

As used herein, the singular form of the articles “a,” “an,” and “the” includes plural references unless indicated otherwise.

It is understood that aspects and embodiments of the invention described herein include “comprising,” “consisting,” and/or “consisting essentially of” aspects and embodiments.

### Acceptor Cells

The present disclosure provides multireporter cells and methods for generating multireporter cells, which can be used to profile two or more polypeptides in alive stem cell and provide information on the identity of the cell via lineage specific promoters. Multireporter cells are developed by cloning a multicistronic reporter vector into an insertion site of an acceptor cell. Acceptor cells are developed by incorporating recombinant nucleic acid encoding an acceptor sequence into the genome of a cell. The acceptor sequence comprises an insertion site which allows for the site-specific integration of the multicistronic reporter vector into the acceptor cell genome. As described herein, the multicistronic reporter vector comprises nucleic acid encoding two or more polypeptides wherein the polypeptides are fused to a reporter domain. The two or more nucleic acid sequences encoding for the polypeptides of interest are located within the same open reading frame, allowing for essentially 1:1 stoichiometric expression of the recombinant peptides.

In some aspects, the invention provides an acceptor cell for receiving a multicistronic reporter vector, wherein the acceptor cell comprises a recombinant nucleic acid integrated into a specific site in a host cell genome, wherein the recombinant nucleic acid comprises a first promoter operably linked to nucleic acid encoding a fusion polypeptide, wherein the fusion polypeptide comprises a reporter domain and a selectable marker domain, and wherein the nucleic acid comprises a site-specific recombinase nucleic acid sequence located at the 5′ end of the nucleic acid encoding the fusion polypeptide.

In some embodiments, the promoter (e.g., the first promoter) is a constitutive promoter. Examples of constitutive promoters include but are not limited to a cytomegalovirus immediate early (CMV) promoter, a thymidine kinase (TK) promoter, an eF1-alpha, a Ubiquitin C (UbC), a Phosphoglycerate Kinase (PGK), a CAG promoter, an SV40 promoter, or a human β-actin promoter. In some embodiments, the promoter is an inducible promoter. Examples of inducible promoters include but are not limited to a tetracycline response promoter, a rapamycin-regulated promoter, and a sterol inducible promoter. In some embodiments, the inducible promoter is a tetracycline responsive promoter.

In some embodiments of the invention, the acceptor site comprises a site-specific recombinase sequence. Examples of site-specific recombinase sequences include but are not limited to a FRT nucleic acid sequence, an attP nucleic acid sequence and loxP nucleic acid sequence. In some embodiments, the site-specific recombinase sequence is a FRT nucleic acid. In some embodiments, the site-specific recombinase sequence is an attP nucleic acid. In some embodiments, the site-specific recombinase sequence is an attB nucleic acid. In some embodiments, the site-specific recombinase sequence is a loxP nucleic acid. In some embodiments, the site-specific recombinase sequence is a FRT nucleic acid and an attP sequence. In some embodiments, the site-specific recombinase sequence is a FRT nucleic acid and an attB sequence.

In some embodiments, the acceptor site comprises a nucleic acid encoding a reporter polypeptide. In some embodiments, the acceptor site comprises nucleic acid encoding a selection polypeptide. In some embodiments, the acceptor site comprises nucleic acid encoding a reporter polypeptide (e.g., a reporter domain) fused to a selection polypeptide (e.g., a selection domain) in some embodiments, the reporter polypeptide is on the N-terminus of the fusion polypeptide and the selection polypeptide is on the C-terminus of the fusion polypeptide. In other embodiments, the selection polypeptide is on the N-terminus of the fusion polypeptide and the reporter polypeptide is on the C-terminus of the fusion polypeptide.

A reporter peptide is a peptide which can be readily identified: for example via microscopy, plate reader, FACS, chemically, mass spectrometry, or deep sequencing. For example the reporter domain may be a fluorescent or luminescent polypeptide. In some embodiments, the reporter domain may be a green fluorescent protein (GFP) or any of its derivatives. In some embodiments the reporter domain is a non GFP derived fluorescent peptide. In some embodiments the reporter domain encodes for GFP, FGFP, Emerald, Citrine, Venus, mOrange, mCherry, TagBFP, mTurquoise, Cerulean, UnaG, dsRed, eqFP611, Dronpa, RFP, TagRFPs, TdTomato, KFP, EosFP, Dendra, IrisFP, iRFPs, or smURFP. The reporter domain may be a luciferase. The reporter domain may be an enzyme, which when expressed allows for visualization of expression through the products of a chemical reaction. In some embodiments, the reporter is a firefly luciferase or a renilla luciferase. In some embodiments the reporter domain is β-glucuronidase or β-galactosidase.

A selectable marker domain may be a polypeptide which confers resistance to a molecule the cell is not normally resistant to, or at a dose the cell is not normally resistant to. For example the selectable marker domain may be a polypeptide which confers resistance to an antibiotic. In some embodiments the selectable marker is polypeptide that confers resistance to blasticidin, geneticin, hygromycin, puromycin, neomycin, Zeocin™, kanamycin, carbenicillin, ampicillin, actinomycin, apramycin, mycophenolic acid, histidinol, methotrexate or any of their salts or derivatives.

In some embodiments, the acceptor cell comprises an acceptor site which comprises nucleic acid encoding a fusion polypeptide comprising a reporter domain and a selection domain. In some embodiments, the reporter domain of the fusion polypeptide is an mCherry reporter domain. In some embodiments, the selectable marker domain of the fusion polypeptide confers resistance to hygromycin, Zeocin™, puromycin, blasticidin, neomycin or an analog of hygromycin, Zeocin™, puromycin, blasticidin, neomycin.

In some embodiments, the acceptor site further comprises nucleic acid encoding a gene expression repressor polypeptide. In some embodiments, the acceptor site comprises nucleic acid encoding a tetracycline repressor polypeptide operably linked to a promoter. In some embodiments, the constitutive promoter is a CMV promoter, a TK promoter, an eF1-alpha, an UbC promoter, a PGK promoter, a CAG promoter, an SV40 promoter, or a human β-actin promoter. In some embodiments, the promoter is a human β-actin promoter or a CAG promoter.

The acceptor site is integrated at a specific site in the genome of the acceptor cell. In some embodiments, the specific site is an innocuous site in the acceptor cell genome. For example, insertion of a nucleic acid into the specific site has little impact on the functions of the acceptor cell. In some embodiments, the recombinant nucleic acid is integrated in an adeno-associated virus S1 (AAVS1) locus, a chemokine (CC motif) receptor 5 (CCR5) locus, a human ortholog of the mouse ROSA26 locus, the Hipp 11 (H11) locus, or the citrate lyase beta like gene locus, (CLYBL). In some embodiments, the acceptor site comprises heterologous nucleic acid sequences that were used to target the recombinant nucleic acid encoding the site-specific recombinase nucleic acid sequence to the specific target locus in the acceptor cell genome. In some embodiments, the acceptor cell comprises nucleic acid for targeting to the AAVS1 locus, the CCR5 locus, the mouse ROSA26 locus or the human ortholog of the mouse ROSA26 locus, the H11 locus, or the CLYBL locus.

The present disclosure provides methods to generate acceptor cell lines from stem cells. The method includes engineering a stem cell so that the cell can harbor a reporter nucleic acid. Any stem cell can be an acceptor cell. In some embodiments the cell used is a mammalian stem cell. In some embodiments the cell used is a human stem cell. In some embodiments the acceptor cell line is generated by engineering a primary stem cell. The primary cell may be harvested from a plant or an animal. In some embodiments the primary stem cell is harvested from a mammal. In some embodiments the primary stem cell is harvested from a human in some embodiments the primary stem cell is harvested from a rodent. In some embodiments the stem cell used is a patient specific cell.

In some embodiments the acceptor cell is a stem cell. The stem cell may be a totipotent, a pluripotent or a multipotent stem cell. Any totipotent, pluripotent, multipotent or progenitor stem cell may be used to generate an acceptor cell line. The stem cell may be an animal cell. In some embodiments the stem cell is from a mammal. In some embodiments the stem cell is from a human. In some embodiments, the stem cell is a patient specific stem cell. In some embodiments, the stem cell is an autologous stem cell. In some embodiments, the stem cell is an allogeneic stem cell. In some cases the stem cell is from a non-human primate, a dog or a rodent. The stem cell may be derived from the trophectoderm, the inner cell mass of a blastocyst, or a specific tissue. The stem cell may be an embryonic stem cell, an induced pluripotent stem cell or a progenitor stem cell. Any progenitor cell can be used to generate an acceptor cell line. For example the progenitor cell used may be a hematopoietic cell, as endothelial progenitor cell, a mesenchymal progenitor cell, a neural progenitor cell, an osteochondral progenitor cell, a lymphoid progenitor cell, a hepatic progenitor cell, or a pancreatic progenitor cell.

In some embodiments, the acceptor cell is a plant cell or an animal cell. In some embodiments the animal is an invertebrate. In some embodiments the acceptor cell is a cell from a member of the Arabidopsis genus. In some embodiments the acceptor cell is a cell from a member of the Drosophila melanogaster species. In some embodiments the acceptor cell is a cell from a member of the Caenorhabditis elegans species. In some embodiments the acceptor cell is a vertebrate animal cell. In some embodiments the acceptor cell is a mammalian cell. In some embodiments the acceptor cell is a human cell, a primate cell, a rodent cell, a feline cell, a canine cell, a bovine cell, a porcine cell or an ovine cell.

In some embodiments, the invention provides a method for generating an acceptor cell for receiving a multicistronic reporter vector, the method comprising introducing a recombinant nucleic acid to a cell wherein the recombinant nucleic acid comprising 5′ to 3′ a first nucleic acid for targeting homologous recombination to a specific site in the cell, a first promoter, site-specific recombinase nucleic acid, nucleic acid encoding a first reporter polypeptide and a selectable marker, and a second nucleic acid for targeting homologous recombination to a specific site in the cell. In some embodiments, the recombinant nucleic acid comprises any of the acceptor sites described above to generate any of the acceptor cells described above.

In some embodiments, the invention provides a method for generating an acceptor cell for receiving a multicistronic reporter vector, the method comprising introducing a recombinant nucleic acid to a cell wherein the recombinant nucleic acid comprising 5′ to 3′ a first nucleic acid for targeting homologous recombination to a specific site in the cell, a first promoter, one or two site-specific recombination nucleic acids, nucleic acid encoding a first reporter polypeptide and a selectable marker, a second nucleic acid for targeting homologous recombination to a specific sire in the cell, a second promoter and nucleic acid encoding a second reporter polypeptide or a cytotoxic polypeptide, wherein expression of the first reporter polypeptide without expression of the second reporter polypeptide or cytotoxic polypeptide indicates targeting integration of the recombinant nucleic acid to the specific site in the cellular genome and expression of the first and second reporter polypeptides or expression of the first reporter polypeptide and the cytotoxic polypeptide indicates random integration in the cellular genome. In some embodiments, the recombinant nucleic acid comprises any of the acceptor sites described above to generate any of the acceptor cells described above.

In some embodiments, the acceptor site further comprises nucleic acid encoding a gene expression repressor polypeptide. In some embodiments, the acceptor site comprises nucleic acid encoding a tetracycline repressor polypeptide operably linked to a promoter. In some embodiments, the constitutive promoter is a CMV promoter, a TK promoter, an eF1-alpha, an UbC promoter, a PGK promoter, a CAG promoter, an SV40 promoter, or a human β-actin promoter. In some embodiments, the promoter is a human β-actin promoter or a CAG promoter.

The acceptor cell is generated by engineering the genome of a cell to include an acceptor construct. There are several techniques known in the art, which can be used to engineer a cell into harboring an exogenous nucleic acid sequence. For example, the acceptor cell may be generated by inserting the acceptor construct into a cell via a viral transfection system. In some embodiments the retrovirus used is a lentivirus or an adenovirus. In some embodiments the acceptor construct may be a vector. The vector may be a viral vector. In some embodiments the vector is a viral vector, such as a lentiviral vector, a baculoviral vector, an adenoviral vector, or an adeno-associated viral (AAV) vectors. In some embodiments an AAV transfection system is used to deliver the acceptor construct into the acceptor cells. The AAV used can be modified and optimized depending on the cell type or locus used. For example AAV1, AAV2, AAV5 or any combination thereof can be used.

The acceptor construct may be delivered by other methods known in the art Many means of delivery are known (such as yeast systems, microvesicles, gene guns/means of attaching vectors to gold nanoparticles). In some embodiments the acceptor construct may be delivered via liposomes, nanoparticles, exosomes, microvesicles, or a gene-gun.

In some embodiments the acceptor construct is inserted into the genome of a cell by use of an RNA guided endonuclease system. In some embodiments a CRISPR system is used. In some embodiments the acceptor construct is inserted into the genome of the cell using RNA guided genome engineering via Cas9. However, any nuclease that works in an RNA guided genome engineering system works. Nucleases that can be used include Cas3, Cas8a, Cas5, Cas8b, Cas8C, Cas10d, Cse, Cse2, Csy1, Csy2, Csy3, GSU0054, Cas10, Csm2, Cmr5, Cas10, Cx11, Csx10, Csf1, Cas9, Cas4, Csn2, Cpf1, C2c1, C2c3, and C2c2. The type of endonuclease used may be dependent on the cell to be engineered and the target locus for insertion.

In some embodiments the acceptor construct is inserted into the genome of a cell by using a TALEN or a Zinc Finger endonuclease (ZFN).

RNA guided genome engineering via Cas9 offers improvements over TALEN and ZFN approaches for cell line engineering. Using ZFN for example has some limitations. Firstly, the ZFN technique requires synthesis of new vectors and RNA for the specific DNA binding sites in each new genomic integration locus that is to be modified. These typically require expensive optimization thus cost and complexity limits the flexibility of applying these techniques to more than one or two loci. By contrast, the RNA-guided system uses a single protein (Cas9) that requires only a short RNA molecule to program it for site-specific DNA recognition. The Cas9-RNA complex is thus easier to make than analogous ZFN targeting proteins and the system is consequently more flexible Cas9-RNA, complexes also have lower toxicity in mammalian cells than TALENs and ZFNs. In addition to Cas9, other nucleases associated with RNA guided genome editing can be used. RNA guided genome engineering is known in the art.

The nucleic acid encoding the acceptor site may be inserted into any part of the cell genome where it is possible to insert an exogenous sequence of DNA without disrupting transcription of an endogenous gene. In some embodiments, the acceptor site is targeting to the AAVS1 locus, the H11 locus, the CCR5 locus, the mouse ROSA26 locus or the human ortholog of the mouse ROSA26 locus, or the CLYBL locus. In some embodiments the construct is inserted in a location within the genome which is not epigenetically silenced. In some embodiments the acceptor construct is inserted into the AAVS1 genomic locus of the host cell. The AAVS1 genomic locus is located in the 1st intron of the protein phosphatase 1, regulatory subunit 12C (PPP1R12C) gene on human chromosome 19. This locus allows stable, long-term transgene expression in many cell types, including embryonic stem cells (Smith, J R et al., Stem Cells, 26(2) (2008)). In some embodiments the acceptor construct is inserted into the H11 genomic locus of the host cell. The Hipp11 (H11) locus, was first described by Hippenmeyer et al (Neutron, 68(4):695-709 (2010)) and in humans is located on chromosome 22q12.2, between the DRG1 and EIF4ENIF1 genes, approximately 700 bp 3′ to the 3′ UTR of human EIF4ENIF1. In some embodiments the acceptor construct is inserted into the CCR5 genomic locus of the host cell. The chemokine (C—C motif) receptor 5 (CCR5) gene is located on chromo-3 (positon 3p21.31) and encodes the major co-receptor for HIV-1. In some embodiments the acceptor construct is inserted into the Rosa26 genomic locus of the host cell. The human Rosa26 is located in chromosome 3 (position 3p25.3). In some embodiments the acceptor construct is inserted into the CLYBL genomic locus of the host cell. The CLBYL genomic locus is located in intron 2 of the Citrate Lyase Beta-Like (CLYBL) gene, on the long arm of chromosome 13. In some embodiments, a single copy of the nucleic acid encoding the acceptor site is incorporated into the acceptor cell genome (e.g., on a single allele of the acceptor cell genome).

The nucleic acid encoding the acceptor site may comprise two nucleic acid sequences which allow for homologous recombination into the genome of a cell. In some embodiments, where the acceptor construct is integrated into the AAVS1 genome locus of a cell, the acceptor construct comprises two AAVS1 sequences which allow for direct integration of the acceptor construct into the cell. The reporter construct may comprise one or more sequences which allow the construct to be integrated in a different genomic locus of a cell. The acceptor construct may be inserted into a locus within the genome of a cell via homologous recombination or any other way of genomic engineering known in the art. In some embodiments, the acceptor construct comprises two AAVS1 sequences which allow the acceptor construct to be directly integrated into the AAVS1 locus of a cell. In some embodiments, the acceptor construct comprises two CCR5 sequences which allow the acceptor construct to be directly integrated into the CCR5 locus of a cell. In some embodiments, the acceptor construct comprises two ROSA26 sequences which allow the acceptor construct to be directly integrated into the ROSA26 locus of a mouse cell. In some embodiments, the acceptor construct comprises two human orthologs of the mouse ROSA26 sequences which allow the acceptor construct to be directly integrated into the human ortholog of the mouse ROSA26 locus of a human cell. In some embodiments, the acceptor construct comprises two H11 sequences which allow the acceptor construct to be directly integrated into the H11 locus of a cell. In some embodiments, the acceptor construct comprises two CLYBL sequences which allow the acceptor construct to be directly integrated into the CLYBL locus of a cell.

In some embodiments of the inventions, the acceptor cell comprises a first recombinant nucleic acid for receiving a first multicistronic reporter vector and a second recombinant nucleic acid for receiving a second expression constrict, wherein the first recombinant nucleic acid is integrated into a first specific site in a host cell genomic and the second recombinant nucleic acid is integrated into a second specific site in a host cell genome. In some embodiments, the second recombinant nucleic acid encodes a polypeptide, a reporter polypeptide, a cytotoxic polypeptide, a selectable polypeptide, a constitutive Cas expression vector, an inducible Cas expression vector, a constitutive Cas9 expression vector or inducible Cas9 expression vector. In some embodiments, a reporter cell prepared from the acceptor cell, wherein a multicistronic reporter vector is integrated into the first specific site and a constitutive or inducible Cas expression vector (e.g., Cas9 expression vector) is integrated into a second specific site. In some embodiments, the invention provides a method wherein a reporter cell as described herein is arrayed in a multiwell plate and used as the basis for a screen using single or oligo pool sgRNAs.

### Multicistronic Reporter Vectors

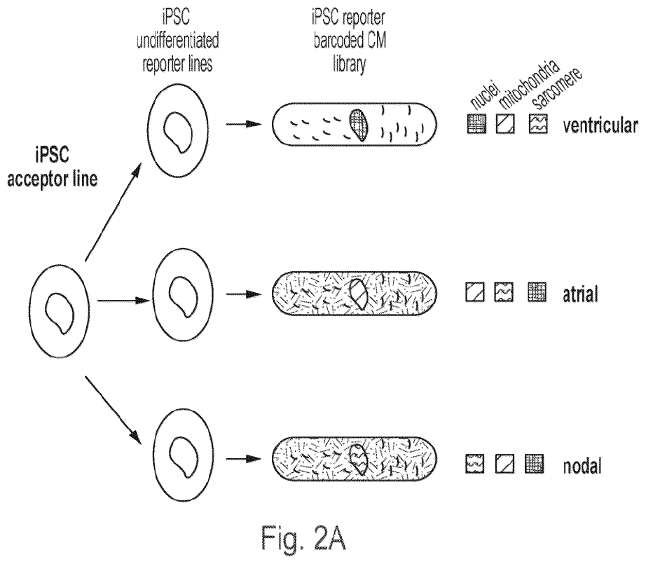

In some aspects, the invention provides multicistronic reporter vectors comprising: a promoter operably linked to an open reading frame, wherein the open reading frame comprises two or more cistrons, and wherein expression of the open reading frame in a cell yields separate component polypeptide products from each cistron; wherein each cistron comprises a multiple cloning site (MCS) and nucleic acid encoding a reporter vector, wherein each cistron encodes a different reporter polypeptide; and wherein expression of two or more nucleic acids encoding polypeptides inserted into the two or more multiple cloning sites and fused to the reporter polypeptides is essentially 1:1 stoichiometric, and wherein the vector includes at least one reporter polypeptide operably linked to a lineage-specific promoter to serve as a barcode to identify the lineage of the cell in which the multicistronic reporter vector is introduced. The vector is designed fora “plug-and-play” mode wherein different lineage specific promoters may be swapped in to drive expression of the open reading frame, different polypeptides of interest can be swapped in different reporter polypeptide can be swapped in, and different selection polypeptides may be swapped in depending on the particular use of the multicistronic reporter vector. Likewise, the multicistronic reporter vector is designed, through the use of the various MCS sequence to insert nucleic acid encoding any polypeptide of interest, such that the transgene product, tagged to a reporter polypeptide, is expressed by the multicistronic reporter vector. In some embodiments, the multicistronic vector comprises the “backbone” vector wherein transgenes of interest have not been inserted into the MCS sequences. In other embodiments, the multicistronic vector includes vectors where transgenes of interest have been inserted into the MCS sequences such that expression of the open reading yields distinct reporter-tagged polypeptides. Nonlimiting examples of multicistronic reporter vectors is provided in FIG. 2B.

In some aspects, the invention provides a multicistronic reporter vector comprising: a promoter operably linked to an open reading frame, wherein the promoter is a lineage-specific promoter, wherein the open reading frame comprises two or more cistrons, and wherein expression of the open reading frame in a cell yields separate component polypeptide products from each cistron; wherein each cistron comprises a multiple cloning site (MCS) and nucleic acid encoding a reporter vector, wherein each cistron encodes a different reporter polypeptide; and wherein expression of two or more nucleic acids encoding, polypeptides inserted into the two or more multiple cloning situs and fused to the reporter polypeptides is essentially at about 1:1 stoichiometry.

In some aspects, the invention provides a multicistronic reporter vector comprising a first promoter baked to a transactivator polypeptide, wherein the first promoter is a lineage-specific promoter; a second promoter operably linked to an open reading frame, wherein the second promoter is inducible by the transactivator polypeptide, wherein the open reading frame comprises two or more cistrons, and wherein expression of the open reading frame in a cell yields separate component polypeptide products from each cistron; wherein each cistron comprises a multiple cloning site (MCS) and nucleic acid encoding a reporter vector, wherein each cistron encodes a different reporter polypeptide; and wherein expression of two or more nucleic acids encoding polypeptides inserted into the two or more multiple cloning sites and fused to the reporter polypeptides is essentially are about 1:1 stoichiometry. In some embodiments, the transactivator polypeptide is a tetracycline transactivator polypeptide and the second promoter comprises a tetracycline responsive element. In some embodiments, the tetracycline responsive element is a Tet operator 2 (TetO2) inducible or repressor element.

In some aspects, the invention provides a multicistronic reporter vector comprising: a first promoter linked to a nucleic acid encoding a nuclear polypeptide, wherein the first promoter is a lineage-specific promoter; a second promoter operably linked to an open reading fame, wherein the second promoter is a constitutive promoter, wherein the open reading frame comprises two or more cistrons, and wherein expression of the open reading frame in a cell yields separate component polypeptide products from each cistron; wherein each cistron comprises a multiple cloning site (MCS) and nucleic acid encoding a reporter vector, wherein each cistron encodes a different reporter polypeptide, and wherein expression of two or more nucleic acids encoding polypeptides inserted into the two or more multiple cloning sites and fused to the reporter polypeptides is essentially at about 1:1 stoichiometry. In some embodiments, the housekeeping polypeptide is H2B.

In some embodiments, the cistrons of the multicistronic reporter vector are separated from one another by nucleic acid encoding one or more self-cleaving peptide and/or one or more internal ribosome, entry site (RES). In some embodiments, the one or mon self-cleaving peptides is a viral self-cleaving peptide. In some embodiments, the one or more viral self-cleaving peptides is one or more 2A peptides. In some embodiments, the one or more 2A peptides is a T2A peptide, a P2A peptide, an E2A peptide or a F2A peptide. In some embodiments, one or more of the cistrons of the open reading frame is separated from the other cistrons in the open reading frame by an IRES sequence. In some embodiments, the IRES is an encephalomyocarditis virus (EMCV) IRES, a Hepatitis C virus (HCV) IRES or an Enterovirus 71 (EV71) IRES.

In some embodiments, the multicistronic reporter vector comprises two cistrons wherein the two cistrons are separated by nucleic acid encoding a viral s-elf-cleaving peptide or an IRES. In some embodiments, the multicistronic reporter vector comprises three cistrons wherein the three cistrons are separated by nucleic acid encoding viral self-cleaving peptides. In some embodiments, the multicistronic reporter vector comprises three cistrons wherein the three cistrons are separated by IRES sequences. In some embodiments, the multicistronic reporter vector comprises three cistrons wherein the first and second cistrons are separated by nucleic acid encoding a viral self-cleaving peptide and the second and third cistron is separated by an IRES sequence. In some embodiments, the multicistronic reporter vector comprises three cistrons wherein the first and second cistrons are separated by an IRES sequence and the second and third cistron is separated by nucleic acid encoding a self-cleaving peptide. In some embodiments, the multicistronic reporter vector comprises four cistrons wherein the cistrons are separated from one another by nucleic acid encoding viral self-cleaving peptides. In some embodiments, the multicistronic reporter vector comprises four cistrons wherein the cistrons are separated from one another by IRES sequences. In some embodiments, the multicistronic reporter vector comprises four cistrons wherein the first and second cistrons are separated by nucleic acid encoding viral self-cleaving peptides, the second and third cistrons are separated by nucleic acid encoding a viral self-cleaving peptide, and the third and fourth cistrons are separated by an IRES sequence. In some embodiments, the multicistronic reporter vector comprises four cistrons wherein the first and second cistrons are separated by nucleic acid encoding viral self-cleaving peptides, the second and third cistrons are separated by an IRES sequence, and the third and fourth cistrons are separated by nucleic acid encoding a viral self-cleaving peptide. In some embodiments, the multicistronic reporter vector comprises four cistrons wherein the first and second cistrons are separated by an IRES sequence, the second and third cistrons are separated by nucleic acid encoding viral self-cleaving peptides, and the third and fourth cistrons are separated by nucleic acid encoding a viral self-cleaving peptide. In some embodiments, the multicistronic reporter vector comprises four cistrons wherein the first and second cistrons are separated by an IRES sequence, the second and third cistrons are separated by an IRES sequence, and the third and fourth cistrons are separated by nucleic acid encoding a viral self-cleaving peptide. In some embodiments, the multicistronic reporter vector comprises four cistrons wherein the first and second cistrons are separated by nucleic acid encoding viral self-cleaving peptides, the second and third cistrons are separated by an IRES sequence, and the third and fourth cistrons are separated by an IRES sequence. In some embodiments, the multicistronic reporter vector comprises four cistrons wherein the first and second cistrons are separated by an IRES sequence, the second and third cistrons are separated by nucleic acid encoding viral self-cleaving peptides, and the third and fourth cistrons are separated by an IRES sequence. In some embodiments, the multicistronic reporter vectors comprises five or more cistrons wherein the cistrons am separated from each other by any combination of nucleic acid encoding viral self-cleaving peptides and IRES sequences.

In some embodiments, the multicistronic reporter vector of the inventions comprises one or more nucleic acids encoding a peptide liker between one or more of the reporter polypeptides and one or more of the self-cleaving peptides. In some embodiments, the peptide linker comprises the sequence Gly-Ser-Gly.

In some embodiments, the invention provides multicistronic reporter vectors comprising an open reading frame is operably linked to a promoter and wherein the open reading frame includes two or more MCS sequences linked to nucleic acid encoding a reporter polypeptide such that when nucleic acid encoding a transgene of interest is inserted into an MCS, the resulting polypeptide encoded by the multicistronic reporter vector includes the product of the transgene of interested tagged with the reporter polypeptide. Each cistron of the open reading frame encodes a deferent reported polypeptide such that each tagged transgene product may be profiled in a live cell. In some embodiments, the reporter polypeptide is a fluorescent reporter polypeptide. In some embodiments, the reporter polypeptide may be a green fluorescent protein (GFP) or any of its derivatives. In some embodiments the reporter polypeptide is a non GFP derived fluorescent peptide. In some embodiments the reporter polypeptide is GFP, EGFP, Emerald, Citrine, Venus, mOrange, mCherry, TagBFP, mTurquoise, Cerulean, UnaG, dsRed, eqFP611, Dronpa, RFP, TagRFPs, TdTomato, KFP, EosFP, Dendra, IrisFP, iRFPs, or smURFP. In some embodiments, the reporter polypeptide is a luciferase. In some embodiments, the reporter polypeptide is an enzyme, which when expressed allows for visualization of expression through the products of a chemical reaction. In some embodiments the reporter domain is a firefly luciferase or a Renilla luciferase. In some embodiments the reporter domain is β-glucuronidase or β-galactosidase.

In some embodiments, the invention provides a multicistronic reporter vector, wherein the vector comprises a promoter operably linked to an open reading frame, wherein the open reading frame comprises a first cistron and a second cistron, wherein each cistron comprises 5′ to 3′ nucleic acid comprising a MCS, nucleic acid encoding a reporter polypeptide, and nucleic acid encoding a linker peptide; wherein the first cistron and the second cistron are separated by nucleic acid encoding a viral cleavage peptide. In some embodiments, the invention provides a multicistronic reporter vector, wherein the vector comprises a promoter operably linked to an open reading frame, wherein the open reading frame comprises a first cistron, a second cistron and a third cistron, wherein each cistron comprises 5′ to 3′ nucleic acid comprising a MCS, nucleic acid encoding a reporter polypeptide, and nucleic acid encoding a linker peptide; wherein the first cistron and the second cistron are separated by nucleic acid encoding a first viral cleavage peptide and the second cistron and the third cistron ae separated by nucleic acid encoding a second viral cleavage peptide. In some embodiments, the invention provides a multicistronic reporter vector, wherein the vector comprises a promoter operably linked to an open reading frame, wherein the open reading frame comprises a first cistron, a second cistron, a third cistron and a fourth cistron, wherein each cistron comprises 5′ to 3 nucleic acid comprising a MCS, nucleic acid encoding a reporter polypeptide nucleic acid encoding a linker peptide; wherein the first cistron and the second cistron are separated by nucleic acid encoding a first viral cleavage peptide, the second cistron and the third cistron are separated by nucleic acid encoding a second viral cleavage peptide the third cistron and the fourth cistron are separated by nucleic acid encoding a third viral cleavage peptide. In some embodiments, the invention provides a multicistronic reporter vector, wherein the vector comprises a promoter operably linked to an open reading frame, wherein the open reading frame comprises a first cistron, a second cistron, a third cistron and a fourth cistron, wherein each cistron comprises 5′ to 3′ nucleic acid comprising a MCS, nucleic acid encoding a reporter polypeptide, nucleic acid encoding a linker peptide; wherein the first cistron and the second cistron are separated by nucleic acid encoding a first viral cleavage peptide, the second cistron and the third cistron are separated by nucleic acid encoding a second viral cleavage peptide the third cistron and the fourth cistron are separated by nucleic acid encoding an IRES.

In some embodiments, the multicistronic reporter vector further comprises one or more inducible elements located between the promoter and open reading frame. In some embodiments, the multicistronic reporter vector comprises two inducible elements. In some embodiments, the inducible element is a Tet operator 2 (TetO2) inducible element.

In some embodiments, the invention provides multicistronic reporter vectors wherein the vector comprises an open reading frame comprising two or more cistrons, wherein the open reading frame is operably linked to an inducible promoter. In some embodiments, the inducible promoter is a tetracycline responsive promoter. In some embodiments, the inducible promoter is a rapamycin-regulated promoter or a sterol inducible promoter.

In some embodiments, the invention provides multicistronic reporter vectors wherein the vector comprises an open reading frame comprising two or more cistrons, wherein the open reading frame is operably linked to a lineage specific promoter or a sublineage specific promoter. In some embodiments, the lineage specific promoter is an endoderm specific promoter, a mesoderm specific promoter, or an ectoderm specific promoter. Examples of cardiomyocyte specific promoters are: myosin light chain 2v (MLC2v) promoter, sarcolipin (SLN) promoter, short stature homeobox 2 (SHOX2) promoter: examples of neuro-specific promoters are vesicular GABA transporter (vGAT) promoter, tyrosine hydroxylase (TH) promoter, glial fibrillary acidic protein (GFAP) promoter, vesicular glutamate transporter 1 (vGLUT) promotor.

In some embodiments, the multicistronic reporter is driven by the promoter which is active only in pluripotent cells. For example the promoter operably linked to the open reading frame comprising two or more cistrons, can be Oct-4, Sox2, Nanog, KLF4, TRA-1-60, TRA-2-54, TRA-1-81, SSEA1, SSEA4 or the promoter of any pluripotency associated gene.

In some embodiments the multicistronic reporter is driven by a promoter which is specific to a stage of differentiation. For example, using an OCT-4 promoter-would allow expression of the fusion proteins encoded by the multicistronic construct to only be expressed in pluripotent cells. Using an MSP1+promoter would allow expression of the fusion proteins encoded by the multicistronic construct to only be expressed in pre-cardiac progenitor cells. Using an alpha-MHC promoter would allow expression of the fusion proteins encoded by the multicistronic construct to be expressed only in cardiomyocytes.

In some embodiments, the multicistronic reporter vector comprises nucleic acid encoding a polypeptide (e.g., a “housekeeping” polypeptide) fused to a reporter polypeptide and operably linked to a tissue specific, lineage specific or sublineage specific promoter. In some embodiments, the nucleic acid encoding the polypeptide is encoded as part of the multicistronic open reading frame of the vector. Here, the tissue-specific, lineage-specific or sublineage specific promoter drives expression of the multicistronic open reading frame. In other embodiments, the tissue-specific, lineage-specific or sublineage specific promoter and reporter polypeptide art present on the multicistronic vector as a separate transcription unit from the multicistronic open reading frame. In some embodiments, an insulator region is present between the separate transception it and the multicistronic open reading frame. In some embodiments, the separate transcription unit is 5′ to the multicistronic open reading frame. In some embodiments, the separate transcription unit is 3′ to the multicistronic open reading frame.

In some embodiments, the vector comprises nucleic acid encoding H2B fused to a reporter polypeptide and operably linked to an MLC2v promoter, an SLN promoter, or a SHOX2 promoter, thereby enabling expression of a reporter in a cardio subtype cell. In some embodiments, the invention provides multireporter cells comprising multicistronic reporter vectors as described above, wherein the vector comprises nucleic acid encoding H2B fused to a reporter polypeptide and operably linked to a vGAT promoter, a TH promoter, a GFAP promoter, or a vGLUT promoter, thereby enabling expression of a reporter in a neural subtype cell.

In some embodiments the multicistronic reporter is driven by a lineage specific promoter. In some embodiments the lineage specific promoter is a promoter active in early endodermal, early mesodermal, early ectodermal, chorionic, or trophectoderm lineage. In some embodiments the lineage specific promoter is a promoter only active in progenitor cells. In some embodiments the lineage specific promoter is a promoter only active in a hematopoietic cell, an endothelial progenitor cell, a mesenchymal progenitor cell, a neural progenitor cell, an osteochondral progenitor cell, a lymphoid progenitor cell or a pancreatic progenitor cell. In some embodiments the multireporter cell is a cord endothelial cell, a cord blood stem cell, an adipose-derived stem cell, a hepatocyte, a keratinocyte, a neural stem cell, a pancreatic beta-cell, a lymphocyte progenitor cell or an amniotic cell.

In some embodiments, the lineage specific promoter is a promoter of a cell derived from extraembryonic tissue, ectoderm, endoderm or mesoderm. In some embodiments the lineage specific promoter is a promoter that is expressed in cardiomyocytes, endothelial cells, neuronal cells, GABAergic neurons, astrocytes, dopaminergic neurons, glutamatergic neurons, hepatocytes, hepatoblasts, skeletal myoblasts, macrophages, cortical neurons, atrial cardiomyocytes, ventricular cardiomyocytes, nodal cardiomyocytes, Purkinje fibers, basal cells, squamous cells, renal cells, pancreatic beta cells, epithelial cells, mesenchymal cells, adrenocortical cells, osteoblasts, osteocytes, chondroblasts, chondrocytes, gastrointestinal cells, colorectal cells, ductal cells, lobular cells, lymphocytes, retinal cells, photoreceptor cells or cochlear cells.

In some embodiments, the multicistronic reporter is driven by the promoter which is active only in pluripotent cells. For example the promoter operably linked to the open reading frame comprising two or more cistrons, can be Oct-4, Sox2, Nanog, KLF4, TRA-1-60, TRA-2-54, TRA-1-81, SSEA1, SSEA4 or the promoter of any pluripotency associated gene.

In some embodiments the multicistronic reporter is driven by a promoter which is specific to a stage of differentiation. For example, using an OCT-4 promoter would allow expression or the fusion proteins encoded by the multicistronic construct to only be expressed in pluripotent cells Using an MSP1+promoter would allow expression of the fusion proteins encoded by the multicistronic construct to only be expressed in pre-cardiac progenitor cells. Using an alpha-MHC promoter would allow expression of the fusion proteins encoded by the multicistronic construct to be expressed only in cardiomyocytes.

In some embodiments, the multicistronic reporter vector comprises nucleic acid encoding a polypeptide (e.g., a “housekeeping” polypeptide) fused to a reporter polypeptide and operably linked to a tissue specific, lineage specific or sublineage specific promotor In some embodiments, the nucleic acid encoding the polypeptide is encoded as part of the multicistronic open reading frame of the vector Here, the tissue-specific, lineage-specific or sublineage specific promoter drives expression of the multicistronic open reading frame. In other embodiments, the tissue-specific, lineage-specific or sublineage specific promoter and reporter polypeptide are present on the multicistronic vector as a separate transcription unit from the multicistronic open reading frame. In some embodiments, an insulator region is present between the separate transcription unit and the multicistronic open reading frame. In some embodiments, the separate transcription unit is 5′ to the multicistronic open reading frame. In some embodiments, the separate transcription unit is 3′ to the multicistronic open reading frame.

In some embodiments, the vector comprises nucleic acid encoding H2B fused to a reporter polypeptide and operably linked to an MLC2v promoter, an SLN promoter, or a SHOX2 promoter, thereby enabling expression of a reporter in a cardo subtype cell. In some embodiments, the invention provides multireporter cells comprising multicistronic reporter vectors as described above, wherein the vector comprises nucleic acid encoding H2B fused to a reporter polypeptide and operably linked to a vGAT promote, a T1 promoter, a GFAP promoter, or a vGLUT promoter, thereby enabling expression of a reporter in a neural subtype cell.

In some embodiments the multicistronic reporter is driven by a lineage specific promoter. In some embodiments the lineage specific promoter is a promoter active in early endodermal, early mesodermal, early ectodermal, chorionic, or trophectoderm lineage. In some embodiments the lineage specific promoter is a promoter only active in progenitor cells. In some embodiments the lineage specific promoter is a promotor only active in a hematopoietic cell, an endothelial progenitor cell, a mesenchymal progenitor cell, a neural progenitor cell, an osteochondral progenitor cell, a lymphoid progenitor cell or a pancreatic progenitor cell. In some embodiments the multireporter cell is a cord endothelial cell, a cord blood stem cell, an adipose-derived stem cell, a hepatocycte, a keratinocyte, a neural stem cell, a pancreatic beta-cell, a lymphocyte progenitor cell or an amniotic cell.

In some embodiments, the lineage specific promoter is a promoter of a cell derived from extraembryonic tissue, ectoderm, endoderm or mesoderm. In some embodiments the lineage specific promoter is a promoter that is expressed in cardiomyocytes, endothelial cells, neuronal cells, GABAergic neurons, astrocytes, dopaminergic neurons, glutamatergic neurons, hepatocytes, hepatoblasts, skeletal myoblasts, macrophages, cortical neurons, atrial cardiomyocytes, ventricular cardiomyocytes, nodal cardiomyocytes, Purkinje fibers, basal cells, squamous cells, renal cells, pancreatic beta cells, epithelial cells, mesenchymal cells, adrenocortical cells, osteoblasts, osteocytes, chondroblasts, chondrocytes, gastrointestinal cells, colorectal cells, ductal culls, lobular cells, lymphocytes, retinal cells, photoreceptor cells or cochlear cells.

In some embodiments, the invention provides multicistronic reporter vectors wherein the vector comprises an open reading frame comprising two or more cistrons, wherein the open reading frame is operably linked to a tissue specific promoter. In some embodiments, the tissue specific promoter is specific for cells of heart, blood, muscle, lung, liver, kidney, pancreas, brain, skin, or other tissue-specific lineage.

In some embodiments, the invention provides multicistronic reporter vectors wherein the vector comprises an open reading frame comprising two or more cistrons, wherein the vector further comprises a site-specific recombinase sequence located 3′ to the open reading frame. Examples of site-specific recombinase sequences include but are not limited to a FRT nucleic acid sequence, an attP nucleic acid sequence and loxP nucleic acid sequence. In some embodiments, the site-specific recombinase sequence is a FRT nucleic acid. In some embodiments, the site-specific recombinase sequence is an attP nucleic acid. In some embodiments, the site-specific recombinase sequence is an attB nucleic acid. In some embodiments, the site-specific recombinase sequence is a loxP nucleic acid. In some embodiments, the site-specific recombinase sequence is a FRT nucleic acid and an attP sequence. In some embodiments, the site-specific recombinase sequence is a FRT nucleic acid and an attB sequence.

In some embodiments, the invention provides multicistronic reporter vectors wherein the vector comprises an open reading frame comprising two or more cistrons, wherein the vector further comprises nucleic acid encoding a selectable marker, wherein the nucleic acid encoding the selectable marker is not operably linked to the promoter when the site-specific recombinase sequence has not recombined and is operably linked to the promotor when the site-specific recombinase sequence recombines with its target site-specific recombinase sequence. In some embodiments, the selectable marker confers resistance to hygromyocin, Zeocin™, puromycin, neomycin or an analog of hygromyocin, Zeocin™, puromycin, blasticidin or neomycin.

In some embodiments, the invention provides multicistronic reporter vectors wherein the vector comprises an open reading frame comprising two or more cistrons, wherein nucleic acid encoding one or more polypeptides is inserted in-frame into the one or more MCS. In some embodiments, the one or more polypeptides comprise polypeptides that can be used to profile or distinguish a single or multiple biological pathways, cross-talk between two or more biological pathways, synthetic lethality, cellular homeostasis, organelle homeostasis, cell-cell interactions, a toxicity response or other cellular or subcellular phenotypes. In some embodiments, the one or more polypeptides comprise polypeptides that can be used to profile phenotypic features of a cell. In some embodiments, the one or more polypeptides include ATF4, ATF6, XBPL1ΔDBBD and H2B; alpha-tubulin, mitochondrial targeting sequence (MTS), LC3 and H2B; or 53BP1, Nrf2, p53RE and H2B; Mek, Erk, Raf and Ras; H2B, palmitoylation signal and MTS; or H12B, MTS and alpha-actinin2. In some embodiments, the invention provides multiple multicistronic reporter vectors, wherein the multiple multicistronic reporter vectors are used to profile a specific target selected from a single biological pathway, cross-talk between two or more biological pathways, cellular homeostasis, organelle homeostasis and a toxicity response; wherein each vector encodes at least one common polypeptide (e.g., H2B) that can be used to identify cells that received one or more of the multicistronic vectors encoding polypeptides targeted to a specific target. In some respects, the common polypeptide may be considered a barcode for the specific target.

In some embodiments, the multicistronic reporter vector of the invention includes at least one cistron comprising nucleic acid encoding an organelle marker. In some embodiments, the organelle marker comprises H2B, α-actinin 2 or a mitochondrial targeting signal fused to the reporter polypeptide.

In some embodiments, the invention provides a multicistronic reporter vector as described above, wherein the vector comprises one, two or three transcription units comprising a promoter and nucleic acid encoding a transgene located 5′ to the open reading frame comprising two or more cistrons, wherein the reporter vector further comprises a core insulator sequence and a polyA sequence located 3′ to the transcription units and 5′ to the open reading frame comprising two or more cistrons. In some embodiments, the one, two, or three transcription units encode transcription factors, or other factors that may aid in the assays described herein.

### Multireporter Cells

In some embodiments, the invention provides a multireporter stem cell comprising any of the acceptor cells described above in which a multicistronic reporter vector described above, has integrated into the genome of the acceptor cell, wherein the cell includes at least one reporter polypeptide operably linked to a lineage-specific promotor to serve as a barcode to identify the lineage of the cell. In some embodiments, the multicistronic reporter vector has integrated into a specific site in an acceptor cell genome. In some embodiments, the specific site in the acceptor cell genome is an adeno-associated virus S1 (AAVS1) locus, a chemokine (CC motif) receptor 5 (CCR5) locus, a human ortholog of the mouse ROSA26 locus, or the citrate lyase beta like gene locus (CLYBL). In some embodiments, a single copy of the multicistronic reporter vector is integrated into the acceptor cell genome.

In some embodiments, any of the multicistronic reporter vectors described above is inserted into an acceptor cell to generate a multireporter cell of the invention.

In some aspects, the Invention provides a multireporter stent cull comprising a multicistronic reporter vector comprising: a promoter operably linked to an open reading frame, wherein the promoter is a lineage-specific promoter, wherein the open reading frame comprises two or more cistrons, and wherein expression of the open reading frame in a cell yields separate component polypeptide products from each cistron: wherein each cistron comprises a multiple cloning site (MCS) and nucleic acid encoding a reporter-vector, wherein each cistron encodes a different reporter polypeptide; and wherein expression of two or more nucleic acids encoding polypeptides inserted into the Iwo or more multiple cloning sites and fused to the reporter polypeptides is essentially at about 1:1 stoichiometry.

In some aspects, the intention provides a multireporter stem cell comprising a multicistronic reporter vector comprising: a first promoter linked to a transactivator polypeptide, wherein the first promoter is a lineage-specific promoter, a second promoter operably linked to an open reading frame, wherein the second promoter is inducible by the transactivator polypeptide, wherein the open reading frame comprises two or more cistrons, and wherein expression of the open reading frame in a cell yields separate component polypeptide products from each cistron; wherein each cistron comprises a multiple cloning site (MCS) and nucleic acid encoding a reporter vector, wherein each cistron encodes a different reporter polypeptide; and wherein expression of two or more nucleic acids encoding polypeptides inserted into the two or more multiple cloning sites and fused to the reporter polypeptides is essentially at about 1:1 stoichiometry. In some embodiments, the transactivator polypeptide is a tetracycline transactivator polypeptide and the second promoter comprises a tetracycline responsive element. In some embodiments, the tetracycline responsive element is a Tet operator 2 (TetO2) inducible element. In some embodiments, the tetracycline responsive element is a Tet operator 2 (TetO2) repressor element.

In some aspects, the invention provides a multireporter stem cell comprising a multicistronic reporter vector comprising a first promoter linked to a nucleic acid encoding a nuclear polypeptide, wherein the first promoter is a lineage-specific promoter, a second promoter operably linked to an open reading frame, wherein the second promoter is a constitutive promoter, wherein the open reading frame comprises two or more cistrons, and wherein expression of the open reading frame in a cell yields separate component polypeptide products from each cistron; wherein each cistron comprises a multiple cloning site (MCS) amu nucleic acid encoding a reporter vector, wherein each cistron encodes a different reporter polypeptide, and wherein expression of two or more nucleic acids encoding polypeptides inserted into the two or more multiple cloning sites and fused to the reporter polypeptides is essentially at about 1:1 stoichiometry. In some embodiments, the housekeeping polypeptide is H2B.

In some embodiments, the invention provides a multireporter stem cell, wherein the reporter cell comprises a multicistronic reporter construct, wherein the multicistronic reporter construct comprises a lineage specific promoter operably linked to an open reading frame, wherein the open reading frame comprises two or more cistrons; wherein each cistron comprises a nucleic acid encoding a different transgene product fused to a different reporter polypeptide, wherein expression of the open reading frame in a cell yields separate component polypeptide products from each cistron, and wherein expression of the transgene products is essentially 1:1 stoichiometric. In some embodiments, the cistrons are separated from one another by nucleic acid encoding one or more self-cleaving peptide and/or one or more internal ribosome entry site (IRES).

In some embodiments, the cistrons of the multicistronic reporter vector inserted in the multireporter stem cell are separated from one another by nucleic acid encoding one or more self-cleaving peptide and/or one or more internal ribosome entry site (IRES). In some embodiments, the one or more self-cleaving peptides is a viral self-cleaving peptide. In some embodiments, the one or more viral self-cleaving peptides is one or more 2A peptides. In some embodiments, the one or mort 2A peptides is a T2A peptide, a P2A peptide, an E2A peptide or a F2A peptide. In some embodiments, one or more of the cistrons of the open reading frame is separated from the other cistrons in the open reading frame by an IRES sequence. In some embodiments, the IRES is an encephalomyocarditis virus (EMCV) IRES, a Hepatitis C virus (HCV) IRES or an Enterovirus 71 (EV71) IRES.

In some embodiments, the multireporter stem cell comprises a multicistronic reporter vector wherein the multicistronic reporter vector comprises two cistrons wherein the two cistrons are separated by nucleic acid encoding a viral self-cleaving peptide or an IRES. In some embodiments, the multicistronic reporter vector comprises three cistrons wherein the three cistrons are separated by nucleic acid encoding viral self-cleaving peptides. In some embodiments, the multicistronic reporter vector comprises three cistrons wherein the three cistrons are separated b IRES sequences. In some embodiments, the multicistronic reporter vector comprises three cistrons wherein the first and second cistrons are separated by nucleic acid encoding a viral self-cleaving peptide and the second and third cistron is separated by an IRES sequence. In some embodiments, the multicistronic reporter vector comprises three cistrons wherein the first and second cistrons are separated by an IRES sequence and the second and third cistron is separated by nucleic acid encoding a self-cleaving peptide. In some embodiments, the multicistronic reporter vector comprises four cistrons wherein the cistrons are separated from one another by nucleic acid encoding viral self-cleaving peptides. In some embodiments, the multicistronic reporter vector comprises; four cistrons wherein the cistrons are separated from one another by IRES sequences. In some embodiments, the multicistronic reporter vector comprises four cistrons wherein the first and second cistrons are separated by nucleic acid encoding viral self-cleaving peptides, the second and third cistrons are separated by nucleic acid encoding a viral self-cleaving peptide, and the third and fourth cistrons are separated by an IRES sequence. In some embodiments, the multicistronic reporter vector comprises four cistrons wherein the first and second cistrons are separated by nucleic acid encoding viral self-cleaving peptides, the second and third cistrons are separated by nucleic acid encoding a viral self-cleaving peptide, and the third and fourth cistrons are separated by an IRES sequence. In some embodiments, the multicistronic reporter vector comprises four cistrons wherein the first and second cistrons are separated by nucleic acid encoding viral self-cleaving peptides, the second and third cistrons are separated by an IRES sequence, and the third and fourth cistrons are separated by nucleic acid encoding a viral self-cleaving peptide. In some embodiments, the multicistronic reporter vector comprises four cistrons wherein the first and second cistrons are separated by an IRES sequence, the second and third cistrons are separated by nucleic acid encoding viral self-cleaving peptides, and the third and fourth cistrons arm separated by nucleic acid encoding a viral self-cleaving peptide. In some embodiments, the multicistronic reporter vector comprises four cistrons wherein the first and second cistrons are separated by an IRES sequence, the second and third cistrons are separated by an IRES sequence, and the third and fourth cistrons are separated by nucleic acid encoding a viral self-cleaving peptide. In some embodiments, the multicistronic reporter vector comprises four cistrons wherein the first and second cistrons are separated by nucleic acid encoding viral self-cleaving peptides, the second and third cistrons are separated by an IRES sequence, and the third and fourth cistrons are separated by an IRES sequence. In some embodiments, the multicistronic reporter vector comprises four cistrons wherein the first and second cistrons are separated by an IRES sequence, the second and third cistrons are separated by nucleic acid encoding viral self-cleaving peptides, and the third and fourth cistrons are separated by an IRES sequence. In some embodiments, the multicistronic reporter vectors comprises five or more cistrons wherein the cistrons are separated from each other by any combination of nucleic acid encoding viral self-cleaving peptides and IRES sequences.

In some embodiments, the multireporter stem cell comprises a multicistronic reporter vector which comprises one or more nucleic acids encoding a peptide linker between one or more of the reporter polypeptides and one or more of the self-cleaving peptides. In some embodiments, the peptide linker comprises the sequence Gly-Ser-Gly.

In some embodiments, the invention provides multireporter stem cells comprising a multicistronic reporter vector which comprises an open reading frame is operably linked to a promoter and wherein the open reading frame includes two or more MCS sequences linked to nucleic acid encoding a reporter polypeptide such that when nucleic acid encoding a transgene of interest is inserted into an MCS, the resulting polypeptide encoded by the multicistronic reporter vector includes the product of the transgene of interested tagged with the reporter polypeptide. Each cistron of the open reading flame encodes a different reported polypeptide such that each tagged transgene product may be profiled in a live cell. In some embodiments, the reporter polypeptide is a fluorescent reporter polypeptide. In some embodiments, the reporter polypeptide may be a green fluorescent protein (GFP) or any of its derivatives. In some embodiments the reporter polypeptide is a non GFP derived fluorescent peptide. In some embodiments the reporter polypeptide is GFP, EGFP, Emerald, Citrine, Venus, mOrange, mCherry, TagBFP, mTurquoise, Cerulean, UnaG, dsRed, eqFP611, Dronpa, RFP, TagRFPs, TdTomato, KFP, EosFP, Dendra, IrisFP, iRFPs, or smURFP. In some embodiments, the reporter polypeptide is a luciferase. In some embodiments, the reporter polypeptide is an enzyme, which when expressed allows for visualization of expression through the products of a chemical reaction. In some embodiments the reporter domain is a firefly luciferase or a Renilla luciferase. In some embodiments the reporter domain is β-glucuronidase or β-galactosidase.

In some embodiments, the invention provides a multireporter stem cell comprising a multicistronic reporter vector, wherein the vector comprises a promoter operably linked to an open reading frame, wherein the open reading frame comprises a first cistron and a second cistron, wherein each cistron compress 5′ to 3′ nucleic acid comprising a MCS, nucleic acid encoding a reporter polypeptide, and nucleic acid encoding a linker peptide; wherein the first cistron and the second cistron are separated by nucleic acid encoding a viral cleavage peptide. In some embodiments, the invention provides a multicistronic reporter vector, wherein the vector comprises a lineage specific promoter operably linked to an open reading frame, wherein the open reading frame comprises a first cistron, a second cistron and a third cistron, wherein each cistron comprises 5′ to 3 nucleic acid comprising a MCS, nucleic acid encoding a reporter polypeptide, and nucleic acid encoding a linker peptide, wherein the first cistron and the second cistron are separated by nucleic acid encoding a first viral cleavage peptide and the second cistron and the third cistron am separated by nucleic acid encoding a second viral cleavage peptide. In some embodiments, the invention provides a multicistronic reporter vector, wherein the vector comprises a promoter operably linked to an open reading frame, wherein the open reading frame comprises a first cistron, a second cistron, a third cistron and a fourth cistron, wherein each cistron comprises 5′ to 3′ nucleic acid comprising a MCS, nucleic acid encoding a reporter polypeptide nucleic acid encoding a linker peptide; wherein the first cistron and the second cistron are separated by nucleic acid encoding a first viral cleavage peptide, the second cistron and the third cistron are separated by nucleic acid encoding a second viral cleavage peptide the third cistron and the fourth cistron are separated by nucleic acid encoding a third viral cleavage peptide. In some embodiments, the invention provides a multicistronic reporter vector, wherein the vector comprises a promoter operably linked to an open reading frame, wherein the open reading frame comprises a first cistron, a second cistron, a third cistron and a fourth cistron, wherein each cistron comprises 5′ to 3′ nucleic acid comprising a MCS, nucleic acid encoding a reporter polypeptide, nucleic acid encoding a linker peptide; wherein the first cistron and the second cistron are separated by nucleic acid encoding a first viral cleavage peptide, the second cistron and the third cistron are separated by nucleic acid encoding a second viral cleavage peptide the third cistron and the fourth cistron are separated by nucleic acid encoding an IRES.

In some embodiments, the multicistronic: reporter vector of the multireporter stem cell further comprises one or more inducible elements located between the promoter and open reading frame. In some embodiments, the multicistronic reporter vector comprises two inducible elements. In some embodiments, the inducible element is a Tet operator 2 (TetO2) inducible element. In some embodiments, the responsive element is a Tet operator 2 (TetO2) repressor element.

In some embodiments, the invention provides multireporter stem cells comprising a multicistronic reporter vector wherein the vector comprises an open reading frame comprising two or more cistrons, wherein the open reading frame is operably linked to a promoter. In some embodiments, the promoter is a constitutive promoter. In some embodiments, the constitutive promoter is a Cytomegalovirus a (CMV), a Thymidine Kinase (TK), an eF1-alpha, a Ubiquitin C (UbC), a Phosphoglycerate Kinase (PGK), a CAG promoter, an SV40 promoter, or a human β-actin promoter.

In some embodiments, the invention provides multireporter stem cells comprising a multicistronic reporter vector wherein the vector comprises an open reading frame comprising two or mom cistrons, wherein the open reading frame is operably linked to an inducible promoter. In some embodiments, the inducible promoter is a tetracycline responsive promoter. In some embodiments, the inducible promoter is a rapamycin-regulated promoter or a sterol inducible promoter.

In some embodiments, the invention provides multireporter stem cells which comprise a multicistronic reporter vector wherein the vector comprises an open reading frame comprising two or more cistrons, wherein the open reading frame is operably linked to a lineage specific promoter or a sublineage specific promoter. In some embodiments, the lineage specific promoter is an endoderm specific promoter, a mesoderm specific promoter, or an ectoderm specific promoter.

In some embodiments, the invention provides a multireporter stem cell which comprises a multicistronic reporter vector wherein the vector comprises an open reading frame comprising two or more cistrons, wherein the open reading frame is operably linked to a tissue specific promoter. In some embodiments, the tissue specific promoter is specific for cells of heat, blood, muscle, lung, liver, kidney, pancreas, brain, skin, or other tissue-specific lineage.

In some embodiments, the invention provides multireporter cells comprising a multicistronic reporter vector wherein the vector comprises an open reading frame comprising two or more cistrons, wherein the vector further comprises a site-specific recombinase sequence located 3′ to the open reading frame which was used to target the multicistronic reporter vector to a specific site in the cell. Examples of site-specific recombinase sequences include but are not limited to a FRT nucleic acid sequence, an attP nucleic acid sequence and loxP nucleic acid sequence. In some embodiments, the site-specific recombinase sequence is a FRT nucleic acid. In some embodiments, the site-specific recombinase sequence is an attP nucleic acid. In some embodiments, the site-specific recombinase sequence is an attB nucleic acid. In some embodiments, the site-specific recombinase sequence is a loxP nucleic acid. In some embodiments, the site-specific recombinase sequence is a FRT nucleic acid and an attP sequence. In some embodiments, the site-specific recombinase sequence is a FRT nucleic acid and an attB sequence.

In some embodiments, the invention provides multireporter cells which comprise a multicistronic reporter vector wherein the vector comprises an open reading frame comprising two or more cistrons, wherein the vector further comprises nucleic acid encoding a selectable marker, wherein the nucleic acid encoding the selectable marker is not operably linked to the promoter when the site-specific recombinase sequence has not recombined and is operably linked to the promoter when the site-specific recombinase sequence recombines with its target site-specific recombinase sequence. In some embodiments, the selectable marker confers resistance to hygromyocin, Zeocin™, puromycin, neomycin or an analog of hygromyocin, Zeocin™, puromycin, blasticidin or neomycin.

In some embodiments, the invention provides multireporter cells comprising a multicistronic reporter vector wherein the vector comprises an open reading frame comprising two or more cistrons, wherein each cistron encodes a transgene fused to a reporter polypeptide. In some embodiments, the transgenes encode polypeptides that can be used to profile or distinguish a single or multiple biological pathways, cross-talk between two or more biological pathways, synthetic lethality, cellular homeostasis, organelle homeostasis, cell-cell interactions, a toxicity response or other cellular or subcellular phenotypes. In some embodiments, the transgenes encode polypeptides that can be used to profile phenotypic features of a cell. In some embodiments, the polypeptides include ATF4, ATF6, XBP1ΔDBBD and H2B: alpha-tubulin, mitochondrial targeting sequence (MTS), LC3 and H2B; or 53BP1, Ntf2, p53RE and H2B; Mek, Erk, Raf and Ras: H2B, palmitoylation signal and MTS; or 14213. MTS and alpha-actinin2.

In some embodiments, the invention provides a multireporter cells which comprise a multicistronic reporter vector as described above, wherein the vector comprises one, two or three additional transcription units comprising a promoter and nucleic acid encoding a transgene located 5′ to the open reading frame comprising two or more cistrons, wherein the reporter vector further comprises a core insulator sequence and a polyA sequence located 3′ to the transcription units and 5′ to the open reading frame comprising two or more cistrons. In some embodiments, the one, two, or three transcription units encode transcription factors, or other factors that may aid in the assays described herein. In some embodiments, at least of the additional transcription units comprises nucleic acid encoding a reporter molecule operably linked to a promoter. In some embodiments, the reporter molecule is operably linked to a lineage specific promoter. In some embodiments, the multireporter cell comprising the additional transcription unit encoding the reporter molecule operably linked to a lineage specific promoter is a pluripotent stem cell (e.g., an iPSC).

In some embodiments, the multireporter cell nay be a stem cell which can be differentiated into different lineages. For example the multireporter cell can be a totipotent, pluripotent, multipotent or progenitor stem cell. In some embodiments the multireporter cell is a totipotent stem cell which has the ability to at least differentiate into all embryonic and extraembryonic lineages. In some embodiments the multireporter cell is a pluripotent stem cell. In some embodiments the reporter pluripotent stem cell is an embryonic pluripotent stem cell isolated from an animal. In a particular embodiment the reporter pluripotent stem cell is a mammalian embryonic stem cell. In some embodiments the reporter pluripotent stem cell is a human embryonic stem cell. In some embodiments the reporter pluripotent stem cell is an induced pluripotent stem cell. The iPS cell used to develop the reporter iPS cell may have been generated by reprogramming via transfection, piggy-Bac, episomal, or protein reprogramming methods. The iPS used to develop the reporter iPS cell may have been generated by reprogramming a somatic, terminally differentiated or partially differentiated cell of an ectodermal, endodermal, mesodermal, placental, chorionic, or trophectodermal lineage. For example the reporter iPS cell may have been derived from a fibroblast, a peripheral blood cell, a cord blood endothelial cell, a cord blood stem cell, an adipose-derived stem cell, a hepatocyte, a keratinocyte, a neural stem cell, a pancreatic beta-cell or an amniotic cell. The reporter iPS cell may have been derived from an established iPS cell bine, or from a patient specific iPS cell. In some embodiments the reporter iPS cell was derived from an iPS cell generated by reprogramming an immune privileged cell.

In some embodiments the multireporter cell is a multipotent or a progenitor cell. The multireporter cell may be a be a hematopoietic cell, as endothelial progenitor cell, a mesenchymal progenitor cell, a neural progenitor cell, an osteochondral progenitor cell, a lymphoid progenitor cell or a pancreatic progenitor cell. In some embodiments the multireporter cell is a cord endothelial cell, a cord blood stem cell, an adipose-derived stem cell, a hepatocyte, a keratinocyte, a neural stem cell, a pancreatic beta-cell or an amniotic cell.

The stem cell may be differentiated into any progenitor or terminal cell lineage. Methods of general or lineage specific differentiation am known in the art. The stem cell may be differentiated using any methods known in the art. For example the stem cell may be differentiated using one or more factors or molecules that drive differentiation, one or more cellular matrixes, embryoid body formation, or a combination of the above. In some embodiments, the differentiated cell is a multireporter cell.

The cells may be differentiated into cardiomyocytes, endothelial cells, neuronal cells, GABAergic neurons, astrocytes, dopaminergic neurons glutamatergic neurons, hepatocytes, hepatoblasts, skeletal myoblasts, macrophages, cortical neurons, atrial cardiomyocytes, ventricular cardiomyocytes, Purkinje fibers, basal cells, squamous cells, renal cells, pancreatic beta cells, epithelial cells, mesenchymal cells, adrenocortical cells, osteoblasts, osteocytes, chondroblasts, chondrocytes, gastrointestinal cells, colorectal cells, ductal cells, lobular cells, lymphocytes, retinal cells, photoreceptor cells or cochlear cells.

In some embodiments the reporter iPS cells comprises a multicistronic reporter as described in any of the embodiments above, wherein the multicistronic reporter is driven by the promoter which is active only in pluripotent cells. For example the promoter operably linked to the open reading frame comprising two or more cistrons, can be Oct-4, Sox2, Nanog, KLF4, TRA-1-60, TRA-2-54, TRA-1-81, SSEA1, SSEA4 or the promoter of any pluripotency associated gene.

In some embodiments the reporter iPS cell comprises a multicistronic reporter wherein the multicistronic reporter is driven by a promoter which is specific to a stage of differentiation. For example, the reporter iPS cell can be customized so that the promoter driving the multicistronic promoter is specific to a stage of differentiation. For example, using an OCT-4 promoter would allow expression of the fusion proteins encoded by the multicistronic construct to only be expressed in pluripotent cells. Using an MSP1+promoter would allow expression of the fusion proteins encoded by the multicistronic construct to only be expressed in pre-cardiac progenitor cells. Using an alpha-MHC promoter would allow expression of the fusion proteins encoded by the multicistronic construct to be expressed only in cardiomyocytes. Similarly using a lineage specific promoter to drive the expression of the fusion proteins encoded by a multicistronic construct of any of the embodiments described above, allows the user to monitor expression and movement of the fusion proteins within a cell of interest even in heterologous cell populations.

In some embodiments, the invention provides multireporter cells comprising multicistronic reporter vectors that encode reporter polypeptides that may be used to identify the lineage or cell type in which a particular multicistronic reporter vector is pressed in some embodiments, the multicistronic reporter vector comprises nucleic acid encoding a polypeptide (e.g., a “housekeeping” polypeptide) fused to a reporter polypeptide and operably linked to a tissue specific, lineage specific or sublineage specific promoter. In some embodiments, the nucleic acid encoding the polypeptide is encoded as part of the multicistronic open reading frame of the vector. Here, the tissue-specific, lineage-specific or sublineage specific promoter drives expression of the multicistronic open reading frame in other embodiments, the tissue-specific, lineage-specific or sublineage specific promoter and reporter polypeptide are present on the multicistronic vector as a separate transcription unit from the multicistronic open reading frame. In some embodiments, an insulator region is present between the separate transcription unit and the multicistronic open reading frame in some embodiments, the separate transcription unit is 5′ to the multicistronic open reading frame. In some embodiments, the separate transcription unit is 3′ to the multicistronic open reading frame.

In some embodiments, the invention provides multireporter cells comprising multicistronic reporter vectors as described above, wherein the vector comprises nucleic acid encoding H2B fused to a reporter polypeptide and operably linked to an MLC2v promoter, an SLN promoter, or a SHOX2 promoter, thereby enabling expression of a reporter in a cardio subtype cell. In some embodiments, the invention provides multireporter cells comprising multicistronic reporter vectors as described above, wherein the vector comprises nucleic acid encoding H2B fused to a reporter polypeptide and operably linked to a vGAT promoter, a TH promoter, a GFAP promoter, or a vGLUT promoter, thereby enabling expression of a reporter in a neural subtype cell.

In some embodiments the reporter iPS cells comprises a multicistronic reporter wherein the multicistronic reporter is driven by a lineage specific promoter in some embodiments the lineage specific promoter is a promotor active in early endodermal, early mesodermal, early ectodermal, chorionic, or trophectoderm lineage. In some embodiments the lineage specific promoter is a promoter only active in progenitor cells. In some embodiments the lineage specific promoter is a promoter only active in a hematopoietic cell, an endothelial progenitor cell, a mesenchymal progenitor cell, a neural progenitor cell, an osteochondral progenitor cell, a lymphoid progenitor cell or a pancreatic progenitor cell. In some embodiments the multireporter cell is a cord endothelial cell, a cord blood stem cell, an adipose-derived stem cell, a hepatocyte, a keratinocyte, a neural stem cell, a pancreatic beta-cell, a lymphocyte progenitor cell or an amniotic cell.

In some embodiment the lineage specific promoter is a promoter of a cell derived from extraembryonic tissue, ectoderm, endoderm or mesoderm. In some embodiments the lineage specific promoter is a promoter that is expressed in cardiomyocytes, endothelial cells, neuronal cells, GABAergic neurons, astrocytes, dopaminergic neurons, glutamatergic neurons, hepatocytes, hepatoblasts, skeletal myoblasts, macrophages, cortical neurons, atrial cardiomyocytes, ventricular cardiomyocytes, nodal cardiomyocytes, Purkinje fibers, basal cells, squamous cells, renal cells, pancreatic beta cells, epithelial cells, mesenchymal cells, adrenocortical cells, osteoblasts, osteocytes, chondroblasts, chondrocytes, gastrointestinal cells, colorectal cells, ductal cells, lobular cells, lymphocytes, retia cells, photoreceptor cells or cochlear cells.

Using lineage specific promoters to dive expression of cistrons within the multicistronic constructs, can be used to identify cells of different lineages, to sort cells of different lineages, to test toxicity in cells of the different lineages, to test and monitor the effects of various molecules in cells of different lineage, to test effects of different therapies in cells of different lineages or to monitor movement of proteins in cells of different lineage in response to a stimulus. Example of molecules and therapies include chemicals, chemical compositions, small biologics, nanoparticles, peptides, antibodies, vaccines and combinations thereof.

In some embodiments a cell may comprise multiple multicistronic constructs, each driven by a different promoter. For example a cell may comprise a multicistronic construct wherein expression of the cistrons is driven by a first promoter, which might be a lineage specific promoter, and a second multicistronic construct driven by a second promoter. In some embodiments a multireporter cell may comprise one or more, two or more, three or more lineage specific promoters driving expression of cistrons in the multicistronic construct.

Toxicity can be tested by monitoring expression and, or movement within a cell or between cells of various peptides associated with toxicity. For example expression and movement of proteins involved in unfolded protein response, autophagy, DNA damage, oxidative stress and p53-dependent stress response.

A multireporter cell may be used to test the toxicity, to test and monitor the effects of various molecules in cells, to test effects of different therapies in cells or to monitor movement of proteins in cells in response to a stimulus. Example of molecules and therapies include chemicals, chemical compositions, small biologics, nanoparticles, peptides, antibodies, vaccines and combinations thereof.

In some embodiments, the invention provides for two or more multireporter cells. In some embodiments the two or more multireporter cells are co-cultured. For example, the two or more multireporter cells are co-cultured as a cellular model. In some embodiments, the two or more reporter cells are co-cultured as a three-dimensional (3-D) cell model. Examples of 3-D models include but are not limited to tumor models, vascular networks, bioprinted cells, and tissue models. In some embodiments, the two or more multireporter cells comprise at least one pluripotent cell. In some embodiments, the two or more multireporter cells comprise at least one iPSC. In some embodiments, the cellular model comprises multireporter cells derived from one or more iPSC. In some embodiments, at least one multireporter cell in the co-culture of two or more multireporter cells comprises it reporter polypeptide operably linked to a lineage specific promoter, a sublineage specific promoter or a tissue specific promoter. In some embodiments reporter polypeptide operably linked to a lineage specific promoter, a sublineage specific promoter or tissue specific promoter is used to identify the cell or lineage of a cell in the cellular model.

In some embodiments, the invention provides or two or more cells that are co-cultured, wherein at least one of the cells is a multireporter cell. For example, the two or more cells are co-cultured as a cellular model. In some embodiments, the two or more cells are co-cultured as a three-dimensional (3-D) cell model Examples of 3-D models include but are not limited to tumor models, vascular networks, bioprinted cells, and tissue models. In some embodiments, the two or more cells comprise at least one multireporter pluripotent cell. In some embodiments, the at least one multireporter cell is an iPSC. In some embodiments, the cellular model comprises multireporter cells derived from one or more iPSC. In some embodiments, at least one multireporter cell in the co-culture of two or more cells comprises a reporter polypeptide operably linked to a lineage specific promoter, a sublineage specific promoter or a tissue specific promoter. In some embodiments reporter polypeptide operably linked to a lineage specific promoter, a sublineage specific promoter or tissue specific promoter is used to identify the cell or lineage of a cell in the cellular model. In some embodiments, the invention provides a cellular model comprising at least one, two, three, four, five, or more than five multireporter cells.

In some embodiments, the invention provides methods for generating a multireporter cell, the method comprising introducing the multicistronic reporter vector described herein into any acceptor cell as described herein. In some embodiments, the multispecific reporter vector is inserted into the acceptor site of the acceptor cell recombinase system. In some embodiments, the multicistronic reporter vector comprises a recombinase associated nucleic acid which can insert into a recombinase associated nucleic acid of the acceptor cell by way of a recombinase protein in the acceptor cell. In some embodiments, the nucleic acid encoding the recombinase protein is stably introduced in the acceptor cell. In some embodiments, the nucleic acid encoding the recombinase is transiently introduced into the acceptor cell. In some embodiments, the nucleic acid encoding the recombinase is transiently introduced into the acceptor cell before, at the same time or after introduction of the multicistronic reporter vector. In some embodiments, the recombinase protein is introduced into the acceptor cell. In some embodiments, the recombinase associated nucleic acid sequence is FRT nucleic acid sequence and the acceptor cell comprises a FLT recombinase. In some embodiments, the recombinase associated nucleic acid is attP and the acceptor cell comprises a Bxb1 recombinase, a PhiC31 recombinase, or R4 recombinase. In some embodiments, the recombinase associated nucleic acid sequence is loxP nucleic acid sequence and the acceptor cell comprises a CRE recombinase.

In some embodiments, the multicistronic reporter construct is integrated at a first specific cite in the genome of the multireporter stem cell. In some embodiments, the multireporter stem cell of the invention further comprises a nucleic acid integrated at a second specific cite in the genome of the multireporter stem cell. In some embodiments, the nucleic acid integrated at the second specific cite in the genome of the multireporter stem cell encodes a polypeptide, a reporter polypeptide, a cytotoxic polypeptide, a selectable polypeptide, a constitutive Cas expression vector or inducible Cas expression vector, a constitutive Cas9 expression vector or inducible Cas9 expression vector. In some embodiments, the Cas (e.g., Cas9) expression vector integrated in the second cite of the multireporter cell can be used in a sgRNA library screening and validation, either individually or in pools. In some embodiments, the nucleic acid integrated at the second specific cite in the genome of the multireporter stem cell comprises a second multicistronic reporter construct

### Libraries

In some aspects, the invention provides one or more libraries of multicistronic reporter vectors, wherein the library comprises multicistronic reporter molecules comprising different transgenes fused to reporter polypeptides, wherein two or more of the different transgene, on each vector are operably linked to a lineage specific promoter and are expressed essentially 1:1 stoichiometrically when introduced to cells Rach vector in the library comprises a reporter polypeptide operably linked to a lineage-specific promoter to serve as a barcode to identify the lineage of the recipient cell of a particular vector of the library. In some embodiments, the library comprises combinations of lineage-specific barcodes that are permuted differently for each cell lineage. This enables development of assays with pooled populations of different cell lineages, that can be identified and monitored through their unique fluorescent barcode and enables the development of more physiologically relevant assays with co-culture of mixed cell lineages.

In some embodiments, the library comprises reporter vectors that encode one or more transgenes encode polypeptides that can be used to profile or distinguish a single or multiple biological pathways, cross-talk between two or more biological pathways, synthetic lethality, cellular homeostasis, organelle homeostasis, cell-cell interactions, a toxicity response or other cellular or subcellular phenotypes. In some embodiments, the library comprises reporter vectors that encode one or more transgenes that encode polypeptides that can be used to profile phenotypic features of a coil. In some embodiments, the library comprises two or more different multicistronic reporter vectors. In some embodiments, the library comprises between any of about two and about 10, about 10 and about 20, about 20 and about 30, about 30 and about 40, about 40 and about 50, about 50 and about 100, about 100 and about 500, about 500 and about 100, about 1000 and about 10,000 different multicistronic reporter vectors. In some embodiments, the library comprises greater than about 10,000 different multicistronic reporter vectors.

In some embodiments, the library comprises multicistronic reporter vectors to profile a biological pathway or phenotype associated with a disease. In some embodiments, the disease is cancer, a cardiovascular disease, a neurodegenarative disease or an autoimmune disease. In some embodiments, the biological pathway or phenotype is a pathway or phenotype associated with toxic response mechanism within the cell. In some embodiments, the biological pathway or phenotype is a pathway or phonotype associated with aging. In some embodiments, the library comprises multicistronic reporter vectors to profile biological pathway associated with cell proliferation, cell differentiation, cell death, apoptosis, autophagy. DNA damage and repair, or oxidative stress, chromatin/epigenetics (e.g. chromatin acetylation). MAPK signaling (e.g. MAPK/Erk), PI3K/Akt signaling (e.g. mTor signaling), translational control (e.g. eIF2 regulation), cell cycle and checkpoint control (G1/S checkpoint), cellular metabolism (e.g. insulin receptor signaling), development and differentiation signaling (e.g. Wnt signaling), immunology and inflammation signaling (e.g. JAK/STAT signaling), tyrosine kinase signaling (e.g. ErbB/HER signaling), vesicle trafficking, cytoskeletal regulation or protein degradation (e.g. ubiquitin pathway) and any synthetically lethal combinations of these pathways. In some embodiments, each multicistronic vector comprising transgenes used to profile a specific single biological pathway, specific cross-talk between two or more biological pathways, a specific cellular homeostasis, a specific organelle homeostasis or a specific toxicity response of the library comprises a common transgene fused to reporter polypeptide.

In some embodiments, the invention provides libraries of acceptor cells for receiving multicistronic reporter vectors. In some embodiments, the library comprises two or more deferent acceptor cells as described herein. In some embodiments, the library comprises between any of about two and about 10, about 10 and about 20, about 20 and about 30, about 30 and about 40, about 40 and about 50, about 50 and about 100, about 100 and about 500 different acceptor cells. In some embodiments, the library comprises mom than about 300 different acceptor cells. In some embodiments, a common multicistronic reporter vector can be introduced into two or more acceptor cells to compare profiles in different cellular backgrounds.

In some embodiments, the invention provides libraries of multireporter coils, wherein each cell ti the library comprises a multicistronic reporter vector comprising different transgenes fused to reporter polypeptides, wherein the different transgenes on each vector are expressed essentially 1:1 stoichiometrically when introduced to cells. Each vector in library comprises a reporter poly peptide operably linked to a lineage-specific promoter to serve as a barcode to identify the lineage of the recipient cell of a particular vector in the library of cells. In some embodiments, the library of multireporter cells comprises a mixed population of cells of different lineages. In some embodiments, the library comprises combinations of lineage-specific barcodes that are permeated differently for each cell lineage to allow identification of the lineage of different cells in the library. In some embodiments, the library comprises between any of about two and about 10, about 10 and about 20, about 20 and about 30, about 304 and about 40, about 40 and about 50, about 50 and about 100, about 100 and about 500 deferent multireporter cells. In some embodiments, different multicistronic reporter vectors that target a common pathway share a common reporter polypeptide as a means for identifying cells that received related multicistronic reporter vectors.

In some embodiments, the library of acceptor cells and/or the library of multireporter cells comprises different pluripotent, multipotent and/or progenitor cells. In some embodiments, the different pluripotent or multipotent cells include one or more of an induced pluripotent stein cell, a multipotent cell, a hematopoietic cell, an endothelial progenitor acceptor cell, a mesenchymal progenitor cell, a neural progenitor cell, an osteochondral progenitor cell, a lymphoid progenitor cell or a pancreatic progenitor cell. In some embodiments, the library of pluripotent or multipotent cells multireporter cells is differentiated after introduction of the multicistronic reporter vector. In some embodiments, the library includes one or more of a WTC-11 IPSC or an NCRM5 iPSC.

In some embodiments, the invention provides a library or two or more multireporter cells wherein the two or mom multireporter coils are co-cultured. For example, the two or more multireporter cells of the library are co-cultured as a cellular model. In some embodiments, the two or more reporter cells of the library are co-cultured as a three-dimensional (3-D) cell model. Examples of 3-D models include but are not limited to tumor models, vascular networks, bioprinted ceils, and tissue models. In some embodiments, the two or more multireporter ceils comprise at least one pluripotent cell. In some embodiments, the two or more multireporter cells comprise at least one iPSC. In some embodiments, the cellular model comprises multireporter cells derived from one or more iPSC. In some embodiments, at least one multireporter cell in the co-culture of two or more multireporter cells comprises a reporter polypeptide operably linked to a lineage specific promoter, a sublineage specific promoter or a tissue specific promoter. In some embodiments reporter polypeptide operably liked to a lineage specific promoter, a sublineage specific promoter or tissue specific promoter is used to identify the cell or lineage of a cell in the cellular model. In some embodiments, the invention provides a library or two or more cells that are co-cultured wherein at least one of the cells is a multireporter cell. In some embodiments, the invention provides a library of cellular models wherein the cellular model comprises at least one, two, three, four, five, or more than five multireporter cells.

In some embodiments, each cell in the library of multireporter cells comprises the same multicistronic repo ter vector. In other embodiments, cells in the library of multireporter cells comprise different multicistronic reporter vectors. In some embodiments, the different multicistronic reporter vectors were introduced to isogenic acceptor cells.

In some embodiments, the invention provides libraries of multireporter cells wherein the reporter cells comprise a multicistronic reporter vectors encoding one or more polypeptides fused to a reporter polypeptide, and operably linked to a lineage specific promoter, that can be used to profile or distinguish a single or multiple biological pathways, cross-talk between two or more biological pathways, synthetic lethality, cellular homeostasis, organelle homeostasis, cell-cell interactions, a toxicity response or other cellular or subcellular phenotypes. In some embodiments, the biological pathway is a pathway associated with a disease. In some embodiments, the disease is cancer, a cardiovascular disease, a neurodegenerative disease or an autoimmune disease. In some embodiments, the biological pathway is a pathway associated with toxic response mechanism within the cell. In some embodiments, the biological pathway or phenotype is a pathway or phenotype associated with aging. In some embodiments, the biological pathway is a pathway associated with cell proliferation, cell differentiation, cell death, apoptosis, autophagy, DNA damage and repair, or oxidative stress, chromatin/epigenetics (e.g. chromatin acetylation), MAPK signaling (e.g. MAPK/Erk), PI3K/Akt signaling (e.g. mTor signaling), translational control (e.g. eIF2 regulation), cell cycle and checkpoint control (G1/S checkpoint), cellular metabolism (e.g. insulin receptor signaling), development and differentiation signaling (e.g. Wnt signaling), immunology and inflammation signaling (e.g. JAK/STAT signaling), tyrosine kinase signaling (e.g. ErbB/HER signaling), vesicle trafficking, cytoskeletal regulation or protein degradation (e.g. ubiquitin pathway) and synthetically lethal combinations of these pathways. In some embodiments, the library of multireporter cells comprise different multicistronic vector comprising transgenes used to profile a specific single biological pathway, specific cross-talk between two or more biological pathways, a specific cellular homeostasis, a specific organelle homeostasis or a specific toxicity response wherein each different multicistronic reporter vectors comprises a common transgene fused to nucleic acid encoding a reporter polypeptide. In some embodiments, the common transgene product fused to a reporter polypeptide is used as a means for identifying cells that received related multicistronic reporter vectors. In some embodiments, the common transgene product fused to a reporter polypeptide is used as a means for identifying the type, lineage or sublineage of cells expressing the multicistronic reporter vector.

### Assays

The invention provides live cell assays using the cells and vectors described herein. In some embodiments, the assay is performed on a single live cell. In some embodiments, the invention provides a method of profiling two or more polypeptides in a live cell, the method comprising determining the expression mid/or location of the two or more of the transgenes of a multireporter, lineage barcoded cell as described herein. In some embodiment, the method is used to profile or distinguish a single or multiple biological pathways, cross-talk between two or more biological pathways, synthetic lethality, cellular homeostasis, organelle homeostasis, cell-cell interactions, a toxicity response or other cellular or subcellular phenotypes. In some embodiments, the method is used to profile phenotypic fixtures of a cell. In some embodiments, the expression and/or location of the two or more of the transgenes is determined at one or more time points. In some embodiments, the expression and/or location of the two or more of the transgenes is determined at one or more of 1 minute, 5 minutes, 10 minutes, 15 minutes, 30 minutes, 45 minutes, 1 hour, 2 hours, 3 hours, 4 hours, 5 hours, 6 hours, 12 hours, 24 hours, 2 days, 4 days, 7 days, 14 days, 21 days, 30 days, 1 month, 3 month, 6 month, 9 month, 1 year, or any time therebetween or more than 1 year after initiation of the analysis.

In some embodiments, the invention provides methods to measure the effects of an agent on the profile of two or more polypeptides m a live cell, the method comprising subjecting a multireporter, lineage barcoded cell as described herein to the agent and determining the expression and/or location of the two or more transgenes in the cell in response to the agent. In some embodiments, the agent is a drug or drug candidate. In some embodiments, the agent is a cancer drug or cancer drug agent. In some embodiments, the method is a toxicology screen.

In some embodiments of the above assays and methods, the profile is obtained from a single live cell. In some embodiments, the profile is determined for multiple live cells. In some embodiments, the cells are culture on a tissue culture plate, including but not limited to multiwell tissue culture plates such as a 96-well or a 384-well tissue culture plate. In some embodiments, the cells are in a suspension culture.

In some embodiments of the above assays and methods, determining the expression and/or location of the two or more transgenes is performed in a library of multireporter, lineage barcoded cells.

In some embodiments, the expression and/or location of the two or more polypeptides is measured by microscopy, high throughput microscopy, fluorescence-activated cell sorting (FACS), luminescence, using a plate reader, mass spectrometry, or deep sequencing.

In some embodiments, the invention provides pooled assays in which cells of different lineages are pooled and used in the assay. In some embodiments, stem cells with barcoded lineages are used in an assay with pooled cells of different lineages. In some embodiments, lineage-specific reporter polypeptides am permuted differently for each cell lineage thereby enabling development of assays with pooled populations of cells of different lineages that can be identified and monitored through their unique fluorescent barcode. This enables the development of mom physiologically relevant assays with co-culture of mixed cell lineages, in contrast to assays based on purified populations of a single cell lineage.

In some embodiments, the invention provides multicistronic reporter vectors that enable lineage specific labeling. In some embodiments, the multicistronic reporter vector encodes reporter polypeptides that may be used to identify the lineage or cell type in which a particular multicistronic reporter vector is expressed. In some embodiments, the multicistronic reporter vector comprises nucleic acid encoding a polypeptide (e.g., a “housekeeping” polypeptide) fused to a reporter polypeptide and operably linked to a tissue specific, lineage specific or sublineage specific promoter. In some embodiments, the nucleic acid encoding the polypeptide is encoded as part of the multicistronic open reading frame of the vector. Here, the tissue-specific, lineage-specific or sublineage specific promoter drives expression of the multicistronic open reading frame. In other embodiments, the tissue-specific, lineage-specific or sublineage specific promoter and reporter polypeptide are present on the multicistronic vector as a separate transcription unit from the multicistronic open reading frame. In some embodiments, an insulator region is present between the separate transcription unit and the multicistronic open reading frame. In some embodiments, the separate transcription unit is 5′ to the multicistronic open reading frame. In some embodiments, the separate transcription unit is 3′ to the multicistronic open reading frame.

In some embodiments, the vector comprises nucleic acid encoding H2B fused to a reporter polypeptide and operably linked to an MLC2v promoter, an SLN promoter, or a SHOX2 promoter, thereby enabling expression of a reporter in a cardiomyocyte subtype cell. In some embodiments, the vector comprises nucleic acid encoding H2B fused to a reporter polypeptide and operably linked to a vGAT promoter, a TH promoter, a GFAP promoter, or a vGLUT promoter, thereby enabling expression of a reporter in a neural subtype cell

### Kits and Articles of Manufacture

In some embodiments, the invention provides a kit comprising one or more multicistronic reporter vectors as described herein. In some embodiments, the invention provides a kit comprising one or more acceptor cells as described herein. In some embodiments, the invention provides a kit comprising one or more of the multireporter cells described herein. In some embodiments, the invention provides a kit comprising one or more multicistronic reporter vectors described herein and one or more acceptor cells as described herein. In some embodiments, the kit further comprises instructions for using the multicistronic reporter vectors, acceptor cells and/or multireporter cells described herein. In some embodiments, the kit comprises a mixture of isogenic, but differentially labeled, multireporter cells. Such cells enable directed plating of a pooled assay.

In some embodiments, the kit comprises a library or two or more multireporter cells wherein the two or more multireporter cells are co-cultured. For example, the two or more multireporter cells of the kit may be co-cultured as a cellular model. In some embodiments, the two or more reporter cells of the kit are co-cultured as a three-dimensional (3-D) cell model. Examples of 3-D models include but are not limited to tumor models, vascular networks, bioprinted cells, and tissue models. In some embodiments, the kit comprises cultured cells that have formed a cellular model. In other embodiments, the kit comprises individual cells which can be combined and co-cultured to form a cellular model. In some embodiments, the two or more multireporter cells of the kit comprise at least one pluripotent cell. In some embodiments, the two or more multireporter cells of the kit comprise at least one iPSC in some embodiments, the cellular model of the kit comprises multireporter cells derived from one or mow iPSC In some embodiments, at least one multireporter ell in the kit comprises a reporter polypeptide operably linked to a lineage specific promoter, a sublineage specific promoter or a tissue specific promotor. In some embodiments reporter polypeptide operably linked to a lineage specific promoter, a sublineage specific promoter or tissue specific promoter is used to identify the cell or lineage of a cell in the cellular model. In some embodiments, the invention provides a kit comprising two or more cells that are co-cultured wherein at least one of the cells is a multireporter cell. In some embodiments, the invention provides a kit comprising a cellular model or cells for generating a cellular model wherein the cellular model comprises at least one, two, three, four, five, or mere than five multireporter cells.

In some embodiments, the invention provides a library of acceptor cells and/or reporter cells arrayed in a multiwell plate (e.g., a 96 well plate or 34 well plate). In some embodiments, the invention provides a library of cellular models as described above arrayed in a multiwell plate. In some embodiments, the cells in the multiwell plate are cryopreserved.

The multicistronic reporter vectors, acceptor cells and/or multireporter cells described herein may be contained within an article of manufacture. The article of manufacture may comprise a container containing the multicistronic reporter vectors, acceptor cells and/or multireporter cells described herein. In some embodiments, the article of manufacture comprises: (a) a container comprising multicistronic reporter vectors, acceptor cells and/or multireporter cells described herein within the container; and (b) a package insert with instructions for using the multicistronic reporter vectors, acceptor cells and/or multireporter cells described herein.

In some embodiments, the article of manufacture comprises a container and a label or package insert on or associated with the container. Suitable containers include, for example, bottles, vials, syringes, etc. The containers may be formed from a variety of materials such as glass or plastic. The article of manufacture may further include other materials desirable from a commercial and user standpoint, including other buffers, diluents, reagents, tissue culture media, filters, needles, and syringes.

In some embodiments, the invention provides a kit comprising a library of acceptor cells or reporter cells arrayed in a multiwell plate. In some embodiments the cells are plated cryopreserved.

### Exemplary Embodiments

Embodiment 1. A multicistronic reporter vector comprising:
a promoter operably linked to an open reading frame, wherein the promoter is a lineage-specific promoter, wherein the open reading frame comprises two or more cistrons, and wherein expression of the open reading frame in a cell yields separate component polypeptide products from each cistron;
wherein each cistron comprises a multiple cloning site (MCS) and nucleic acid encoding a reporter vector, wherein each cistron encodes a different reporter polypeptide; and
wherein expression of two or more nucleic acids encoding polypeptides inserted into the two or more multiple cloning sites and fused to the reporter polypeptides is essentially at about 1:1 stoichiometry.

Embodiment 2. A multicistronic reporter vector comprising:
a first promoter linked to a transactivator polypeptide, wherein the first promoter is a lineage-specific promoter,
a second promoter operably linked to an open reading frame, therein the second promoter is inducible by the transactivator polypeptide, wherein the open reading frame comprises two or more cistrons, and wherein expression of the open reading frame in a cell yields separate component polypeptide products from each cistron;
wherein each cistron comprises a multiple cloning site (MCS) and nucleic acid encoding a reporter vector, wherein each cistron encodes a different reporter polypeptide; and wherein expression of two or more nucleic acids encoding polypeptides inserted into the two or more multiple cloning sites and fused to the reporter polypeptides is essentially at about 1:1 stoichiometry.

Embodiment 3. The multicistronic reporter vector of embodiment 2, wherein the transactivator polypeptide is a tetracycline transactivator polypeptide and the second promoter comprises a tetracycline responsive element.

Embodiment 4. The multicistronic reporter vector of embodiment 3, wherein the tetracycline responsive element is a Tet operator 2 (TetO2) inducible or repressor element.

Embodiment 5. A multicistronic reporter vector comprising:
a first promoter linked to a nucleic acid encoding an organelle-specific polypeptide, wherein the first promoter is a lineage-specific promoter;
a second promoter operably linked to an open reading frame, wherein the second promoter is a constitutive promoter, wherein the open reading flame comprises two or more cistrons, and wherein expression of the open reading frame in a cell yields separate component polypeptide products from each cistron.
wherein each cistron comprises a multiple cloning site (MCS) and nucleic acid encoding a reporter vector, wherein each cistron encodes a different reporter polypeptide; and
wherein expression of two or more nucleic acids encoding polypeptides inserted into the two or more multiple cloning sites and fused to the reporter polypeptides is essentially at about 1:1 stoichiometry.

Embodiment 6. The multicistronic reporter vector of embodiment 5, wherein the organelle-specific polypeptide is H2B.

Embodiment 7. The multicistronic reporter vector of embodiment 5 or 6 wherein the constitutive promoter is a Cytomegalovirus a (CMV), a Thymidine Kinase (TK), an eF1-alpha, a Ubiquitin C (UbC), a Phosphoglycerate Kinase (PGK), a CAG promoter, an SV40 promoter, or a human β-actin promoter.

Embodiment 8. The multicistronic reporter vector of any one of embodiments 5-7, wherein the promoter comprises a tetracycline responsive element.

Embodiment 9. The multicistronic reporter vector of embodiment 8, wherein the tetracycline responsive element is a Tet operator 2 (TetO2) inducible or repressor element.

Embodiment 10. The multicistronic reporter vector of any one of embodiments 2-9, wherein the first promoter and the second promoter are in different orientations.

Embodiment 11. The multicistronic reporter vector ofany one of embodiments 2-10, wherein the first promoter and the second promoter are separated by an insulator nucleic acid.

Embodiment 12. The multicistronic reporter vector of any one of embodiments 1-11, wherein the cistrons are separated from one another by nucleic acid encoding one or more self-cleaving peptide and/or one or more internal ribosome entry site (IRES).

Embodiment 13. The multicistronic reporter vector of embodiment 12, wherein the one or more self-cleaving peptides is a viral self-cleaving peptide.

Embodiment 14. The multicistronic reporter vector of embodiment 13, wherein the one or more viral self-cleaving peptides is one or more 2A peptides.

Embodiment 15. The multicistronic reporter vector of embodiment 14, wherein one or more 2A peptides is a T2A peptide, a P2A peptide, an E2A peptide or a F2A peptide.

Embodiment 16. The multicistronic reporter vector of any one of embodiments 12-15, wherein the reporter polypeptide further comprises one or more nucleic acids encoding a peptide linker between one or more of the reporter polypeptides and one or more of the self-cleaving peptides.

Embodiment 17. The multicistronic reporter vector of embodiment 16, wherein the peptide linker comprises the sequence Gly-Ser-Gly.

Embodiment 18. The multicistronic reporter vector of any one of embodiments 1-17, wherein the reporter polypeptide is a fluorescent reporter polypeptide.

Embodiment 19 The multicistronic reporter vector of any one of embodiments 1-18, wherein the reporter polypeptide for each cistron is selected from GFP, EGFP, Emerald, Citrine, Venus, mOrange, mCherry, TagBFP, mTurquoise, Cerulean, UnaG, dsRed, eqFP611, Dronpa, RFP, TagRFPs, TdTomato, KFP, EosFP, Dendra, IrisFP, iRFP and smURFP.

Embodiment 20. The multicistronic reporter vector of any one of embodiments 1-19, wherein the open reading frame comprises a first cistron and a second cistron, wherein each cistron comprises 5′ to 3′ nucleic acid comprising a MCS, nucleic acid encoding a reporter polypeptide, and nucleic acid encoding a linker peptide; wherein the first cistron and the second cistron are separated by nucleic acid encoding a viral cleavage peptide.

Embodiment 21. The multicistronic reporter vector of any one of embodiments 1-19, wherein the open reading frame comprises a first cistron, a second cistron and a third cistron, wherein each cistron comprises 5′ to 3′ nucleic acid comprising a MCS, nucleic acid encoding a reporter polypeptide, and nucleic acid encoding a linker peptide; wherein the first cistron and the second cistron are separated by nucleic acid encoding a first viral cleavage peptide and the second cistron and the third cistron are separated by nucleic acid encoding a second viral cleavage peptide.

Embodiment 22. The multicistronic reporter vector of any one of embodiments 1-19, wherein the open reading frame comprises a first cistron, a second cistron, a third cistron and a fourth cistron, wherein each cistron comprises 5′ to 3′ nucleic acid comprising a MCS, nucleic acid encoding a reporter polypeptide nucleic acid encoding a linker peptide; wherein the first cistron and the second cistron are separated by nucleic acid encoding a first viral cleavage peptide, the second cistron and the third cistron are separated by nucleic acid encoding a second viral cleavage peptide the third cistron and the fourth cistron are separated by nucleic acid encoding a third viral cleavage peptidic.

Embodiment 23. The multicistronic reporter vector of any one of embodiments 1-19, wherein the vector comprises a promoter operably linked to an open reading frame, wherein the open reading frame comprises a first cistron, a second cistron, a third cistron and a fourth cistron, wherein each cistron comprises 5′ to 3′ nucleic acid comprising a MCS, nucleic acid encoding a reporter polypeptide, nucleic acid encoding a linker peptide; wherein the first cistron and the second cistron are separated by nucleic acid encoding a first viral cleavage peptide, the second cistron and the third cistron are separated by nucleic acid encoding a second viral cleavage peptide the third cistron and the fourth cistron an separated by nucleic acid encoding an IRES.

Embodiment 24. The multicistronic reporter vector of any one of embodiments 1-23, wherein the lineage-specific promoter is specific for cells of heart, blood, muscle, lung, liver, kidney, pancreas, brain, or skin lineage.

Embodiment 25. The multicistronic reporter vector of any one of embodiments 1-24, wherein the lineage specific promoter is a sublineage-specific promoter.

Embodiment 26. The multicistronic reporter vector of any one of embodiments 1-25, wherein the lineage-specific promoter is a cardiac specific promoter.

Embodiment 27. The multicistronic reporter vector of embodiment 26, wherein the cardiac-specific promoter is a MCLV2v, a SLN, a SHOX2, a MYBPC3, a TNNI3 or an α-MHC promoter.

Embodiment 23. The multicistronic reporter vector of any one of embodiments 1-25, wherein the lineage-specific promoter is a neural specific promoter.

Embodiment 29. The multicistronic reporter vector of embodiment 28, wherein the neural-specific promoter is a vGAT, a TH, a GFAP, or a vGLUT1 promoter.

Embodiment 30. The multicistronic reporter vector of any one of embodiments 1-29, further comprising a site-specific recombinase sequence located 3′ to the open reading frame.

Embodiment 31. The multicistronic reporter vector of embodiment 30, wherein the vector further comprises nucleic acid encoding a selectable marker, wherein the nucleic acid encoding the selectable marker is not operably linked to the promoter when the site-specific recombinase sequence has not recombined and is operably linked to the promoter when the site-specific recombinase sequence recombines with its target site-specific recombinase sequence.

Embodiment 32. The multicistronic reporter vector of embodiment 31, wherein the site-specific recombinase sequence is a FRT nucleic acid sequence and/or an attP nucleic acid and/or a loxP nucleic acid sequence.

Embodiment 33. The multicistronic reporter vector of embodiment 31 or 32, wherein the selectable marker confers resistance to hygromyocin. Zeocin™, puromycin, neomycin or an analog of hygromyocin, Zeocin™, puromycin, blasticidin or neomycin.

Embodiment 34. The multicistronic reporter vector of any one of embodiments 1-33, wherein nucleic acid encoding one or more polypeptides is inserted in-frame into the one or more MCS.

Embodiment 35. The multicistronic reporter vector of any one of embodiments 1-34, wherein at least one cistron comprises nucleic acid encoding a housekeeping gene.

Embodiment 36. The multicistronic reporter vector of embodiment 35, wherein the housekeeping gene is H2B.

Embodiment 37. The multicistronic reporter vector of anyone of embodiments 1-36, wherein at least one cistron comprises nucleic acid encoding an organelle marker.

Embodiment 38. The multicistronic reporter vector of embodiment 37, wherein the organelle marker comprises H2B, α-actinin 2 or a mitochondrial targeting signal fused to the reporter polypeptide.

Embodiment 39. The multicistronic reporter vector of any one of embodiments 34-38, wherein the one or more polypeptides comprise polypeptides that cam be used to profile or distinguish a single or multiple biological pathways, cross-talk between two or more biological pathways, synthetic lethality, cellular homeostasis, organelle homeostasis, other cellular or subcellular phenotypes, cell-cell interactions or a toxicity response.

Embodiment 40. A multireporter stem cell, wherein the multireporter stem cell comprises a multicistronic reporter construct, wherein the multicistronic reporter construct comprises
a promoter operably linked to an open reading frame, wherein the promoter is a lineage-specific promoter, wherein the open reading frame comprises two or more cistrons, and wherein expression of the open reading frame in a cell yields separate component polypeptide products from each cistron;
wherein each cistron comprises a multiple cloning site (MCS) and nucleic acid encoding a reporter vector, wherein each cistron encodes a different reporter polypeptide;
wherein expression of two or more nucleic acids encoding polypeptides inserted into the two or more multiple cloning sites and fused to the reporter polypeptides is essentially at about 1:1 stoichiometry; and
wherein the stem cell is a pluripotent stem cell, a multipotent stem cell or an induced pluripotent stem (iPS) cell.

Embodiment 41. A multireporter stem cell, wherein the multireporter stem cell comprises a multicistronic reporter construct, wherein the multicistronic reporter construct comprises
a first promoter linked to a transactivator polypeptide, wherein the first promoter is a lineage-specific promoter,
a second promoter operably linked to an open reading frame, wherein the second promoter is inducible by the transactivator polypeptide, wherein the open reading frame comprises two or more cistrons, and wherein expression of the open reading frame in a cell yields separate component polypeptide products from each cistron;
wherein each cistron comprises a nullable cloning site (MCS) and nucleic acid encoding a reporter vector, wherein each cistron encodes a different reporter polypeptide;
wherein expression of two or more nucleic acids encoding polypeptides inserted into the two or more multiple cloning sites and fused to the reporter polypeptides is essentially at about 1:1 stoichiometry; and
wherein the stem cell is a pluripotent stem cell, a multipotent stem cell or an induced pluripotent stem (iPS) cell.

Embodiment 42. A multireporter stem cell of embodiment 41, wherein the transactivator polypeptide is a tetracycline transactivator polypeptide and the second promoter comprises a tetracycline responsive element.

Embodiment 43. The multireporter stem cell of embodiment 42, wherein the tetracycline responsive element is a Tet operator 2 (TetO2) inducible or repressor element.

Embodiment 44. A multireporter stem cell, wherein the multireporter stem cell comprises a multicistronic reporter constrict wherein the multicistronic reporter construct comprises
a first promoter linked to a nucleic acid encoding a housekeeping polypeptide, wherein the first promoter is a lineage-specific promoter;
a second promoter operably linked to an open reading frame, wherein the second promoter is a constitutive promoter, wherein the open reading frame comprises two or more cistrons, and wherein expression of the open reading frame in a cell yields separate component polypeptide products from each cistron;
wherein each cistron comprises a multiple cloning site (MCS) and nucleic acid encoding a reporter vector, wherein each cistron encodes a different reporter polypeptide;
wherein expression of two or more nucleic acids encoding polypeptides inserted into the two or more multiple cloning sites and Fused to the reporter polypeptides is essentially at about 1:1 stoichiometry; and
wherein the stem cell is a pluripotent stem cell, a multipotent stem cell or an induced pluripotent stem (iPS) cell.

Embodiment 45. The multireporter stem cell of embodiment 44, wherein the housekeeping polypeptide is H2B.

Embodiment 46. The multireporter stem cell of embodiment 44 or 45, wherein the constitutive promoter is a Cytomegalovirus a (CMV), a Thymidine Kinase (TK), an eF1-alpha, a Ubiquitin C (UbC), a Phosphoglycerate Kinase (PGK), a CAG promoter, an SV40 promoter, or a human β-actin promoter.

Embodiment 47. A multireporter stem cell of any one of embodiments 44-46, wherein the promoter comprises a tetracycline responsive element.

Embodiment 43. The multireporter stem cell of embodiment 47, wherein the tetracycline responsive element is a Tet operator 2 (TetO2) inducible or repressor element.

Embodiment 49. The multireporter stem cell of any one of embodiments 40-448, wherein the first promoter and the second promoter are in different orientations.

Embodiment 50. The multireporter stem cell of any one of embodiments 40-49, wherein the first promoter and the second promoter are separated by an insulator nucleic acid.

Embodiment 51. The multireporter stem cell any one of embodiments 40-50, wherein the cistrons are separated from one another by nucleic acid encoding one or more self-cleaving peptide and/or one or more internal ribosome entry site (IRES).

Embodiment 52. The multireporter stem cell of embodiment 51, wherein the one or mom self-cleaving peptides is a viral self-cleaving peptide.

Embodiment 51. The multi reporter stem cell of embodiment 52, wherein the one or more viral self-cleaving peptides is one or more 2A peptides.

Embodiment 54. The multireporter stem cell of embodiment 53, wherein one or more 2A peptides is a T2A peptide, a P2A peptide, an E2A peptide or a F2A peptide.

Embodiment 55. The multireporter stem cell of any one of embodiments 51-54, wherein the reporter polypeptide further comprises one or more nucleic acids encoding a peptide linker between one or more of the reporter polypeptides and one or more of the self-cleaving peptides.

Embodiment 56. The multireporter stem cell of embodiment 55, wherein the peptide linker comprises the sequence Gly-Ser-Gly.

Embodiment 57. The multireporter stem cell of any one of embodiments 40-56, wherein the reporter polypeptide is a fluorescent reporter polypeptide.

Embodiment 58. The multireporter stem cell of any one of embodiments 40-57, wherein the reporter polypeptide for each cistron is selected from GFP, EGFP, Emerald, Citrine, Venus, mOrange, mCherry, TagBFP, mTurquoise, Cerulean, UnaG, dsRed, eqFP611, Dronpa, RFP, TagRFPs, TdTomato, KFP, EosFP, Dendra, IrisFP, iRFP and smURFP.

Embodiment 59. The multireporter stem cell of any one of embodiments 40-58, wherein the open reading frame comprises a first cistron and a second cistron, wherein each cistron comprises 5′ to 3′ nucleic acid comprising a MCS, nucleic acid encoding a reporter polypeptide, and nucleic acid encoding a linker peptide: wherein the first cistron and the second cistron are separated by nucleic acid encoding a viral cleavage peptide.

Embodiment 60. The multireporter stem cell of any one of embodiments 40-58, wherein the open reading frame comprises a first cistron, a second cistron and a third cistron, wherein each cistron comprises 5′ to 3′ nucleic acid comprising a MCS, nucleic acid encoding a reporter polypeptide, and nucleic acid encoding a linker peptide; wherein the first cistron and the second cistron are separated by nucleic acid encoding a first viral cleavage peptide and the second cistron and the third cistron are separated by nucleic acid encoding a second viral cleavage peptide.

Embodiment 61. The multireporter stem cell of any one of embodiments 40-60, wherein the open reading frame comprises a first cistron, a second cistron, a third cistron and a fourth cistron, wherein each cistron comprises 5′ to 3′ nucleic acid comprising a MCS, nucleic acid encoding a reporter polypeptide nucleic acid encoding a linker peptide; wherein the first cistron and the second cistron are separated by nucleic acid encoding a first viral cleavage peptide, the second cistron and the third cistron are separated by nucleic acid encoding a second viral cleavage peptide the third cistron and the fourth cistron are separated by nucleic acid encoding a third viral cleavage peptide.

Embodiment 62. The multireporter stem cell of any one of embodiments 40-60, wherein the vector comprises a promoter operably linked to an open reading frame, wherein the open reading frame comprises a first cistron, a second cistron, a third cistron and a fourth cistron, wherein each cistron comprises 5′ to 3′ nucleic acid comprising a MCS, nucleic acid encoding a reporter polypeptide, nucleic acid encoding a linker peptide; wherein the first cistron and the second cistron are separated by nucleic acid encoding a first viral cleavage peptide, the second cistron and the third cistron are separated by nucleic acid encoding a second viral cleavage peptide the third cistron and the fourth cistron are separated by nucleic acid encoding an IRES.

Embodiment 63. The multireporter stem cell of any one of embodiments 40-62, wherein the lineage-specific promoter is specific for cells of heart, blood, muscle, lung, liver, kidney, pancreas, brain, or skin lineage.

Embodiment 64. The multireporter stem cell of any one of embodiments 40-63, wherein the lineage specific promoter is a sublineage-specific promoter.

Embodiment 65. The multireporter stem cell of any one of embodiments 40-64, wherein the lineage-specific promoter is a cardiac specific promoter.

Embodiment 66. The multireporter stem cell of embodiment 65, wherein the cardiac-specific promoter is a MCLV2v, a SLN, a SHOX2, a MYBPC3, a TNNI3 or an α-MHC promoter.

Embodiment 67. The multireporter stem cell of any one of embodiments 40-64, wherein the lineage-specific promoter is a neural specific promoter.

Embodiment 68. The multireporter stem cell of embodiment 67, wherein the neural-specific promoter is a vGAT, a TH, a GFAP, or a vGLUT1 promoter.

Embodiment 69. The multireporter stem cell of any one of embodiments 40-68, wherein nucleic acid encoding one or mow polypeptides is inserted in-frame into the one or more MCS.

Embodiment 70. The multireporter stem cell of any one of embodiments 40-69, wherein at least one cistron comprises nucleic acid encoding an organelle-specific polypeptide.

Embodiment 71. The multireporter stem cell of embodiment 70, wherein the organelle-specific polypeptide is H2B.

Embodiment 72. The multireporter stem cell of anyone of embodiments 40-71, wherein at least one cistron comprises nucleic acid encoding an organelle marker.

Embodiment 73. The multireporter stem cell of embodiment 72, wherein the organelle marker comprises H2B, α-actinin 2 or a mitochondrial targeting signal fused to the reporter polypeptide.

Embodiment 74. The multireporter stem cell of any one of embodiments 69-73, wherein the one or more polypeptides comprise polypeptides that can be used to profile or distinguish a single ox multiple biological pathways, cross-talk between two or more biological pathways, synthetic lethality, cellular homeostasis, organelle homeostasis, other cellular or subcellular phenotypes, cell-cell interactions or a toxicity response ater differentiation of the stem cell.

Embodiment 75. The multireporter stem cell of embodiment 74, where the profiling is performed on a single cell.

Embodiment 76. The multireporter stem cell of any one of embodiments 40-75, wherein the reporter polypeptide can be visualized by microscopy, high throughput microscopy, fluorescence-activated cell sorting (FACS), luminescence, or using a plate reader.

Embodiment 77. The multireporter stem cell of any one of embodiments 40-76, wherein the reporter polypeptide is analyzed before, during or after differentiation of the stem cell.

Embodiment 78. The multireporter stem cell of any one of embodiments 40-77, wherein the multicistronic reporter construct is integrated at a first specific cite in the genome of the multireporter stem cell.

Embodiment 79. The multireporter stem cell of embodiment 78, further comprising a nucleic acid integrated at a second specific cite in the genome of the multireporter stem cell.

Embodiment 80. The multireporter stem cell of embodiment 79, wherein the nucleic acid integrated at the second specific cite in the genome of the multireporter stem cell encodes a polypeptide, a reporter polypeptide, a cytotoxic polypeptide, a selectable polypeptide, a constitutive Cas9 expression vector or inducible Cas9 expression vector.

Embodiment 81. A library of multireporter vectors, wherein the library comprises two or more multicistronic reporter vectors according to any one of embodiments 1-39, wherein the two or more multicistronic reporter vectors comprise different transgenes fused to reporter polypeptides, wherein two or more of the different transgenes on each vector are expressed at essentially 1:1 stoichiometry when introduced to cells.

Embodiment 82. A library of multireporter vectors, wherein the library comprises two or more multicistronic reporter vectors according to any one of embodiments 1-39, wherein the two or more multicistronic reporter vectors comprise different lineage-specific promotors operably linked to transgenes fused to different reporter polypeptides such that expression of the reporter polypeptides can distinguish the cell type based on the lineage specific promoter.

Embodiment 83. The library of multireporter vectors of embodiment 82, wherein the same transgene is operably linked to the different lineages specific promoters and different reporter polypeptides.

Embodiment 84. The library of multireporter vectors of embodiment 83, wherein the transgene encodes a housekeeping polypeptide or an organelle-specific polypeptide.

Embodiment 85 ne library of multireporter vectors of embodiment 84, wherein the transgenic encodes H1B, α-actinin 2 or a mitochondrial targeting signal.

Embodiment 86. The library of multireporter vectors of any one of embodiments 81-85, wherein the reporter vectors encode one or more transgenes that can be used to profile or distinguish a single or multiple biological pathways, cross-talk between two or mom biological pathways, synthetic lethality, cellular homeostasis, organic homeostasis, other cellular or subcellular phenotypes, cell-cell interactions or a toxicity response or other phenotypes after differentiation of the cell.

Embodiment 87. The library of multireporter vectors of any one of embodiments 81-86, wherein the biological pathway or phenotype is a pathway or phenotype associated with a disease.

Embodiment 88. The library of multireporter vectors of embodiment 87, wherein the disease is cancer, a cardiovascular disease, a neurodegenerative or neurological disease or an autoimmune disease.

Embodiment 89. The library of multireporter vectors of embodiment 87 or 88, wherein the biological pathway or phenotype is a pathway or phenotype associated with toxic response mechanism within the cell.

Embodiment 90. The library of multireporter vectors of embodiment 87 or 88, wherein the biological pathway or phenotype is a pathway or phenotype associated aging.

Embodiment 91. The library of multireporter vectors of embodiment 87 or 88, wherein the biological pathway is a pathway associated with cell proliferation, cell differentiation, cell survival, cell death, apoptosis, autophagy, DNA damage and repair, oxidative stress, chromatin/epigenetics, MAPK signaling, PI3K/Akt signaling, protein synthesis, translational control, protein degradation, cell cycle aid checkpoint control, cellular metabolism, development and differentiation signaling, immunology and inflammation signaling, tyrosine kinase signaling, vesicle trafficking, cytoskeletal regulation, ubiquitin pathway.

Embodiment 92. A library of multireporter cells, wherein each cell in the library comprises a multicistronic reporter sector according to any one of embodiments 1-39, wherein cells in the library comprise different multicistronic reporter vectors.

Embodiment 93. A library of multireporter cells comprising two or more multireporter cells according to any one of embodiments 40-80 wherein two or more multireporter cells in the library comprise different multicistronic reporter vectors.

Embodiment 94. The library of multireporter cells of embodiment 92 or 93, wherein each multicistronic reporter vector comprises a common transgene fused to a common reporter polypeptide operably linked to a common lineage specific promoter.

Embodiment 95. The library of multireporter cells of embodiment 92 or 93, wherein each multicistronic reporter vector comprises a common transgene fused to a different reporter polypeptide and operably linked to a different lineage specific promoter.

Embodiment 96. The library of multireporter cells of any one of embodiments 92-95, wherein the library comprises pluripotent multipotent and/or progenitor cells.

Embodiment 97. The library of multireporter cells of any one of embodiments 92-95, wherein the library comprises different pluripotent, multipotent and/or progenitor cells.

Embodiment 98. The library of multireporter cells embodiment 96 or 97, wherein the pluripotent or multipotent cells include one or more of an induced pluripotent stem cell, a multipotent cell, a hematopoietic cell, an endothelial progenitor acceptor cell, a mesenchymal progenitor cell, a neural progenitor cell, an osteochondral progenitor cell, a lymphoid progenitor cell or a pancreatic progenitor cell.

Embodiment 99. The library of multireporter cells any one of embodiments 95-98, wherein the pluripotent or multipotent cells are differentiated after introduction of the multicistronic reporter vector.

Embodiment 100. The library of multireporter cells of any one of embodiments 95-99, wherein different multicistronic reporter vectors were introduced to isogenic pluripotent or multipotent acceptor cells.

Embodiment 101. The library of multireporter cells any one of embodiments 05-100, wherein the multicistronic reporter vectors encode one or more transgenes that can be used to profile or distinguish a single or multiple biological pathways, cross-talk between two or more biological pathways, synthetic lethality, cellular homeostasis, organelle homeostasis, other cellular or subcellular phenotypes, cell-cell interactions, a toxicity response or other phenotypes and wherein expression of the transgene operably linked to the lineage-specific promoter is used to identify the cell type or the stage of differentiation.

Embodiment 102. The library of embodiment 101, wherein the biological pathway or phenotype is a pathway or phenotype associated with a disease.

Embodiment 103. The library of embodiment 102, wherein the disease is cancer, a cardiovascular disease a neurodegenerative or neurological disease or an autoimmune disease.

Embodiment 104. The library of embodiment 101, herein the biological pathway or phenotype is a pathway or phenotype associated with toxic response mechanism within the cell.

Embodiment 105. The library of embodiment 101, wherein the biological pathway or phenotype is a pathway or phenotype associated with aging.

Embodiment 106. The library of any one of embodiments 101-105, wherein the biological pathway is a pathway associated with cell proliferation, cell differentiation, cell survival, cell death, apoptosis, autophagy, DNA damage and repair, oxidative stress, chromatin/epigenetics, MAPK signaling, PI3K/Akt signaling, protein synthesis, translational control, protein degradation, cell cycle and checkpoint control, cellular metabolism, development and differentiation signaling, immunology and inflammation signaling, tyrosine kinase signaling, vesicle trafficking, cytoskeletal regulation or ubiquitin pathway.

Embodiment 107. The library of any one of embodiments 101-106, wherein the library comprises cells of two or more different lineages.

Embodiment 108. The library of embodiment 107, wherein the cells of different lineages comprise lineage-specific reporter polypeptides.

Embodiment 109. A kit comprising one or more multicistronic reporter vectors of any one of embodiments 1-39.

Embodiment 110. A kit comprising one or more multireporter stem cells of any one of embodiments 40-80.

Embodiment 11. The kit of embodiment 109 or 110, wherein the kit comprises a library of multicistronic reporter stem cells arrayed in a multiwell plate.

Embodiment 112. The kit of embodiment 111, wherein the stem cells in the multiwell plate are cryopreserved.

Embodiment 113. A method of profiling two or more polypeptides in a live cell, the method comprising determining the expression and/or location of the two or more of the transgenes of a multireporter stem cell of any one of embodiments 40-80.

Embodiment 114. The method of embodiment 113, wherein the profiling is performed before, during or after differentiation of the stem cell.

Embodiment 115. The method of embodiment 113 or 114, wherein the method is used to profile or distinguish a single or multiple biological pathways, cross-talk between two or more biological pathways, synthetic lethality, cellular homeostasis, organelle homeostasis, other cellular or subcellular phenotypes, cell-cell interactions or a toxicity response.

Embodiment 116. The method of any one of embodiments 113-115 wherein the expression and/or location of the two or more of the transgenes is determined at one or more time points.

Embodiment 117. The method of embodiment 116, wherein the expression and/or location of the two or more of the transgenes is determined at one or more of 1 minute, 5 minutes, 10 minutes, 15 minutes, 30 minutes, 45 minutes, 1 hour, 2 hours, 3 hour, 4 hours, 5 hours, 6 hours, 12 hours, 24 hours, 2 days, 4 days, 7 days, 14 days, 21 days, 30 days, 1 month, 3 month, 6 month, 9 month, 1 year, or more than 1 year.

Embodiment 118. A method of measuring the effects of an agent on the profile of two or mom polypeptides in a live cell, the method comprising subjecting a multireporter stem cell of any one of embodiments 40-77 to the agent and determining the expression and/or location of the two or more transgenes in the cell in response to the agent.

Embodiment 119. The method of embodiment 118, wherein the profiling is performed before, during or after differentiation of the stem cell.

Embodiment 120. The method of embodiment 118 or 119 wherein the agent is a drug or drug candidate.

Embodiment 121. The method ofany one of embodiments 118-120, wherein the agent is a cancer drug or cancer drug agent.

Embodiment 122. The method of any one of embodiments 118-121, wherein the method is a toxicology screen.

Embodiment 123. The method of any one of embodiments 118-122, wherein determining the expression and/or location of the two or more transgenes is performed in a library of multireporter cells.

Embodiment 124. The method of embodiment 123, wherein the lineage of cells in the library is determined by expression of the reporter polypeptide under the control of the lineage-specific reporter.

Embodiment 125. The method of any one of embodiments 118-124, wherein the profile is obtained using a single cell.

Embodiment 126. The method of embodiment 125, wherein the lineage of the single cell is determined by expression of the reporter polypeptide under the control of the lineage-specific reporter.

Embodiment 127. The method of any one of embodiments 118-126, wherein the expression and/or location of the two or more transgenes is measured by microscopy, high throughput microscopy, fluorescence-activated cell sorting (FACS), luminescence, using a plate reader, mass spectrometry, or deep sequencing.

Embodiment 128. The method of any one of embodiments 118-127, wherein cells of two or more different lineages are pooled to profile the two or more polypeptides in cells of two or more different lineages.

Embodiment 129. The method of embodiment 128, wherein the cells of different lineages comprise lineage-specific reporter polypeptides.

Embodiment 130. An acceptor cell for receiving a multicistronic reporter vector, wherein the acceptor cell comprises a recombinant nucleic acid integrated into a specific site in a host cell genome, wherein the recombinant nucleic acid comprises a first promoter operably linked to nucleic acid encoding a fusion polypeptide, wherein the fusion polypeptide comprises a reporter domain and a selectable marker domain, and wherein the nucleic acid comprises two site-specific recombinase nucleic acid sequence located at the 5′ end of the nucleic acid encoding the fusion polypeptide.

Embodiment 131. The acceptor cell of embodiment 130, wherein the nucleic acid composes two ATG sequences located 5′ to the two specific recombinase nucleic acid sequences.

Embodiment 132. The acceptor cell of embodiment 131, wherein the promoter is a constitutive promoter.

Embodiment 33. The acceptor cell of embodiment 133, wherein the constitutive promoter is a CMV promoter, a TK promoter, an eF1-alpha promoter, a Buck promoter, a PGK promoter, a CAG promoter, an SV40 promoter, or a human β-actin promoter.

Embodiment 134. The acceptor cell of any one of embodiments 130-133, wherein the site-specific recombinase sequence is a FRT nucleic acid sequence and/or an attP nucleic acid sequence and/or a loxP nucleic acid sequence.

Embodiment 133. The acceptor cell of embodiment 134, wherein the site-specific recombinase sequences comprise a PhiC31 attP nucleic acid sequence and a Bxb1 attP nucleic acid sequence.

Embodiment 136. The acceptor cell of any one of embodiments 130-135, wherein the reporter domain of the fusion polypeptide is a fluorescent reporter domain.

Embodiment 137. The acceptor cell of any one of embodiments 130-130, wherein the fluorescent reporter domain is selected from GFP, EGFP, Emerald, Citrine, Venus, mOrange, mCherry, TagBFP, mTurquoise, Cerulean, UnaG, dsRed, eqFP611, Dronpa, RFP, TagRFPs, TdTomato, KFP, EosFP, Dendra, IrisFP, iRFP and smURFP.

Embodiment 138. The acceptor cell of any one of embodiments 130-137, wherein the reporter domain of the fusion polypeptide is an mCherry reporter domain.

Embodiment 139. The acceptor cell of any one of embodiments 130-138, wherein the selectable marker domain of the fusion polypeptide confers resistance to hygromycin, Zeocin™, puromycin, blasticidin, neomycin or an analog of hygromycin, Zeocin™, puromycin, blasticidin, neomycin.

Embodiment 140. The acceptor cell of embodiment 139, wherein the promoter is a human β-actin promoter or a CAG promoter.

Embodiment 141. The acceptor cell of an one of embodiments 130-140, wherein the recombinant nucleic acid is integrated in an adeno-associated virus S1 (AAVS1) locus, a chemokine (CC motif receptor 5 (CCR5) locus, a human ortholog of the mouse ROSA26 locus, a hip11 (H11) locus or the citrate like beta like gene locus (CLYBL).

Embodiment 142. The acceptor cell of any one of embodiments 130-141, wherein the cell is a pluripotent cell an induced pluripotent stem cell, or a multipotent cell.

Embodiment 143. The acceptor cell of embodiment 142, wherein the induced pluripotent stem cell is a WTC-11 cell or a NCRM5 cell.

Embodiment 144. The acceptor cell of any one of embodiments 130-142, wherein die cell is a primary cell.

Embodiment 145. The acceptor cell of any one of embodiments 130-142, wherein the cell is an immortalized cell.

Embodiment 146. The acceptor cell of embodiment 145, wherein the immortalized cell is a HEK293T cell, an A549 cell, an U2OS cell, an RPE cell, an NPC1 cell, a MC7 cell, a HepG2 cell, a HaCat cell, a TK6 cell, an A375 cell or a HeLa cell.

Embodiment 147. The acceptor cell of any one of embodiments 130-146, wherein the acceptor cell comprises a first recombinant nucleic acid for receiving a first multicistronic reporter vector and a second recombinant nucleic acid for receiving a second expression construct, wherein the first recombinant nucleic acid is integrated into a first specific site in a host cell genome and the second recombinant nucleic acid is integrated into a second specific site in a host cell genome.

Embodiment 148. The acceptor cell of embodiment 147, wherein the second recombinant nucleic acid encodes a polypeptide, a reporter polypeptide, a cykitoxic polypeptide, a selectable polypeptide, a constitutive Cas9 expression vector or inducible Cas9 expression vector.

Embodiment 149. A reporter cell prepared from the acceptor cell of embodiment 137 or 148, wherein a multicistronic reporter vector is integrated into the first specific site and a constitutive or inducible Cas9 expression vector is integrated into a second specific site.

Embodiment 150. A method wherein a reporter cell of embodiment 149 is arrayed in a multiwell plate and used as the basis for a screen using single or oligo pool sgRNAs.

Embodiment 151. A method for generating an acceptor cell for receiving a multicistronic reporter vector, the method comprising introducing a recombinant nucleic acid to a cell wherein the recombinant nucleic acid comprising 5′ to 3′ a first nucleic acid for targeting homologous recombination to a specific site in the cell, a first promoter, two ATG sequences, two site-specific recombinase nucleic acid, nucleic acid encoding a first reporter polypeptide and a selectable marker, a second nucleic acid for targeting homologous recombination to a specific site in the cell, a second promoter and nucleic acid encoding a second reporter polypeptide or a cytotoxic polypeptide,
wherein expression of the first reporter polypeptide without expression of the second reporter polypeptide or cytotoxic polypeptide indicates targeting integration of the recombinant nucleic acid to the specific site in the cellular genome and expression of the first and second reporter or cytotoxic polypeptides indicates random integration in the cellular genome.

Embodiment 152. The method of embodiment 151 wherein the recombinant nucleic acid is integrated into the genome of the cell using:
an RNA guided recombination system comprising a nuclease and a guide RNA
a TALEN endonuclease, or
a ZFN endonuclease.

Embodiment 153. The method of embodiment 151 or 152, wherein cells expressing the First reporter polypeptide but not expressing the second reporter polypeptide are selected.

Embodiment 154. The method of any one of embodiments 151-153, wherein the site-specific recombinase nucleic acids comprise a FRT nucleic acid sequence and/or an attP nucleic acid sequence and/or a loxP nucleic acid sequence.

Embodiment 155. The method of any one of embodiments 151-154, wherein the first reporter polypeptide is fluorescent polypeptide and the second reporter polypeptide is a different fluorescent polypeptide.

Embodiment 156. The method of any one of embodiments 151-155, wherein the first and second reporter polypeptide is selected from GFP, EGFP, Emerald, Citrine, Venus, mOrange, mCherry. TagBFP, mTurquoise, Cerulean, UnaG, dsRed, eqFP611, Dronpa, RFP, TagRFPs, TdTomato, KFP, EosFP, Dendra, IrisFP, iRFP and smURFP.

Embodiment 157. The method of embodiment 156, wherein the first reporter polypeptide is an mCherry reporter and the second reporter polypeptide is GFP.

Embodiment 158. The method of any one of embodiments 151-155, wherein the cytotoxic polypeptide is a thymidine kinase peptide or a diphtheria toxin A (DTA).

Embodiment 159. The method of any one of embodiments 151-158, wherein the selectable marker confers resistance to hygromycin, Zeocin™, puromycin, blasticidin, neomycin or an analog of hygromycin, Zeocin™, puromycin, blasticidin, neomycin.

Embodiment 160. The method of any one of embodiments 151-159, wherein the first promoter is a CMV promoter, a TK promoter, an eF1-alpha promoter, a UbC promoter, a PGK promoter, a CAG promoter, an SV40 promoter, or a human β-actin promoter and the second promoter is a CMV promoter, a TK promoter, an eF1-alpha promoter, a UbC promoter, a PGK promoter, a CAG promoter, an SV40 promoter, or a human β-actin promoter.

Embodiment 161. The method of any one of embodiments 151-160, wherein the first nucleic acid for targeting homologous recombination and the second nucleic acid for targeting homologous recombination target recombination to an AAVS1 locus, a CCR5 locus, a human ortholog of the mouse ROSA26 locus, a H11 locus or a CLYBL locus.

Embodiment 162. The method of any one of embodiments 151-161, wherein the cell is an immortalized cell.

Embodiment 163. The method of embodiment 162, wherein the immortalized cell is a HEK293T cell, an A549 cell, an U2OS cell, an RPE cell, an NPC1 cell, a MCF7 cell, a HepG2 cell, a HaCat cell, a TK6 cell, an A375 cell or a HeLa cell.

Embodiment 164. The method of any one of embodiments 151-163, wherein the cell is a pluripotent cell, an induced pluripotent stem cell, or a multipotent cell.

Embodiment 165. The method of embodiment 164, wherein the induced pluripotent stem cell is a WTC-11 cell or a NCRM5 cell.

Embodiment 166. The method of any one of embodiments 151-161, wherein the cell is a primary cell.

Embodiment 167. The method of any one of embodiments 151-166, further comprising introducing a second recombinant nucleic acid to a cell for receiving a second multicistronic reporter vector wherein the second recombinant nucleic acid comprises 5′ to 3′ a third nucleic acid for targeting homologous recombination to a specific site in the cell, a third promoter, two ATG sequences, two site-specific recombinase nucleic acid, nucleic acid encoding a third reporter polypeptide and a selectable marker, a fourth nucleic acid for targeting homologous recombination to a specific site in the cell, a fourth promoter and nucleic acid encoding a fourth reporter polypeptide or cytotoxic polypeptide,
wherein expression of the third reporter polypeptide without expression of the fourth reporter or cytotoxic polypeptide indicates targeting integration of the recombinant nucleic acid to the specific site in the cellular genome and expression of the third and fourth reporter or cytotoxic polypeptides indicates random integration in the cellular genome.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further details of the invention am illustrated by the following non-limiting Examples. The disclosures of all references in the specification are expressly incorporated herein by reference.

### EXAMPLES

### Example 1—Generation of a Single Acceptor Site Lineage Barcode Model

A robust targeting strategy using RNA-guided genome engineering tools mediated by Cas9 to introduce an ‘acceptor site’ into the endogenous AAVS1 locus of iPSC lines was generated as previonsly described (PCT/US2018/032834). The acceptor site includes (1) an mCherry fluorescence marker to confirm acceptor site integration; (2) an antibiotic resistance gene driven by the cytomegalovirus/chicken β-Actin promoter (CAG) promoter to enable ccli selection; (3) a TetR element driven by a constitutive CAG promoter for optional inducible expression of genes; and (4) a GFP gene driven by the CMV promoter localized downstream of the homologous-recombination region to enable prompt distinction between random and targeted integrations (cells with random integration would fluoresce green due to GFP expression, while cells with targeted integration would not due to loss of CMV-GFP).

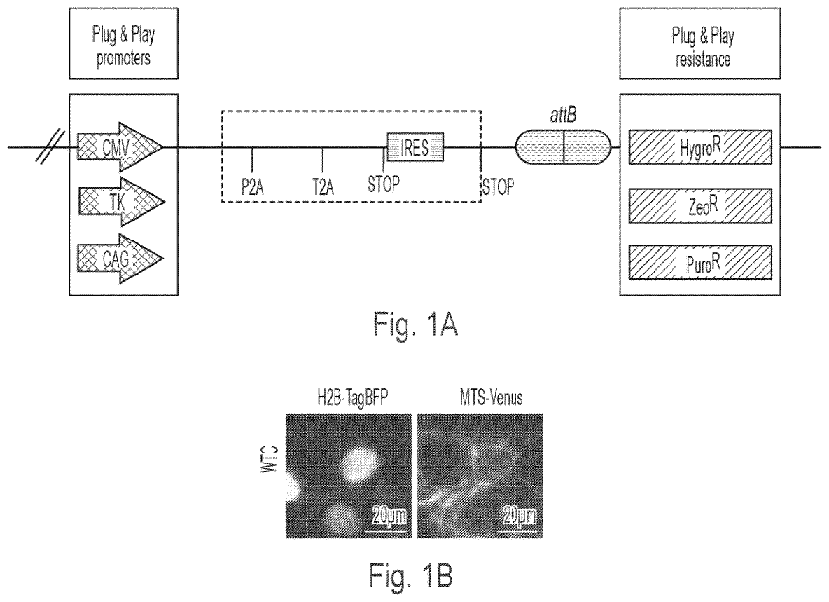

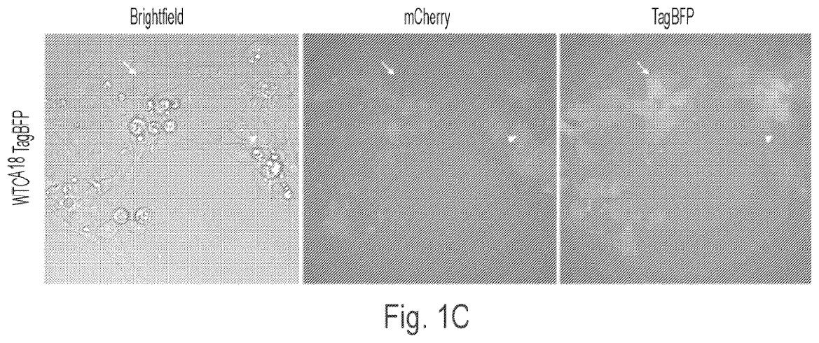

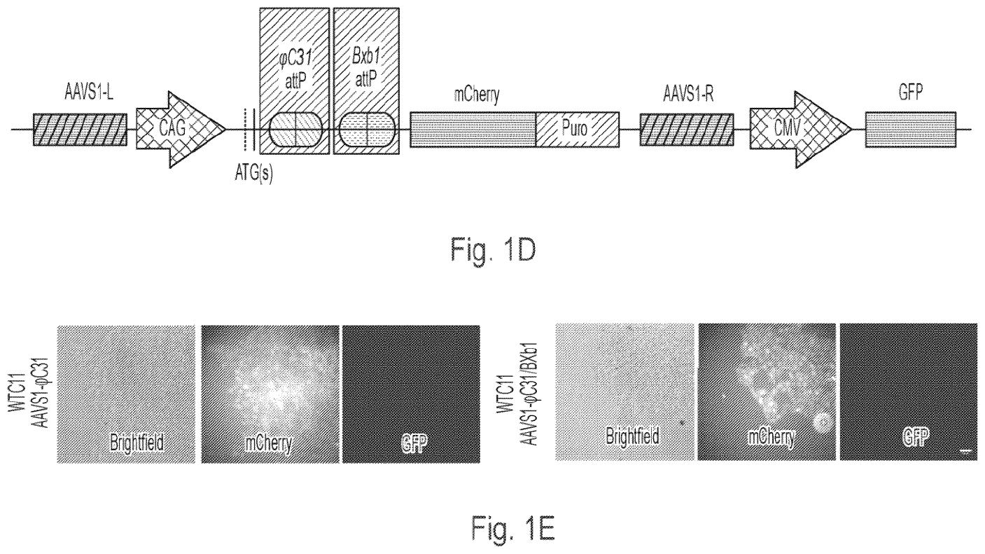

An improved tetracistronic reporter was generated to integrate up to 4 genes in the acceptor site. The tetracistronic reporter contains a constitutive promoter that drives the expression of a single open reading frame (ORF) containing three multiple cloning sites (MCSs) separated by 2 unique viral 2A self-cleaving peptides and a fourth MCS separated by an internal ribosome entry site (IRES) element that allows for translation initiation in the middle of the mRNA sequence (FIG. 1A). The 2A self-cleaving peptides allow multiple proteins to be encoded as polyproteins, which dissociate into component proteins upon translation. The 2A peptide sequence impairs normal peptide bond formation through a mechanism of ribosomal skipping. From the family of peptide 2A cleavage sequences the P2A and T2A versions were identified as optimal candidates since these have been shown to exhibit most efficient cleavage (Kim J H, et al., PLoS ONE 6(4): e18556 (2011)). To increase cleavage efficiency of the viral peptide, a Gly-Ser-Gly linker was added between the protein N-terminal and the 2A-peptide sequences. Furthermore, the attB sequence, specific for Bxb1 recombinases, was fused to a promoterless resistance gene that can only be expressed when correct gene targeting occurs into the acceptor site. Both the promoter and the resistance are plug-and-play and can be swapped for desired configuration of the multicistronic vector.

Reporter localization in WTC iPSC cells was tested by transiently transfecting IPSCs having a plug-and-play AASV1 acceptor site with a multicistronic vector containing H2B fused to TagBFP and mitochondrial targeting sequence (MTS) fused to Venus (FIG. 1A). The localization was assessed by microscopy (FIG. 1B), demonstrating that the proteins expressed from the multicistronic vector are functional.

Another multicistronic vector carrying a TagBFP was used to test the recombination in iPSCs. This tester-vector was co-transfected with a vector expressing Bxb1 recombinase into acceptor cell lines and transfection conditions were optimized for production of stable reporter cell lines that may be used as a basis for assay development and disease modeling. The recombination rate of the Bxb1 recombinase employed is of one in 10⁻¹ in HEK293 cells (Duportet, X. et al NAS 42(21), 13440-13451 (2014). Loss of cytoplasmic mCherry fluorescence in cells expressing TagBFP confirms stable recombination of the control reporter at the acceptor site while cells that do not express TagBFP retain cytoplasmic mCherry expression and are not recombined (FIG. 1C).

The acceptor site was further optimized to be smaller and more adaptable. As illustrated in FIG. 1D, the updated acceptor site includes 1) one extra serine recombinase site for φC31 which allows for testing the efficiencies of the 2 different recombinase; 2) two alternative ATGs placed in different reading frames to allow for expression of the downstream fluorophore fused to resistance marker post-recombination in the attP site, 3) the resistance marker fused to mCherry is puromycin since puromycin has been shown to kill cells quicker than Zeocin. Furthermore, the removal of the TetR element and its promoter reduce the size of the acceptor site by 33% allowing for a higher yield of recombined cells.

The optimized acceptor site was stably integrated into an integration site within the genome of WTC and NCRM5 hiPSC using established Cas9-mediated genome editing protocols (FIC. 1E). As for the acceptor site design, the safe harbor AAVS1 locus was used as the integration site in accordance with design of the acceptor site. The acceptor site was synthesized aid cloned into an AAVS1-Donor vector (GeneCopoeia) and co-transfected with Cas9:sgRNA(AAVS1), a guide RNA (gRNA), complementary to the AAVS1 sequence of interest, resulting in stable integration at a single integration site, namely the AAVS1 locus located between exons 1 and 2 of the PPP1R12C locus of chromosome 19. Transfection and stable integration were done using a RNA-guided Cas9-CRISPR-mediated genome editing followed by antibiotic selection with Puromycin. After clonal cell growth, 2 junction PCR were used to identify single allele integration of the acceptor site. A second pair of primers amplifies a PCR product from alleles where integration occurred. Clonal cells with single allele integration should present amplification for both PCRs. Next, to determine copy number integration at the correct site droplet digital PCR (ddPCR) was conducted with 2 specific probes for mCherry and puromycin genes, only clones with 0.8<CNV<1.4 for both genes were considered positive. A probe for the housekeeping gene RPP30 with 2 copies in the human genome, was used to quantify the relative copy number in the samples (Table 1).

TABLE 1

| | | N clones | | N clones | % of clones |
|---|---|---|---|---|---|
| | | integration | N clones | with 1 copy | with single- |
| | | at the | single | (ddPCR) | copy at the |
| | N clones | AAVS1 | allele | (mCherry & | AAVS1 |
| | screened | locus | integration | Puromycin) | locus |
| WTC- | 33 | 14 | 14 | 1 | 3% |
| PhiC31 | | | | | |
| WTC- | 24 | 24 | 24 | 0 | 0% |
| Bxb1 | | | | | |
| WTC- | 33 | 10 | 10 | 1 | 3% |
| PhiC31/ | | | | | |
| Bxb1 | | | | | |
| NCRM5- | 30 | 1 | 1 | 0 | 0% |
| PhiC31 | | | | | |
| NCRM5- | 4 | 4 | 4 | 3 | 75% |
| Bxb1 | | | | | |
| NCRM5- | 44 | 6 | 5 | 1 | 2% |
| PhiC31/ | | | | | |
| Bxb1 | | | | | |

### Example 2—Multicolor iPSC-Derived Cardiomyocyte Cells with Lineage-Specific Fluorescent Reporter

### Generation of PSC-Derived CM-Lineage Barcoded Cells

To expand the iPSC line specificity, iPSC acceptor lines were engineered with vectors including tissue specific promoters for various cardiovascular cell lineages.

To expand the iPSC cardiac line specificity and applications, a set of 3 lineage-specific reporters were engineered to differentiate atrial, ventricular and nodal cardiomyocyte (CM) lineages (FIG. 2A). To achieve CM-lineage barcoding, promoters that are highly specific for each of the CM lineages were chosen (Table 2). Myosin light chain 2v (MCL2v) is expressed exclusively in the ventricles (Chen et al., J Biol Chem. 273(2), 1252-6 (1998)), where it contributes to the formation of sarcomeres and increases Ca²⁺ sensitivity at submaximal Ca²⁺ concentrations (Chen, Z. et al., Eur Heart J 38(4) 292-301 (2016)). The expression of sarcolipin (SLN), an inhibitor of the sarcoplasmic reticulum Ca²⁺-ATPase, is restricted to the atrial lineage in the developing heart of mammals including humans (Minamisawa et al., J Biol Chem. 278(11):9570-5 (2003), Babu et al., J Mol Cell Cardiol. 43(2):215-22 (2007)). Furthermore, SLN expression can be used as a marker to monitor and isolate hiPSC-derived atrial-like myocytes and it has been shown to be more abundant in atrial like cells and undetectable in ventricular ones (Minamisawa et al., J Biol Chem. 278(11):9570-5 (2003); Josowitz et al., PLoS One, 9(7):e101316 (2014)). The short stature homeobox 2 (SHOX2) promoter has been shown to have its expression restricted to nodal cells of the heart (Espinoza-Lewin et al., Dev Biol. 327(2): 376-385 (2009)), and was used to visualize nodal-like cells.

TABLE 2

| Cardio | | |
|---|---|---|
| Cell | | |
| Lineage | Promoter | Promoter Details |
| Ventricular | MLC2v | 600 bp MLC2v enhancer |
| | (myosin light chain 2v) | |
| Atrial | SLN | ~3.5 kb promoter element |
| | (sarcolipin promoter) | preceding the SLN |
| | | transcription start site |
| Nodal | SHOX2 | ~3.5 kb promoter element |
| | (short stature homeobox | preceding the SHOX2 |
| | 2 promoter) | transcription start site |

For functional evaluation of CM lineages, each vector was driven by a lineage specific promoter and carried a H2B unique fluorophore as a lineage barcode (FIG. 2B). For structural evaluation of CM lineages, each vector carried the lineage barcode (H2-fluorophore) and actinin and mitochondria reporters.

Three vector design versions were developed: (1) CM-1-vectors where functional or structural reporters are driven by CM lineage specific promoters, the smallest variation of the structural vectors. (2) CM-2-vectors where the CM lineage specific promoter drives the Tet^{OFF} system, activating expression of both the functional or structural reporters, allowing increased promoter activity, as the Tet^{OFF} system amplifies the lineage specific promoter, and (3) CM-3-vectors where the barcode is driven by CM lineage specific promoters and a constitutive promoter drives expression of the functional or structural reporters. This vector bypasses potential issues of inadequate promoter strength for downstream reporter expression through inclusion of an additional transcript unit that reduces the number of genes driven by the CM-specific promoter (FIG. 2B).

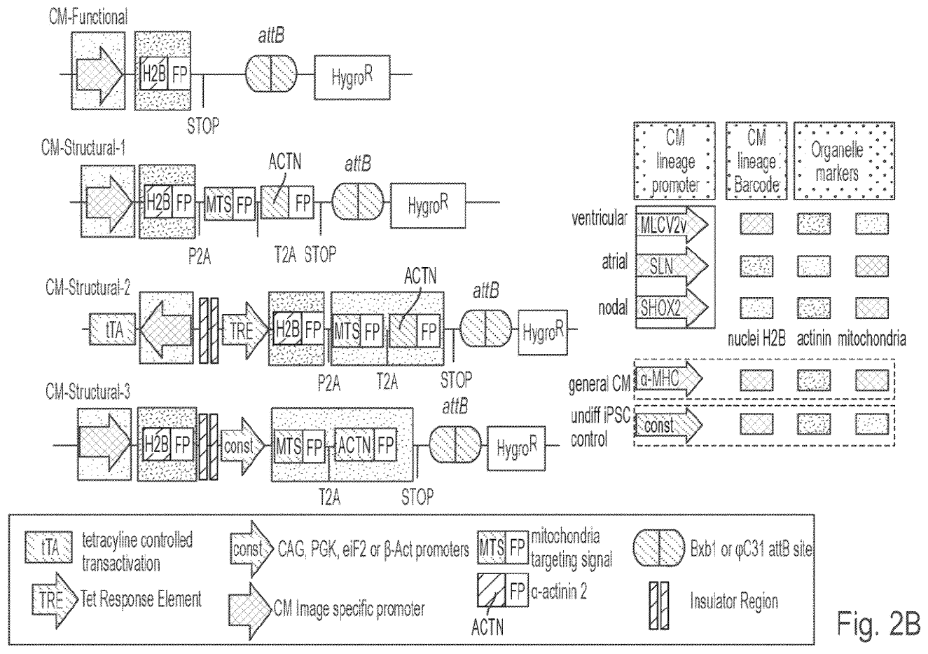

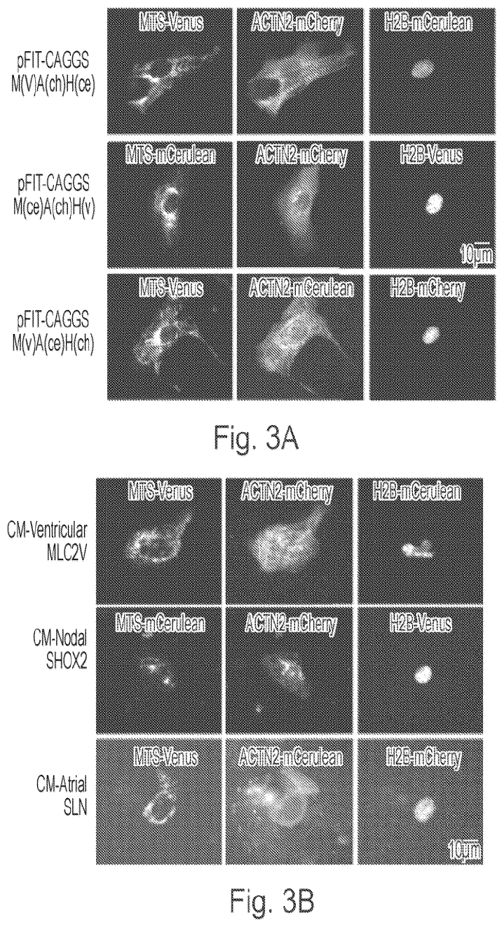

The CM-Structural-1 vectors driven by the constitutive promoter CAG (FIG. 3A) or by each of the lineage specific promoter (FIG. 38) are transfected in hiPSC-derived CM and the expression and localization of each of the markers: nuclei (H2B), mitochondria (MTS) and a-actinin, is observed.

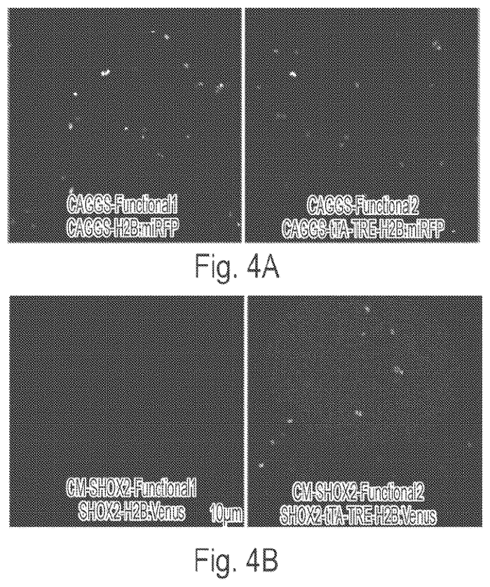

The CM-Functional-2 driven by the Tet^{OFF} system to amplify the lineage specific promoters system leads to an increase in the number of hiPSC-derived CM cells expressing the reporter vector (FIG. 4B), while the same system driven by CAG constitutive promoter does not lead to a significant difference in the number of expressed cells (FIG. 4A).

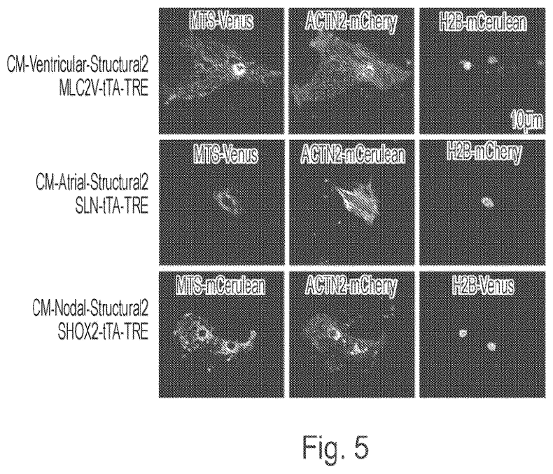

The CM-Structural-2 reporters driven by CM-lineage specific promoters (MLC2v, SHOX2, SLN2) under the transcriptional control of tTA-TRE lead to expression of the 3 markers: nuclei (H2B), mitochondria (MTS) and a-actinin (FIG. 5). Using this system there is an enhancement of the expression levels and number of expressing cells compared to the same reporter without the tTA-TRE.

Differentiation of iPSC-Derived CM-Lineage Barcoded Cells

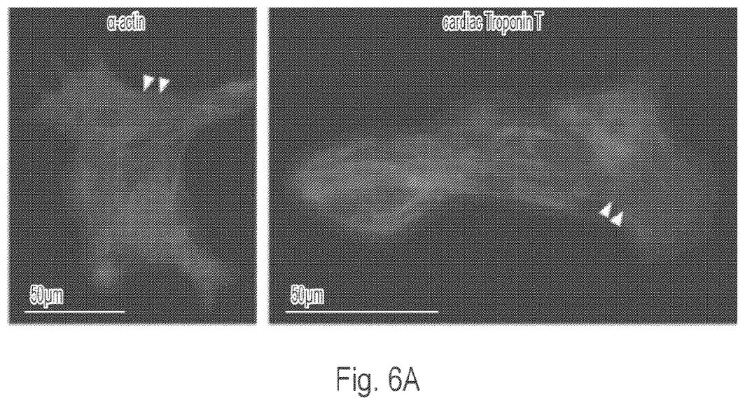

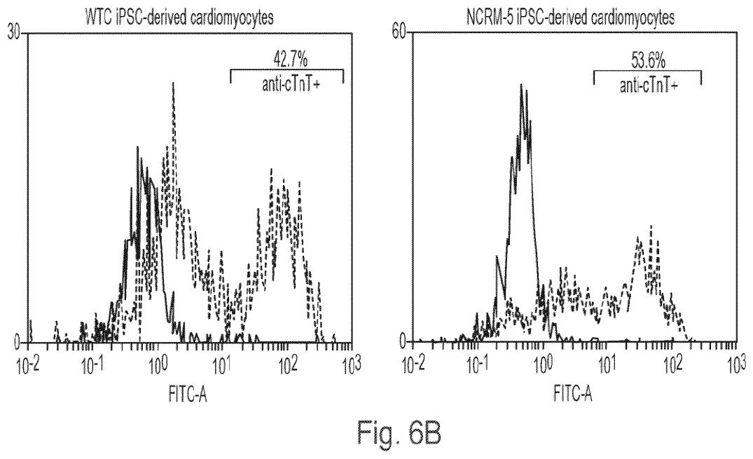

An adapted version of the standard protocol based on temporal modulation of the canonical Wnt signaling was used to direct iPSC CM differentiation (Lian X, Proc. Natl. Acad. Sci. U.S.A. July 3; 109(27) (2012)). The differentiated cells spontaneously beat and aggregated, and imaging of immunofluorescence showed expression of cardiomyocyte-associated proteins α-actinin and cardiac Troponin T and beating phenotype. (FIG. 6A). Further quantification of the percentage of iPSC-derived cardiomyocytes in cultures, using flow cytometry, showed that WTC and NCRM-5 iPSC line preparations contained 42.7% and 53.6% cells, respectively, with positive labeling for a cardiac marker (Troponin T) (FIG. 6B). Further, using a cardiomyocyte purification method based on biochemical differences in glucose and lactate metabolism between cardiomyocytes and non-cardiomyocytes, an increase in the percentage of cardiomyocytes recovered was achieved. This approach resulted in recovery of >75% cardiomyocytes. The differentiated culture comprises different ratios of the 3 CM lineages.

To confirm barcode lineage identity, immunofluorescent staining of endogenous lineage-specific markers and flow cytometry are used to carry out correlation analyses between the promoter-driven barcode expression and expression of endogenous markers for: 1) the corresponding lineage (e.g. compare vehicular barcode expression and immunolabeling of endogenous ventricular MCL2 isoform); and 2) other lineages (e.g. compare ventricular fluorescent barcode expression with immunolabeling of endogenous MCL2a and HCN4 channel proteins, which are specific for atrial and nodal-like cells, respectively). The performance of each fluorescent barcode is assessed by quantifying: barcode efficiency—proportion of lineage barcoded cells that are correctly immunolabeled for endogenous markers of the corresponding lineage; barcode specificity—proportion of lineage barcoded cells that do not express endogenous markers for the other lineages; and barcode accuracy—proportion of lineage barcoded cells that are correctly identified by the corresponding endogenous immunolabeling and do not express endogenous markers for the other cell lineages.

Use of iPSC-Derived CM-Lineage Barcoded Cells in Functional Assays

Cardiotoxicity assays probe the cardiac liability of compounds earlier in the drug development process. The cardiotoxicity assay incorporates both functional toxicity (alterations of the mechanical function of cardiomyocytes) and structural toxicity (morphological damages to cardiomyocytes, changes to intracellular organelles and loss of cardiomyocyte viability), providing a full and efficient assessment of cardiac liabilities of new and existing chemical entities. The cardiotoxicity assay has both the ability to detect cardiotoxic liabilities that are detected by industry-standard preclinical assays (true positives) and the ability to detect cardiotoxic liabilities that were identified in the clinic but were not detected by existing preclinical assays (false negatives). More importantly, the ability to determine the lineage specific cardiotoxicity will greatly improve the power of the assay by allowing a lineage fingerprinting.

Demonstrating these capabilities will deliver an efficient, cost-effective in vitro screening tool to the preclinical cardiac safety assessment community that will be complementary to the widely accepted electrophysiology and multielectrode array approaches currently used in the field. The cardiotoxicity assay greatly improves the ability of preclinical safety testing to predict clinical cardiotoxicity, allowing for widespread adoption of this assay in the pharmaceutical industry.

The assay is used to detect cardiotoxicities that are detected by industry-standard preclinical assays, using compounds with known cardiotoxicity, including ion channel blockage, mitochondrial toxicity, arrhythmia, fibrosis, and many more. For example, the SCREEN-WELL® Cardiotoxicity Library from Enzo, which is a 134) compound reference library for cardiotoxicity studies with a variety of structurally and mechanistically different compound classes, as well as nontoxic controls, can be used in the assay. Use of thus library in the assay demonstrates that the assay detects known cardiotoxicities.

The iPSC-derived, barcoded CM lineage cells can further be used to predict clinical cardiotoxicities that are not predicted by industry-standard preclinical assays, using compounds that have shown clinical cardiac safety signals that were not identified in preclinical testing, e.g. COX-2 inhibitor rofecoxib (Vioxx) used for the treatment of inflammatory conditions (in 2004) and a serotonin 4 receptor agonist, teagaserod (Zelnorm/Zelmac), used in irritable bowel syndrome (in 2007) This subset of compounds demonstrates the sensitivity and predictive power of the assay.

The assay can similarly be used to detect cardiotoxicities of combination therapies. i.e. cytotoxic agents and targeted therapies used to treat cancer. The targeted oncology therapies in development are tested for toxicity in isolation (Hasinoff et al., Toxicol Appl. Pharmacol. 249, 132-139 (2010): Force et al., Nat. Rev. Drug Discov. 10, 111-126 (2011)), consequently there is a need for new and predictive preclinical approaches to screen combination therapies. A panel of chemotherapeutic compounds (e.g. anthracycline-like chemotherapeutics such as doxorubicin, daunorubicin and epirubicin) and targeted compounds (e.g. Trastuzumab, a monoclonal antibody against HER2), are tested separately aid in combination in the assay to reveal increased toxicity associated with the combination therapy.

To conduct the assay, the 3 CM lineages are pooled together and plated in a 34 well plate. The image acquisition for the structural assay comprises the acquisition of 4 channels (3 fluorescent channels and phase contrast) over 24 h. The assay is run in parallel. In undifferentiated reporter hiPSC that express the same organelle markers as their counterpart CM-lineage specific reporter cells. This allows differentiation of general cytoxicity from cardiotoxicity and further allows the detection of any possible phototoxicity caused by endogenous expression of fluorescently labeled proteins. The data collected is divided into labeled—when the mechanism of toxicity is known to affect the relevant phenotypic markers; and unlabelled—when the mechanism of toxicity have not been associated with any phenotypic marker alteration.

Many structural and functional readouts are evaluated during and/or after the cardiotoxicity assay. Exemplary readouts are summarized in Table 3.

TABLE 3

| | | Organelle/ | |
|---|---|---|---|
| | | reporter | |
| Assay | Reporter | segmentation | Features |
| Structural | H2B-FP | Nuclei | CM cell lineage |
| | | | Cell count/lineage |
| | | | Cell viability |
| | | | Nuclei area |
| | MTS-FP | Mitochondria | Mitochondria area |
| | | | Mitochondria morphology |
| | ACTN2-FP | Sarcomere | Sarcomeric structure pattern |
| | | | Alpha-actinin levels |
| | | | Cell size |
| Functional | Data driven image analysis | | Beating: |
| | | | Frequency |
| | | | Period |
| | | | Duration |
| | | | Amplitude |
| | | | variation |

The resulting structural properties of CM cells are evaluated. Exemplary properties include morphological damage of cardiomyocytes, changes to intracellular organelles, or loss of cardiomyocyte viability. Compounds that are known to perturb cardiomyocyte structure, e.g. Doxorubicin (apoptosis induction in cardiomyocytes (Minotti et al., Pharmacol. Rev. 56, 185-229 (2004))), Sunitinib (mitochondrial toxicity (Cho et al., Lancet 370, 2011-2019 (2007) and amiodarone (mitochondrial toxicity (Deres et al. J. Cardiovasc. Pharmacol 45, 36-43 (2005))) are used as positive controls.

The resulting functional properties of CM cells are also evaluated. Exemplary properties include alterations in synchronous rhythmic beating as a phenotype to evaluate the cardiac-specific function, Compounds that are known to perturb cardiomyocyte function, such as arrhythmias, e.g. Cisapride (hERG inhibitor), Nifedipine (Ca²⁺ inhibitor) and Quinidine (Na⁺/Ca²⁺, hERG inhibitor) are used as positive controls. The specificity and sensitivity of the beating is analyzed and can be compared to published results from other methods such as patch-clamp, microelectrode arrays (MEA) or cellular impedance.

The functional properties of cells are evaluated using fast image data acquisition of cardiomyocyte contractility using videomicroscopy. Motion analysis on the image sequence is used to capture and quantify the biomechanical beating of cardiomyocytes by identifying changes in the image intensity due to cardiomyocyte contraction and relaxation. The algorithm design is guided by the fact that it is possible to work on different tissue types without the need for any parameter tuning, and a data-driven approach avoids the use of specific cell segmentation algorithms. An exemplary beating analysis pipeline consists of a series of steps such as: (1) block-wise segmentation of the image sequence, (2) extraction of the heating signal based on signal correlation, (3) quantification of the beating signals, (4) outlier removal, and and (5) clustering of the beating signals based on fluorescent lineage barcoding (Maddah et al., Stem cell reports 4, 621-31 (2015)). Cell beating detection is performed on a microscope with exemplary features such as: a high-quality stage-top incubator that keeps the temperature and CO levels uniform, a high-speed CMOS camera for fast image capture, and a high-precision motorized xy stage, which enables scanning of standard multi-well plates (such as 96- and 384-well). Images are captured by the camera at a rate of up to 30 frames/s at 2048×2048 pixel resolution (Maddah et al., Stem cell reports 4, 621-31 (2015)). Software analysis allows for quantification of beating parameters such as frequency, period, duration, amplitude and variation.

Video analysis software is used for automated extraction of beating parameters upon exposing hiPSC-CM lineage reporter cells to known compounds with effects in beating frequency and periodicity ((e.g. Cisapride (hERG inhibitor), Nifedipine (Ca²⁺ inhibitor) and Quinidine (Na⁺/Ca²⁺, hERG inhibitor)). The image acquisition is carried out with a 10× phase contrast objective followed by image acquisition of 3 fluorescent channels. As a control, the assay is run in parallel in the acceptor hiPSC. This enables the detection of any possible alteration caused by engineering the reporter cells. The data collected will be divided into labelled—when the Mechanism of toxicity is known to affect the beating parameters; and unlabelled—when the mechanism of toxicity has not previously been associated with any beating alterations.

### Example 3—Single Acceptor Site with Neuro-Tox Constructs

Generation of iPSC-Derived Neural-Lineage Barcoded Cells

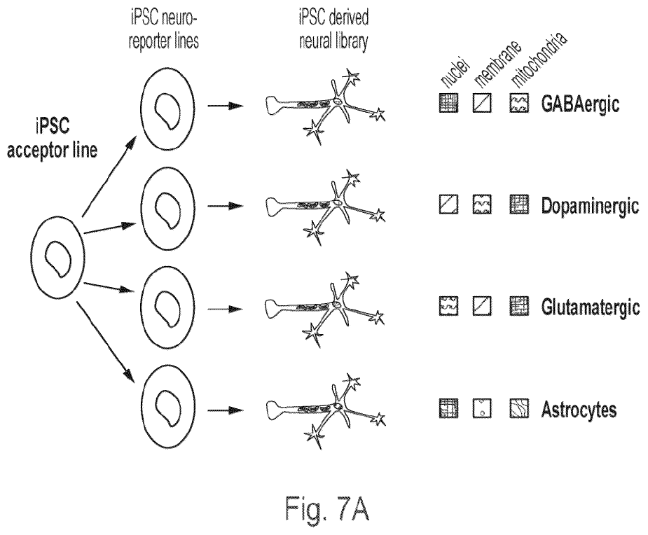

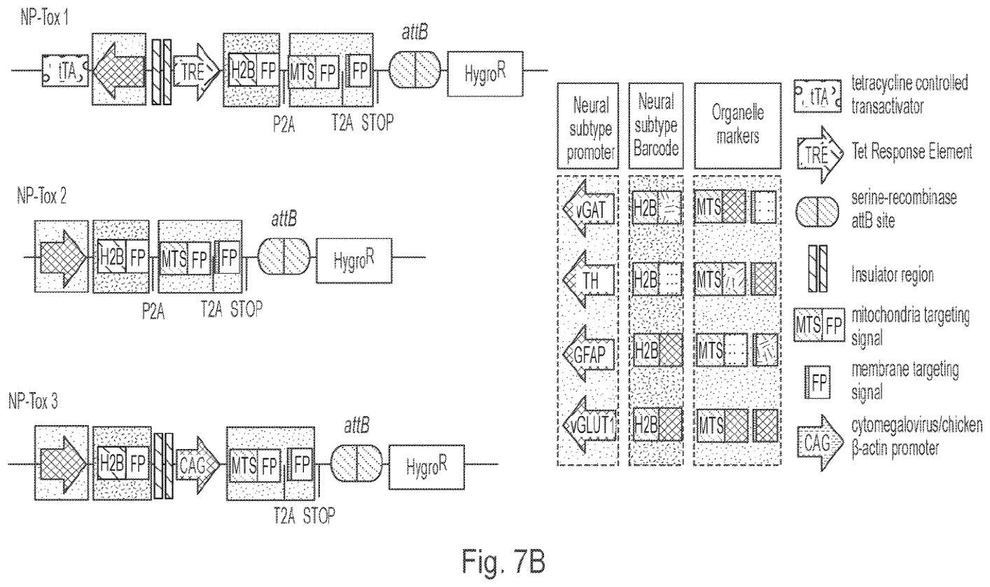

iPSC acceptor lines were engineered with vectors including tissue specific promoters for various neuronal cell lineages. A set of 4 lineage-specific reporters were engineered to differentiate neuronal cells into the following lineages: GABAergic, Dopaminergic. Glutamatergic and astrocytes (FIG. 7A). To achieve neural lineage specificity 4 promoters were selected based on availability of smaller truncations or chimeras with demonstrated lineage specificity to allow mitigation of potential difficulties associated with assembly of large vector constructs (FIG. 7B).

The neural lineage specific vectors are driven by a lineage specific promoter and carry a H2B unique fluorophore that works as a lineage barcode (FIG. 7B). The vector design further includes spectrally distinct fluorescent protein fused to reporters that enable visualization of the mitochondria and plasma membrane in hiPSC-derived neural cells. Three exemplary strategies for generating functional neural lineage-specific vectors are: (1) NP-Tox1, which delivers enhanced promoter activity, circumventing potential low neural-specific promoter expression levels, through a Tet^{OFF} system, which showed increased transduction efficiency of GFP compared with the human synapsin promoter when implemented in rat immortalized neuronal cell lines (Alexopoulou et al. BMC Cell Biol. 9, 2 (2008)); (2) NP-Tox2, which addresses issues of reduced recombination efficiency with increased vector size; and (3) NP-Tox3, which bypasses potential issues of inadequate promoter strength for downstream reporter expression by educing the number of genes driven by the neural specific promoter.

Differentiation of iPSC-Derived Neural-Lineage Barcoded Cells

Standard protocols are used to differentiate iPSCs to neural lineages. For example, differentiation protocols based on a common progenitor that give rise to both neurons and astrocytes (Shi Y et al. Nat. Protoc., 7(10):1836-46 (2012)). Dopaminergic, Gabaergic and glutamatergic neuronal cells are generated using established protocols (See, e.g., Hong et al., J Neurochem, 104(2): 316-324 (2008); Maroof et al., Cell Stem Cell, 12(5):55-72 (2013); and Yap et al. Stem Cells Int. 2015). Barcode lineage identity is confirmed using immunofluorescence and flow cytometry to carry-out correlation analyses between the promoter-driven barcode expression and expression of endogenous markers for (1) the corresponding lineage (e.g. compare dopaminergic barcode expression and immunolabeling of endogenous TH protein); and (2) other lineages (e.g. compare dopaminergic fluorescent barcode expression with immunolabeling of endogenous vGAT, vGluT and CD44 proteins, which are specific for GABAergic neurons, glutamatergic neurons, and astrocytes, respectively). Using these analyses, the following parameters are quantified: barcode accuracy—percentage of lineage barcoded cells that express the corresponding endogenous lineage-specific markers, barcode efficiency—percentage of cells that express lineage-specific endogenous markers and also express the corresponding lineage barcode, and barcode specificity-percentage of lineage barcoded cells that do not express endogenous markers of other cell lineages.

Use of iPSC-Derived Neural-Lineage Barcoded Cells in Functional Assays

Neurotoxicity assays ae Screening assays that have the potential to characterize the activity of compounds that might adversely affect the nervous system. At in vitro, high-throughput neurotoxicity quantitative assay, based on the phenotypic profiling of iPSC-derived neural cells, is used to determine effective toxic concentrations of compounds and to compart the effects of different compounds. The neurotoxicity assay is not limited to testing candidate pharmaceuticals, it also allows testing of any compound with unknown neurotoxicity, including environmental agents, pesticides, cosmetics, food additives, and dietary supplements Many of these types of compounds are rarely tested, and the assay provides both a robust in vitro assay for assessment of their safety and enables testing of combinations of compounds and drugs that may not elicit a response when examined in isolation. Due to the increasing prevalence of neurological disorders and the large number of untested compounds, there is a pressing used to develop an efficient and reliable tool to identify neurotoxicants.

The neurotoxicity assay is validated using a collection of compounds (Table 4) a) readout-specific assessment set—this set of compounds is used to evaluate the technical performance of readout measurements, characterize the readout response, and establish the dynamic response range, and contains compounds with a robust effect on the targeted readout; b) training set—this set of compounds is used to (1) to detect neurotoxicities that have been previously identified by assays that use neurite outgrowth as a readout; (2) to discriminate between general toxic responses and specific neurotoxic effects by comparing the readout responses of neural versus non-neural cells (undifferentiated reporter hiPSC) to the same set of chemicals and establish a dose response curve between neural and non-neural cells; (3) to determine lineage-specific assay sensitivity; (4) to determine assay sensitivity (proportion of compounds that were correctly identified as neurotoxic) and specificity (proportion of compounds that were correctly identified as non-neurotoxic); and c) testing set—this set of compounds is used to identify other aspects of neurotoxicity that are not predicted by current assays and compounds with unknown neurotoxicity potential such as flame retardants and polycyclic aromatic hydrocarbons.

TABLE 4

| | | Example | |
|---|---|---|---|
| Set | Compound classes | compounds | References |
| Readout specific | inhibitory effect in | aphidicolin, | Mundy et al., Toxicology. |
| controls | proliferation | cadmium | 2010; 270:121-30 |
| | | | Culbreth et al., |
| | | | Neurotoxicology. |
| | | | 2012; 33(6): 1499-510 |
| | | | Breier et al., Toxicol Sci. |
| | | | 2008; 105(1): 119-33 |
| | stimulatory effect in | epidermal | Moors et al., Environ |
| | proliferation | growth factor | Health Perspect. 2009; |
| | | | 117(7): 1131-8 |
| | inhibitory effect in | Nocodazole, | Krug et al., Arch Toxicol. |
| | neurite outgrowth | lithium | 2013; 87(1): 123-43 |
| | | | Harrill et al., Toxicol Appl |
| | | | Pharmacol. 2013; |
| | | | 256(3): 268-80 |
| | stimulatory effect in | Y-27632 | Stiegler et al., Toxicol Sci. |
| | neurite outgrowth | | 2011; 121(1): 73-87. |
| | apoptosis induction | doxorobucin | Wang et al., Cancer Res. |
| | | | 2009; 69(2): 492-500 |
| | decrease cell viability | Staurosporine, | Chae et al., Pharmacol Res. |
| | | Camptothecin | 2000; 42(4): 373-81. |
| | | | Ulukan, Swaan. Drugs. |
| | | | 2002; 62(14): 2039-2057. |
| | Non-toxic control | Saccharin, | |
| | compounds | mannitol | |
| Training | Positive control- | Methyl mercuric | Stiegler et al., Toxicol Sci. |
| | compounds with | (II) chloride, | 2011; 121(1): 73-87 |
| | known neurotoxicity | rotenone | Krug et al., Arch Toxicol. |
| | and that have been | | 2013; 87(1): 123-43 |
| | previously identified | | |
| | by neurite outgrowth | | |
| | assay | | |

Heterogeneous populations of barcoded neural cells are imaged by mixing differentiated cells in single well to monitor expression of the different reporters by microscopy. To compare the sensitivity of hiPSC-derived neural reporter cell lineages to non-neural cells undifferentiated reporter hiPSCs are treated in parallel with the same set of compounds. Images are acquired in the presence of a readout-specific control set of compounds and these data are used to: (1) establish a image processing pipeline that includes barcode-based neural lineage identification, (2) characterize response readouts, (3) compare hiPSC versus neural lineage-specific response thresholds, and 4) differentiate general cytotoxicity from neurotoxicity.

The image processing implements a segmentation algorithm to locate cell nuclei. Applying this algorithm to each neural cell lineage (as identified by its unique barcode) enables identification and characterization of lineages of interest from a mixed population of neural cells Programs such as NenriteQuant and NeuriIQ are adapted to segment additional features such as neurites and neuronal cell shape. A single automated cell segmentation and feature extraction pipeline is used to characterize cellular parameters using various readouts. Exemplary cellular parameters and readouts (features) are shown in Table 5.

TABLE 5

| | Organelle | Cellular | |
|---|---|---|---|
| Reporter | segmentation | parameters | Examples of features |
| H2B-FP | Nuclei | Neural image | Barcoded nuclei count |
| | | Cell viability | Cell number/lineage |
| | | Proliferation | Total cell number |
| | | | Nuclei fragmentation |
| MTS-FP | Mitochondria | Apoptosis | Mitochondria area |
| palmFP | Cell | Neurite | Number of neurite processes |
| | membrane | outgrowth | Total number of processes/cell |
| | | | Mean number of processes/lineage |
| | | | Neurite outgrowth |
| | | | Length of total outgrowth/cell |
| | | | Mean of outgrowth/lineage |
| | | | Extent of branching |
| | | | Total number of branches/cell |

### Development of Dual Acceptor Cells

Genome editing tools were optimized to generate monoclonal cell lines containing two ‘acceptor sites’.

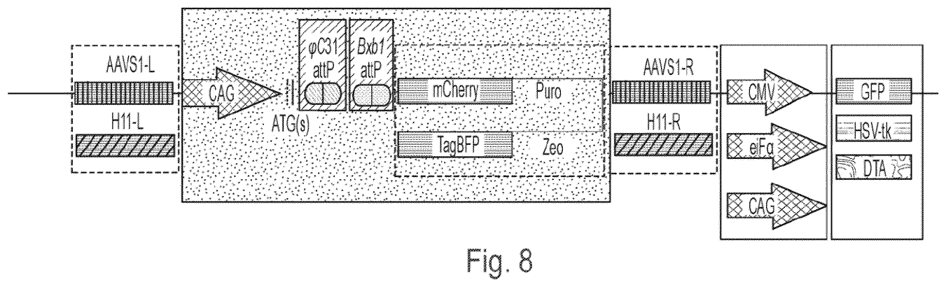

One acceptor site platform with plug and play elements contains: 1) two sequences to direct integration of the acceptor site to the genomic locus of interest, 2) a CAG constitutive promoter (that stably expresses in hiPSC), 3) 2 alternative ATGs in different frames, 4) an attP site to access recombination by PhiC31, an attP site to access recombination by BxB1, 5) an ATGless fluorescence marker fused to resistance gene, and 6) a promoter of choice and a fluorescence marker (e.g., GFP) or killer or cytotoxic gene (e.g., HSV-TK or DTA) associated to the promoter of choice and a fluorescence marker (GFP) or killer gene (HSV-TK or DTA) associated to the promoter located alter the genomic locus-right homology arm, as shown in FIG. 8.

In the dual system each acceptor site has a different genomic targeting locus, different fluorophores and selection markers (to allow for selection) and different attP sites for specific integration of the reporter platforms. In summary it allows for choice of: 1) locus of integration, 2) integrase to use for recombination of reporter construct and 3) fluorophore and selection marker and fluorophore.

Exemplary genomic loci for targeting are the AAVS1 and H11 loci. The AAVS1 locus on hiPSC for genomic integration of the acceptor site is described in example 1. The H11 locus has been shown to be an excellent locus for a wide variety of genome editing purposes. The murine Hipp11 locus was first described by Hippenmeyer et al. and further validated in mice for integrase-mediated transgenesis (Tasic et al., Proc. Natl. Acad. Sci. U.S.A. 108, 7902-7 (2011)) and human stem cells (Zhu et al., Nucleic Acids Res. 42, c34-c34 (2014)). The orthologous human H11 locus resides in an intergenic region on chromosome 22q12.2. The H11 locus does not contain any promoter thus allowing the gene of interest to be expressed under its own promoter, for example, a tissue-specific promoter to specifically express the transgene in that tissue. Transgene expression at the human H11 (hH11) locus in human embryonic stem (hES) and hiPSCs was proven to be robust and ubiquitous. The targeting efficiency at H11 is higher than typically reported frequencies and suggestive of open chromatin (Zhu et al., Nucleic Acids Rev. 42, e34-e34 (2014)). In addition, transgenes placed at the H11 locus are actively and faithfully expressed without apparent silencing for over 30 passages.

Two different integrases with mutually exclusive ail recognition sites and a high degree of specificity (Grindley et al., Annu. Rev. Biochem 75, 567-605 (2006)) are used to generate dual acceptor site cell lines, resulting in the insertion into unique genomic sites in a defined orientation and copy number. The two alternative attP sites introduced in the platform are specific for Bxb1 and PhiC31 serine recombinases. Bxb1 has been shown to have a high efficiency rate and PhiC31 has also been shown to have high recombination rates (Xu et al., BMC Biotechnol. 13, 87 (2013): Thyagarajan et al., Mol. Cell Biol. 21, 3926-3934 (2001)). Upstream of the attP sites there are the 2 ATGs in different frames that allow for transcription of the fluorescent masker fused to the resistant marker once one of them is removed. It further allows turning off the expression of the fluorophore and resistant marker once integration with the reporter takes place.

To verify successful integration of the acceptor site it also contains a GFP gene driven by CMV, PGK or CAG promoter localized downstream of the homologous-recombination region to enable prompt distinction between random and targeted integrations Cells with a randomly integrated redesigned acceptor site will fluoresce green. Cells with an integrated redesigned acceptor site—either targeted or randomly integrated—will fluoresce red or blue due to expression of mCherry or TagBP. Thus, cells with targeted integration will be exclusively red or blue, while cells with any random integration will also express GFP and be red and green or red and blue. This approach allows for the use of fluorescence microscopy to identify cells without random integration or FAC'S to sort cells without recombination, and without having to conduct a southern blot screen.

Alternatively, a negative selection marker such herpes simplex virus-thymidine kinase (HSV-TK) gene (Czako M, Marton L., Plant Physiol. 1994 March; 104(3):106-71) or diphtheria toxin A (DT-A) (Yagi T et al, Anal Biochem. 1993 October; 214(1):77-86) located outside of the homologous recombination region and is expressed by a constitutive promoter (CMV, PGK or CAG). The negative selection marker, HSV-TK in this example, will not be incorporated into the target DNA in cells that have properly undergone homologous recombination, allowing gancyclovir resistance (in the case of HSV-TK) or the diphtheria toxin expressed from the DT-A, to be used to select against recombination events that occurred through mechanisms other than HR.

Cell lines with dual acceptor sites are distinguished by expression of 2 different fluorophores (mCherry and TagBFP) and are selected using different antibiotics (puromycin and Zeocin).

Dual acceptor cell lines are engineered to have recombined two different reporters such as two neural lineage specific reporters, or two CM-lineage specific reporters. Each reporter vector has up to three fluorescently labeled transgenes which leads to the generation of dual reporter cell lines with up to six fluorescently labeled reporters Since the reporters are lineage dependent, they express the three reporters directed by the corresponding lineage promoter for each differentiated cell line.

Dual acceptor cell lines are engineered to have recombined one multicolor reporter in one locus and a Tet-On inducible Cas9 protein in the other locus. The generated cell line can be used for sgRNA library screening and validation, either individually or in pools.

The generation of hiPSC with dual acceptor sites for CM-barcode lineage allows for expression of two different lineage-specific reporters in the same cell line, thereby maximizing the number of labelled cells/lineage. Many protocols for cardiac differentiation of hiPSCs result in heterogenous pools of CMs, with varying ratios of ventricular:atrial:nodal-like cells, always consisting predominantly of ventricular-like cells (34-93%) with smaller percentage of atrial-like (2-60%) and nodal-like cells (<1-20%) (Blazeski et al., Prog. Biophys. Mol. Biol. 110, 166-77 (2012)). This is due to differences both in differentiation protocols and in how CM lineages are classified between labs. This means that for each single lineage progenitor, the number of cells expressing a barcode and therefore available for analysis is potentially quite low. With dual acceptor site hiPSC lines, ventricular with nodal and atrial with nodal reporters can be expressed in the same cell line, leading to more a homogeneous ratio of cells expressing each lineage barcode in a pooled assay. For example, the same parental cell can express ventricular cardiomyocytes with a TagBFP labelled nuclei and Nodal-like cells with an mCherry labeled nuclei. Another cell line can express atrial-like cells with a Venus labelled nuclei and nodal-like cells with an mCherry labeled nuclei. Thus, all the Nodal-like cells from both cell lines will be mCherry labeled, while the cardiomyocytes and atrial-like cells will only be labeled in cells derived from one cell line. Using this strategy there is an increase in the number of labeled nodal cells relative to other cell types, and the need to multiplex less cell lines per assay.

The generation of hiPSC with dual acceptor sites for neural-barcode lineages similarly allows expression of two different lineage-specific reporters in the same cell line, allowing improvement in the enrichment protocols used to differentiate the neural cell types into different lineages.

The improvement introduced by generating dual acceptor site cell lines also increases the flexibility for reporter design, thus allowing the insertion of larger reporter vectors.

1-167. (canceled)

168. A method of profiling two or more polypeptides in two or more cell types derived from a common cell lineage, comprising:
(i) introducing a multicistronic reporter vector from a library of multicistronic reporter vectors to an acceptor cell to generate a multireporter stem cell, wherein the acceptor cell is a pluripotent stem cell, a multipotent stem cell, or an induced pluripotent stem (iPS) cell, and wherein the multireporter reporter vector comprises:
(a) a lineage-specific promoter operably linked to an open reading frame; or
(b) a lineage-specific promoter linked to a transactivator polypeptide, and a second promoter operably linked to an open reading frame, wherein the second promoter is inducible by the transactivator polypeptide; or
(c) a lineage-specific promoter linked to a nucleic acid encoding a housekeeping polypeptide, and a constitutive promoter operably linked to an open reading frame;
wherein the open reading frame comprises two or more cistrons, and wherein expression of the open reading frame in a cell yields separate component polypeptide products from each cistron,
wherein each cistron comprises a multiple cloning site (MCS) and nucleic acid encoding a reporter vector, wherein each cistron encodes a different reporter polypeptide;
and wherein expression of two or more nucleic acids encoding polypeptides inserted into the two or more multiple cloning sites and fused to the reporter polypeptides is essentially at about 1:1: stoichiometry;
(ii) differentiating the multireporter stem cell into a plurality of differentiated multireporter cells comprising the two or more multireporter cell types following introduction of the multicistronic reporter vector to the cell; and
(iii) profiling the two or more polypeptides in the two or more multireporter cell types.

169. The method of claim 168, wherein the method further comprises determining the expression and/or location of the two or more reporter polypeptides in the two or more multireporter cell types.

170. The method of claim 168, wherein the method additionally comprises initial steps of
(i) introducing at least one recombinant nucleic acid to a cell to generate the acceptor cell, wherein the at least one recombinant nucleic acid comprises, 5′ to 3′:
(a) a first nucleic acid for targeting homologous recombination to a specific site in the cell;
(b) a first promoter;
(c) two ATG sequences;
(d) two site-specific recombinase nucleic acid;
(e) nucleic acid encoding a first reporter polypeptide and a selectable marker; and
(ii) selecting the acceptor cell, wherein only cells expressing the first reporter polypeptide but not expressing the second reporter polypeptide are selected.

171. The method of claim 170, wherein the acceptor cell is a multiacceptor cell, and the method further comprises additional initial steps of:
(iii) introducing a second recombinant nucleic acid to a cell for receiving a second multicistronic reporter vector, wherein the second recombinant nucleic acid comprises, 5′ to 3′:
(a) a third nucleic acid for targeting homologous recombination to a specific site in the cell;
(b) a third promoter;
(c) two ATG sequences;
(d) two site-specific recombinase nucleic acid;
(e) nucleic acid encoding a third reporter polypeptide and a selectable marker;
(f) fourth nucleic acid for targeting homologous recombination to a specific site in the cell;
(g) a fourth promoter; and
(h) nucleic acid encoding a fourth reporter polypeptide or cytotoxic polypeptide; and
(iv) selecting the multiacceptor cell, wherein only cells expressing the third reporter polypeptide but not the fourth reporter polypeptide are selected.

172. The method of claim 168, wherein the lineage-specific promoter is a tissue-specific promoter, and wherein the tissue-specific promoter is specific for cells of heart, blood, muscle, lung, liver, kidney, pancreas, brain, skin, or other tissue-specific lineage.

173. A method of measuring a tissue-specific toxicity response or another perturbation response to an agent in a plurality of live cells, the method comprising subjecting the plurality of cells generated from the method of claim 172 to the agent and determining the expression and/or location of the two or more reporter polypeptides in one or more cells of the plurality of cells in response to the agent.

174. The method of claim 173 wherein the tissue-specific promoter is a cardiac specific promoter, and wherein the tissue-specific toxicity response is a cardiac toxicity response.

175. The method of claim 173, wherein the tissue-specific promoter is a neural specific promoter, and wherein the tissue-specific toxicity response is a neural specific toxicity response.

176. A cell library comprising the two or more multireporter cell types generated via the method of claim 168.

177. A system, comprising:
(i) a library of multicistronic reporter vectors, wherein each multicistronic reporter vector of the library of multicistronic reporter vectors comprises:
(a) a lineage-specific promoter operably linked to an open reading frame; or
(b) a lineage-specific promoter operably linked to a transactivator polypeptide, and a second promoter operably linked to the open reading frame, wherein the second promoter is inducible by the transactivator polypeptide; or
(c) a lineage-specific promoter linked to a nucleic acid encoding a housekeeping polypeptide, and a constitutive promoter operably linked to the open reading frame;
wherein the open reading frame comprises two or more cistrons, and wherein expression of the open reading frame in a cell yields separate component polypeptide products from each cistron,
wherein each cistron comprises a multiple cloning site (MCS) and nucleic acid encoding a reporter vector, wherein each cistron encodes a different reporter polypeptide; and
wherein expression of two or more nucleic acids encoding polypeptides inserted into the two or more multiple cloning sites and fused to the reporter polypeptides is essentially at about 1:1 stoichiometry; and
(ii) a plurality of acceptor stem cells, wherein the plurality of acceptor stem cells is a plurality of pluripotent cells, a plurality of multipotent cells, or a plurality of induced pluripotent stem cells, and wherein each acceptor stem cell of the plurality of acceptor stem cells comprises a first recombinant nucleic acid for receiving a first multicistronic reporter vector integrated into a first specific site in a host cell genome.

178. The system of claim 177, wherein the plurality of acceptor cells comprise a plurality of multiacceptor cells, and wherein each multiacceptor cell of the plurality of multiacceptor cells further comprises a second recombinant nucleic acid for receiving a second expression construct, wherein the second recombinant nucleic acid is integrated into a second specific site in the host cell genome.

179. The system of claim 177, wherein the transactivator polypeptide is a tetracycline transactivator polypeptide and the second promoter comprises a tetracycline responsive element, wherein the tetracycline responsive element is a Tet operator 2 (TetO2) inducible or repressor element.

180. The system of claim 176, wherein the two or more cistrons are separated from one another by nucleic acid encoding one or more self-cleaving peptide and/or one or more internal ribosome entry site (IRES).

181. The system of claim 177, wherein at least one cistron comprises nucleic acid encoding an organelle marker.

182. The system of claim 177, wherein the one or more polypeptides comprise polypeptides that can be used to profile or distinguish a single or multiple biological pathways, cross-talk between two or more biological pathways, synthetic lethality, cellular homeostasis, organelle homeostasis, other cellular or subcellular phenotypes, cell-cell interactions or a toxicity response after differentiation of the stem cell and wherein expression of the transgene operably linked to the lineage-specific promoter is used to identify the cell type or the stage of differentiation.

183. The system of claim 182, where the profiling is performed at the single cell level.

184. The system of claim 177, wherein cells of two or more different lineages are pooled to profile the two or more polypeptides in cells of two or more different lineages, and wherein the cells of different lineages comprise lineage-specific reporter polypeptides.

185. The system of claim 178, wherein the second recombinant nucleic acid encodes a polypeptide, a reporter polypeptide, a cytotoxic polypeptide, a selectable polypeptide, a constitutive Cas9 expression vector or inducible Cas9 expression vector.

186. A method wherein a multireporter cell generated from the method of claim 167 is arrayed in a multiwell plate and used as the basis for a screen using single or oligo pool sgRNAs, a library of compounds, combination therapies, or other chemical or biological perturbations.
